(19) United States
(12) Patent Application Publication
(10) Pub. No.: US 2009/0047252 A1
(43) Pub. Date: Feb. 19, 2009

(54) Antiviral compounds

(75) Inventors: Zhenhong R. Cai, Palo Alto, CA (US); Aesop Cho, Mountain View, CA (US); Choung U. Kim, San Carlos, CA (US); Jie Xu, Foster City, CA (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/215,266

(22) Filed: Jun. 26, 2008

Related U.S. Application Data
(60) Provisional application No. 60/937,752, filed on Jun. 29, 2007.
(60) Provisional application No. 60/959,658, filed on Jul. 16, 2007.

(51) Int. Cl. A61K 31/506; C07D 403/12; C07D 401/14; C07D 413/14; C07D 403/14; A61P 31/12; A61K 31/7056; A61K 38/21; C07D 417/14; C07D 487/04; A61K 31/496; A61K 31/5377

(52) U.S. Cl. 424/85.7; 544/321; 544/317; 544/123; 544/319; 544/310; 544/212; 540/460; 514/252.19; 514/235.8; 514/272; 514/274; 514/269; 424/85.4; 514/43

(57) ABSTRACT

The invention is related to anti-viral compounds, compositions containing such compounds, and therapeutic methods that include the administration of such compounds, as well as to processes and intermediates useful for preparing such compounds.

### PRIORITY OF INVENTION

This application claims priority under 35 U.S.C. § 119(e) to U.S. Provisional Patent Application No. 60/937,752, filed 29 Jun. 2007, and to U.S. Provisional Patent Application No. 60/959,658, filed on Jul. 16, 2007. The entire content of each of these provisional patent applications is hereby incorporated herein by reference.

### FIELD OF THE INVENTION

The invention relates generally to compounds with HCV inhibitory activity.

### BACKGROUND OF THE INVENTION

Hepatitis C is recognized as a chronic viral disease of the liver which is characterized by liver disease. Although drugs targeting the liver are in wide use and have shown effectiveness, toxicity and other side effects have limited their usefulness. Inhibitors of HCV are useful to limit the establishment and progression of infection by HCV as well as in diagnostic assays for HCV.

There is a need for new HCV therapeutic agents.

### SUMMARY OF THE INVENTION

In one embodiment the invention provides a compound of the invention which is a compound of formula I:

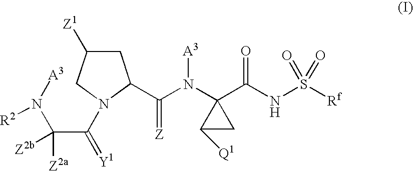

or a pharmaceutically acceptable salt, or prodrug thereof, wherein:

R¹ is independently selected from H, alkyl, alkenyl, alkynyl, aryl, cycloalkyl, heterocycle, halogen, haloalkyl, alkylsulfonamido, arylsulfonamido; —C(O)NHS(O)₂—, or —S(O)₂—, optionally substituted with one or more A³;

R² is selected from,
a) —C(Y¹)(A³),
b) (C2-10)alkyl, (C3-7)cycloalkyl or (C1-4)alkyl-(C3-7)cycloalkyl, where said cycloalkyl and alkyl-cycloalkyl may be optionally mono-, di- or tri-substituted with (C1-3)alkyl, or
   where said alkyl, cycloalkyl and alkyl-cycloalkyl may optionally be mono- or di-substituted with substituents selected from hydroxy and O—(C1-4)alkyl, or
   where each of said alkyl groups may optionally be mono-, di- or tri-substituted with halogen, or
   where each of said cycloalkyl groups being 5-, 6- or 7-membered, one or two —CH₂— groups not being directly linked to each other may be optionally replaced by —O— such that the O-atom is linked to the N atom to which R² is attached via at least two C-atoms,
c) phenyl, (C1-3)alkyl-phenyl, heteroaryl or (C1-3)alkyl-heteroaryl,
   wherein the heteroaryl groups are 5- or 6-membered having from 1 to 3 heteroatoms selected from N, O and S, wherein said phenyl and heteroaryl groups may optionally be mono-, di- or trisubstituted with substituents selected from halogen, —OH, (C1-4)alkyl, O—(C1-4)alkyl, S—(C1-4)alkyl, —NH₂, —CF₃, —NH((C1-4)alkyl) and —N((C1-4)alkyl)₂, —CONH₂ and —CONH—(C1-4)alkyl; and wherein said (C1-3)alkyl may optionally be substituted with one or more halogen;
d) —S(O)₂(A³); or
e) —C(Y¹)—X—Y;

each R³ is independently H or (C1-6)alkyl;

Y¹ is independently O, S, N(A³), N(O)(A³), N(OA³), N(O)(OA³) or N(N(A³)(A³));

Z is O, S, or NR³;

each R_{c} is R⁴, H, cyano, F, Cl, Br, I, —C(═O)NR_{d}R_{c}, C(═O)NRₛRₜ, NR^{s}Rₜ, S(═O)₂NRₛRₜ, (C1-10)alkoxy, cycloalkyl, aryl, or heteroaryl, which aryl or heteroaryl is optionally substituted with one or more groups independently selected from halo, hydroxy, (C1-10)alkyl, (C2-10)alkenyl, (C2-10)alkynyl, (C1-10)alkanoyl, (C1-10)alkoxy, (C1-10)alkanoyloxy, (C1-10)alkoxycarbonyl, NRₙRₚ, SRᵣ, S(O)Rᵣ, or S(O)₂Rᵣ;

R_{d} and Rₑ are each independently H or (C1-10)alkyl;

Z^{2b} is H, (C1-10)alkyl, (C2-10)alkenyl, or (C2-10)alkynyl;

Q¹ is (C1-10)alkyl, (C2-10)alkenyl, or (C2-10)alkynyl which Q¹ is optionally substituted with R⁴ or R_{c}; or Q¹ and Z^{2a} taken together with the atoms to which they are attached form a heterocycle, which heterocycle may optionally be substituted with one or more oxo (═O), R⁴, or A³;

each X is independently a bond, O, S, or NR³;

Y is a polycarbocycle or a polyheterocycle, which polycarbocycle or a polyheterocycle is optionally substituted with one or more R⁴, halo, carboxy, hydroxy, (C1-10)alkyl, (C2-10)alkenyl, (C2-10)alkynyl, (C1-10)alkanoyl, (C1-10)alkoxy, (C1-10)alkanoyloxy, (C1-10)alkoxycarbonyl, NRₙRₚ, SRᵣ, S(O)Rᵣ, or S(O)₂Rᵣ;

each R⁴ is independently —P(Y³)(OA²)(OA²), —P(Y³)(OA²)(N(A²)₂), —P(Y³)(A²)(OA²), —P(Y³)(A²)(N(A²)₂), or P(Y³)(N(A²)₂)(N(A²)₂);

each Y³ is independently O, S, or NR³;

each Rₙ and Rₚ is independently H, (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, which (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, is optionally substituted with one or more R¹, halo, hydroxy, carboxy, cyano, or (C1-10)alkoxy; or Rₙ and Rₚ together with the nitrogen to which they are attached form a pyrrolidine, piperidine, piperazine, morpholino, or thiomorpholino ring;

each Rᵣ is independently H, (C1-10)alkyl, (C2-10)alkenyl, (C2-10)alkynyl, (C1-10)alkanoyl, heterocycle, or (C1-10)alkoxycarbonyl, wherein any (C1-10)alkyl, (C2-10)alkenyl, (C2-10)alkynyl, (C1-10)alkanoyl, heterocycle, or (C1-10)alkoxycarbonyl is optionally substituted with one or more A³;

each Rₛ and Rₜ is independently H, (C1-10)alkyl, (C2-10)alkenyl, (C2-10)alkynyl, (C1-10)alkanoyl, S(═O)₂A², (C1-10)alkoxy, (C1-10)alkanoyloxy, or (C1-10)alkoxycarbonyl, which (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, is optionally substituted with one or more R¹, halo hydroxy, carboxy, cyano, or (C1-10)alkoxy; or Rₛ and Rₜ together with the nitrogen to which they are attached form a pyrrolidine, piperidine, piperazine, morpholino, or thiomorpholino ring wherein one or more carbon atoms of said pyrrolidine, piperidine, piperazine, morpholino or thiomorpholino ring is optionally replaced by S(═O), S(═O)₂, or C(═O);

Z^{2a} is H, (C1-10)alkyl, (C2-10)alkenyl, (C2-10)alkynyl, haloalkyl, (C1-10)alkyl-S(═O)₂—(C1-10)alkyl, or cycloalkyl, wherein any carbon atom of Z^{2a} may optionally be replaced with a heteroatom selected from O, S or N and wherein any cycloalkyl is optionally substituted with one or more (C1-10)alkyl, (C2-10)alkenyl, (C2-10)alkynyl, F, Cl, Br, or I; or Z^{2a} optionally forms a heterocycle with one or more R¹, R², Q¹, or A³;

each A³ is independently selected from PRT, H, —OH, —C(O)OH, cyano, alkyl, alkenyl, alkynyl, amino, amido, imido, imino, halogen, CF₃, —OCF₃, CH₂CF₃, cycloalkyl, nitro, aryl, aralkyl, alkoxy, aryloxy, heterocycle, —C(A²)₃, —C(A²)₂-C(O)A², —C(O)A², —C(O)OA², —O(A²), —N(A²)₂, —S(A²), —CH₂P(Y¹)(A²)(OA²), —CH₂P(Y¹)(A²)(N(A²)₂), —CH₂P(Y¹)(OA²)(OA²), —OCH₂P(Y¹)(OA²)(OA²), —OCH₂P(Y¹)(A²)(OA²), —OCH₂P(Y¹)(A²)(N(A²)₂), —C(O)OCH₂P(Y¹)(OA²)(OA²), —C(O)OCH₂P(Y¹)(A²)(OA²), —C(O)OCH₂P(Y¹)(A²)(N(A²)₂), —CH₂P(Y¹)(OA²)(N(A²)₂), —OCH₂P(Y¹)(OA²)(N(A²)₂), —C(O)OCH₂P(Y¹)(OA²)(N(A²)₂), —CH₂P(Y¹)(N(A²)₂)(N(A²)₂), —C(O)OCH₂P(Y¹)(N(A²)₂)(N(A²)₂), —OCH₂P(Y¹)(N(A²)₂)(N(A²)₂), —(CH₂)ₘ-heterocycle, —(CH₂)ₘC(O)Oalkyl, —O—(CH₂)ₙ—O—C(O)—Oalkyl, —O—(CH₂)ᵣ—O—C(O)—(CH₂)ₘ-alkyl, —(CH₂)ₘO—C(O)—O-alkyl, —(CH₂)ₘO—C(O)—O-cycloalkyl, —N(H)C(Me)C(O)O-alkyl, SRᵣ, S(O)Rᵣ, S(O)₂Rᵣ, Si(R³), or alkoxy arylsulfonamide,

wherein each A³ maybe optionally substituted with 1 to 4
—R¹, —P(Y¹)(OA²)(OA²), —P(Y¹)(OA²)(N(A²)₂), —P(Y¹)(A²)(OA²), —P(Y¹)(A²)(N(A²)₂), or P(Y¹)(N(A²)₂)(N(A²)₂), —C(═O)N(A²)₂), halogen, alkyl, alkenyl, alkynyl, aryl, carbocycle, heterocycle, aralkyl, arylsulfonamide, aryl alkylsulfonamide, aryloxy sulfonamide, aryloxyalkylsulfonamide, aryloxy arylsulfonamide, alkyl sulfonamide, alkyloxysulfonamide, alkyloxy alkylsulfonamide, arylthio, —(CH₂)ₘheterocycle, (CH₂)ₘ—C(O)O-alkyl, —O(CH₂)ₘOC(O)Oalkyl, —O—(CH₂)ₘ—O—C(O)(CH₂)ₘ-alkyl, —(CH₂)ₘ—O—C(O)—O-alkyl, —(CH₂)ₘ—O—C(O)—O-cycloalkyl, —N(H)C(CH₃)C(O)O-alkyl, or alkoxy arylsulfonamide, optionally substituted with R¹;

optionally each independent instance of A³ and Q¹ can be taken together with one or more A³ or Q¹ groups to form a ring;

A² is independently selected from PRT, H, alkyl, alkenyl, alkynyl, amino, amino acid, alkoxy, aryloxy, cyano, haloalkyl, cycloalkyl, aryl, heteroaryl, heterocycle, alkylsulfonamide, or arylsulfonamide, wherein each A² is optionally substituted with A³;

R^{f} is H, alkyl, alkenyl, alkynyl, aryl, heteroaryl, or cycloalkyl, which R^{f} is optionally substituted with one or more R_{g};

each R_{g} is independently alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;

each Rₕ and Rᵢ is independently H, alkyl, or haloalkyl;

m is 0 to 6;

Z¹ is -L¹-A⁴-L²-A⁵;

L¹ is a bond, (C1-10)alkyl, O, S, —C(═O)—, —C(═O)O—, —OC(═O)—, —C(═O)NR³—, —NR³C(═O)—, —S(O)—, —S(O)₂—, —NR S(O)₂—, —S(O)₂NR³—, or NR³;

A⁴ is a monocyclic heteroaryl containing 1, 2, or 3 N, which A⁴ is optionally substituted with one or more A³;

L² is (C1-10)alkyl, O, S, —C(═O)—, —C(═O)O—, —OC(═O)—, —C(═O)NR³—, —NR³C(═O)—, —S(O)—, —S(O)₂—, —NR³S(O)₂—, —S(O)₂NR³—, or NR³; and

A⁵ is aryl, alkyl, cycloalkyl, or heteroaryl, which A⁵ is optionally substituted with one or more A³.

The present invention also provides a pharmaceutical composition comprising a compound of the invention and at least one pharmaceutically acceptable carrier.

The present invention also provides a pharmaceutical composition for use in treating disorders associated with HCV.

The present invention also provides a pharmaceutical composition further comprising a nucleoside analog.

The present invention also provides for a pharmaceutical composition further comprising an interferon or pegylated interferon.

The present invention also provides for a pharmaceutical composition wherein said nucleoside analogue is selected from ribavirin, viramidine levovirin, a L-nucleoside, and isatoribine and said interferon is a-interferon or pegylated interferon.

The present invention also provides for a method of treating disorders associated with hepatitis C, said method comprising administering to an individual a pharmaceutical composition which comprises a therapeutically effective amount of a compound of the invention.

The present invention also provides a method of inhibiting HCV, comprising administering to a mammal afflicted with a condition associated with HCV activity, an amount of a compound of the invention, effective to inhibit HCV.

The present invention also provides a compound of the invention for use in medical therapy (preferably for use in inhibiting HCV or treating a condition associated with HCV activity), as well as the use of a compound of the invention for the manufacture of a medicament useful for inhibiting HCV or the treatment of a condition associated with HCV activity in a mammal.

The present invention also provides synthetic processes and novel intermediates disclosed herein which are useful for preparing compounds of the invention. Some of the compounds of the invention are useful to prepare other compounds of the invention.

In another aspect the invention provides a method of inhibiting HCV activity in a sample comprising treating the sample with a compound of the invention.

In one embodiment the invention provides a compound having improved inhibitory or pharmacokinetic properties, including enhanced activity against development of viral resistance, improved oral bioavailability, greater potency or extended effective half-life in vivo. Certain compounds of the invention may have fewer side effects, less complicated dosing schedules, or be orally active.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the embodiments.

### Compounds of the Invention

The compounds of the invention exclude compounds heretofore known. However it is within the invention to use compounds that previously were not known to have antiviral properties for antiviral purposes (e.g. to produce an anti-viral effect in an animal). With respect to the United States, the compounds or compositions herein exclude compounds that are anticipated under 35 USC §102 or that are obvious under 35 USC §103.

Whenever a compound described herein is substituted with more than one of the same designated group, e.g., “R¹” or “A³”, then it will be understood that the groups may be the same or different, i.e., each group is independently selected.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and cyclopropylmethyl

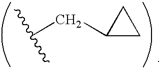

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to, ethylene or vinyl (—CH═CH₂), allyl (—CH₂CH═CH₂), cyclopentenyl (—C₅H₇), and 5-hexenyl (—CH₂ CH₂CH₂CH₂CH═CH₂).

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to, acetylenic (—C≡CH) and propargyl (—CH₂C≡CH),

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to, methylene (—CH₂—) 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to, 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to, acetylene (—C≡C—), propargyl (—CH₂C≡C—), and 4-pentynyl (—CH₂CH₂CH₂C≡CH—).

“Aryl” means a monovalent aromatic hydrocarbon radical of 6-20 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, naphthylmethyl, 2-naphthylethan-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

The term “polycarbocycle” refers to a saturated or unsaturated polycyclic ring system having from about 6 to about 25 carbon atoms and having two or more rings (e.g. 2, 3, 4, or 5 rings). The rings can be fused and/or bridged to form the polycyclic ring system. For example, the term includes bicyclo [4,5], [5,5], [5,6] or [6,6] ring systems, as well as the following bridged ring systems:

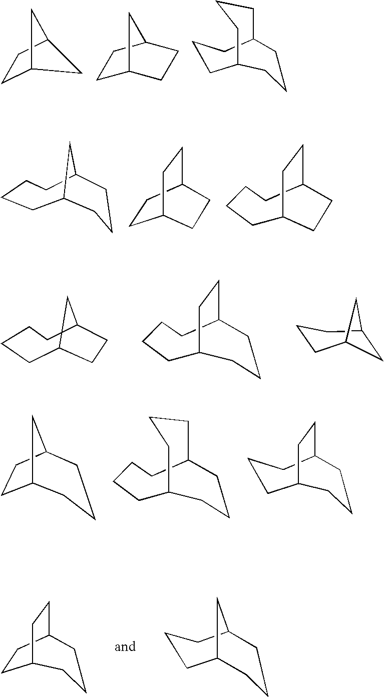

(i.e., [2.1.1], [2.2.1], [3.3.3], [4.3.1], [2.2.2], [4.2.2], [4.2.1], [4.3.2], [3.1.1], [3.2.1], [4.3.3], [3.3.2], [3.2.2] and [3.3.1] polycyclic rings, respectively) that can be linked to the remainder of the compound of formula (I) through any synthetically feasible position. Like the other polycarbocycles, these representative bicyclo and fused ring systems can optionally comprise one or more double bonds in the ring system.

The term “polyheterocycle” refers to a polycarbocycle as defined herein, wherein one or more carbon atoms is replaced with a heteroatom (e,g, O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂R^{x}, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo.

“Substituted alkyl”, “substituted aryl”, and “substituted arylalkyl” mean alkyl, aryl, and arylalkyl respectively, in which one or more hydrogen atoms are each independently replaced with a non-hydrogen substituent. Typical substituents include, but are not limited to, —X, —R, —O⁻, —OR, —SR, —S—, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR—S(═O)₂O⁻, —S(═O)₂OH, —S(═O)₂R, —OS(═O)₂OR, —S(═O)₂NR, —S(═O)R, —OP(═O)O₂RR, —P(═O)O₂RR—P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen: F, Cl, Br, or I; and each R is independently —H, alkyl, aryl, heterocycle, protecting group or prodrug moiety. Alkylene, alkenylene, and alkynylene groups may also be similarly substituted.

The term “optionally substituted” in reference to a particular moiety of the compound of formula I, (e.g., an optionally substituted aryl group) refers to a moiety having 0, 1, 2, or more substituents.

“Heterocycle” as used herein includes by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. In one specific embodiment of the invention “heterocycle” includes a “carbocycle” as defined herein, wherein one or more (e.g. 1, 2, 3, or 4) carbon atoms have been replaced with a heteroatom (e.g. O, N, or S). The term heterocycle includes heteroaryl rings.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, imidazole, triazole, bis-tetrahydrofuranyl

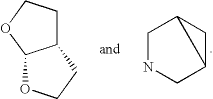

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Heteroaryl” means a monovalent aromatic radical of one or more carbon atoms and one or more atoms selected from the group consisting of N, O, S and P, derived by the removal of one hydrogen atom from a single atom of a parent aromatic ring system. Heteroaryl groups include monocycles having 3 to 7 ring members (2 to 6 carbon atoms and 1 to 3 heteroatoms selected from the group consisting of N, O, P and S) and bicyclic rings having 7 to 10 ring members (4 to 9 carbon atoms and 1 to 3 heteroatoms selected from the group consisting of N, O, P and S). Heteroaryl bicyclic rings typically have 7 to 10 ring atoms (6 to 9 carbon atoms and 1 to 2 heteroatoms selected from the group consisting of N, O and S) arranged as a bicyclo [4,5], [5,5], [5,6], or [6,6] system; or 9 to 10 ring atoms (8 to 9 carbon atoms and 1 to 2 hetero atoms selected from the group consisting of N and S) arranged as a bicyclo [5,6] or [6,6] system. The heteroaryl group may be bonded through a carbon, nitrogen, sulfur, phosphorus or other atom by a stable covalent bond. Heteroaryl groups include, for example: pyridyl, pyridazinyl, pyrimidinyl, pyrazinyl, s-triazinyl, oxazolyl, imidazolyl, thiazolyl, isoxazolyl, pyrazolyl, isothiazolyl, furanyl, thiofuranyl, thienyl, and pyrrolyl rings.

“Carbocycle” refers to a saturated, unsaturated or aromatic ring having up to about 25 carbon atoms. Typically, a carbocycle has about 3 to 7 carbon atoms as a monocycle, about 7 to 12 carbon atoms as a bicycle, and up to about 25 carbon atoms as a polycycle. Monocyclic carbocycles typically have 3 to 6 ring atoms, still more typically 5 or 6 ring atoms. Bicyclic carbocycles typically have 7 to 12 ring atoms, e.g., arranged as a bicyclo [4,5], [5,5], [5,6] or [6,6] system, or 9 or 10 ring atoms arranged as a bicyclo [5,6] or [6,6] system. The term carbocycle includes “cycloalkyl” which is a saturated or unsaturated carbocycle. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl. When Q¹ and Z^{2a} taken together with the atoms to which they are attached form a heterocycle, the heterocycle formed by Q¹ and Z^{2a} taken together with the atoms to which they are attached may typically comprise up to about 25 atoms.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “treatment” or “treating,” to the extent it relates to a disease or condition includes preventing the disease or condition from occurring, inhibiting the disease or condition, eliminating the disease or condition, and/or relieving one or more symptoms of the disease or condition.

The term “PRT” is selected from the terms “prodrug moiety” and “protecting group” as defined herein.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity. The invention includes all stereoisomers of the compounds described herein.

### Prodrugs

The term “prodrug” as used herein refers to any compound that when administered to a biological system generates the drug substance, i.e. active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound.

“Prodrug moiety” refers to a labile functional group which separates from the active inhibitory compound during metabolism, systemically, inside a cell, by hydrolysis, enzymatic cleavage, or by some other process (Bundgaard, Hans, “Design and Application of Prodrugs” in A Textbook of Drug Design and Development (1991), P. Krogsgaard-Larsen and H. Bundgaard, Eds. Harwood Academic Publishers, pp. 113-191). Enzymes which are capable of an enzymatic activation mechanism with the phosphonate prodrug compounds of the invention include, but are not limited to, amidases, esterases, microbial enzymes, phospholipases, cholinesterases, and phosphases. Prodrug moieties can serve to enhance solubility, absorption and lipophilicity to optimize drug delivery, bioavailability and efficacy. A prodrug moiety may include an active metabolite or drug itself.

Exemplary prodrug moieties include the hydrolytically sensitive or labile acyloxymethyl esters —CH₂C(═O)R⁹ and acyloxymethyl carbonates —CH₂C(═O)OR⁹ where R⁹ is C₁-C₆ alkyl, C₁-C₆ substituted alkyl, C₆-C₂₀ aryl or C₆-C₂₀ substituted aryl. The acyloxyalkyl ester was first used as a prodrug strategy for carboxylic acids and then applied to phosphates and phosphonates by Farquhar et al. (1983) J. Pharm. Sci. 72: 324; also U.S. Pat. Nos. 4,816,570, 4,968,788, 5,663,159 and 5,792,756. Subsequently, the acyloxyalkyl ester was used to deliver phosphonic acids across cell membranes and to enhance oral bioavailability. A close variant of the acyloxyalkyl ester, the alkoxycarbonyloxyalkyl ester (carbonate), may also enhance oral bioavailability as a prodrug moiety in the compounds of the combinations of the invention. An exemplary acyloxymethyl ester is pivaloyloxymethoxy, (POM) —CH₂C(═O)C(CH₃)₃. An exemplary acyloxymethyl carbonate prodrug moiety is pivaloyloxymethylcarbonate (POC) —CH₂C(═O)OC(CH₃)₃.

Aryl esters of phosphorus groups, especially phenyl esters, are reported to enhance oral bioavailability (De Lombaert et al. (1994) J. Med. Chem. 37: 498). Phenyl esters containing a carboxylic ester ortho to a phosphate have also been described (Khamnei and Torrence, (1996) J. Med. Chem. 39:4109-4115). Benzyl esters are reported to generate parent phosphonic acids. In some cases, substituents at the ortho- or para-position may accelerate the hydrolysis. Benzyl analogs with an acylated phenol or an alkylated phenol may generate the phenolic compound through the action of enzymes, e.g., esterases, oxidases, etc., which in turn undergoes cleavage at the benzylic C—O bond to generate phosphoric acid and a quinone methide intermediate. Examples of this class of prodrugs are described by Mitchell et al. (1992) J. Chem. Soc. Perkin Trans. 112345; Glazier WO 91/19721. Still other benzylic prodrugs have been described containing a carboxylic ester-containing group attached to the benzylic methylene (Glazier WO 91/19721). Thio-containing prodrugs are reported to be useful for the intracellular delivery of phosphonate drugs. These proesters contain an ethylthio group in which the thiol group is either esterified with an acyl group or combined with another thiol group to form a disulfide. Deesterification or reduction of the disulfide generates the free thio intermediate which subsequently breaks down to the phosphoric acid and episulfide (Puech et al. (1993) Antiviral Res., 22: 155-174; Benzaria et al. (1996) J. Med. Chem. 39: 4958).

### Protecting Groups

In the context of the present invention, protecting groups include prodrug moieties and chemical protecting groups.

“Protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

Protecting groups are available, commonly known and used, and are optionally used to prevent side reactions with the protected group during synthetic procedures, i.e. routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. The PG groups do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene (John Wiley & Sons, Inc., New York, 1991, ISBN 0-471-62301-6) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below.

By way of example and not limitation, A³, A² and R¹ are all recursive substituents in certain embodiments. Typically, each of these may independently occur 20, 19, 18, 17, 16, 15, 14, 13, 12, 11, 10, 9, 8, 7, 6, 5, 4, 3, 2, 1, or 0, times in a given embodiment. More typically, each of these may independently occur 12 or fewer times in a given embodiment. Whenever a compound described herein is substituted with more than one of the same designated group, e.g., “R¹” or “A³”, then it will be understood that the groups may be the same or different, i.e., each group is independently selected. Wavy lines indicate the site of covalent bond attachments to the adjoining groups, moieties, or atoms.

In one embodiment of the invention, the compound is in an isolated and purified form. Generally, the term “isolated and purified” means that the compound is substantially free from biological materials (e.g. blood, tissue, cells, etc.). In one specific embodiment of the invention, the term means that the compound or conjugate of the invention is at least about 50 wt. % free from biological materials; in another specific embodiment, the term means that the compound or conjugate of the invention is at least about 75 wt. % free from biological materials; in another specific embodiment, the term means that the compound or conjugate of the invention is at least about 90 wt. % free from biological materials; in another specific embodiment, the term means that the compound or conjugate of the invention is at least about 98 wt. % free from biological materials; and in another embodiment, the term means that the compound or conjugate of the invention is at least about 99 wt. % free from biological materials. In another specific embodiment, the invention provides a compound or conjugate of the invention that has been synthetically prepared (e.g., ex vivo).

### Cellular Accumulation

In one embodiment, the invention provides compounds capable of accumulating in human hepatocytes. The compounds of this embodiment may further comprise a phosphonate or phosphonate prodrug. More typically, the phosphonate or phosphonate prodrug can have the structure A³ as described herein.

### Stereoisomers

The compounds of the invention may have chiral centers, e.g., chiral carbon or phosphorus atoms. The compounds of the invention thus include racemic mixtures of all stereoisomers, including enantiomers, diastereomers, and atropisomers. In addition, the compounds of the invention include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the invention. The racemic mixtures are separated into their individual, substantially optically pure isomers through well-known techniques such as, for example, the separation of diastereomeric salts formed with optically active adjuncts, e.g., acids or bases followed by conversion back to the optically active substances. In most instances, the desired optical isomer is synthesized by means of stereospecific reactions, beginning with the appropriate stereoisomer of the desired starting material.

The compounds of the invention can also exist as tautomeric isomers in certain cases. Although only one delocalized resonance structure may be depicted, all such forms are contemplated within the scope of the invention. For example, ene-amine tautomers can exist for purine, pyrimidine, imidazole, guanidine, amidine, and tetrazole systems and all their possible tautomeric forms are within the scope of the invention.

### Salts and Hydrates

Examples of physiologically acceptable salts of the compounds of the invention include salts derived from an appropriate base, such as an alkali metal (for example, sodium), an alkaline earth metal (for example, magnesium), ammonium and NX₄⁺ (wherein X is C₁-C₄ alkyl). Physiologically acceptable salts of a hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of a hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds of the invention will typically be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present invention.

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, typically amines, or to acidic groups. Finally, it is to be understood that the compositions herein comprise compounds of the invention in their un-ionized, as well as zwitterionic form, and combinations with stoichiometric amounts of water as in hydrates.

Also included within the scope of this invention are the salts of the parental compounds with one or more amino acids. Any of the natural or unnatural amino acids are suitable, especially the naturally-occurring amino acids found as protein components, although the amino acid typically is one bearing a side chain with a basic or acidic group, e.g., lysine, arginine or glutamic acid, or a neutral group such as glycine, serine, threonine, alanine, isoleucine, or leucine.

### Methods of Inhibition of HCV

Another aspect of the invention relates to methods of inhibiting the activity of HCV comprising the step of treating a sample suspected of containing HCV with a compound or composition of the invention.

Compounds of the invention may act as inhibitors of HCV, as intermediates for such inhibitors or have other utilities as described below. The inhibitors will generally bind to locations on the surface or in a cavity of the liver. Compounds binding in the liver may bind with varying degrees of reversibility. Those compounds binding substantially irreversibly are ideal candidates for use in this method of the invention. Once labeled, the substantially irreversibly binding compounds are useful as probes for the detection of HCV. Accordingly, the invention relates to methods of detecting NS3 in a sample suspected of containing HCV comprising the steps of: treating a sample suspected of containing HCV with a composition comprising a compound of the invention bound to a label; and observing the effect of the sample on the activity of the label. Suitable labels are well known in the diagnostics field and include stable free radicals, fluorophores, radioisotopes, enzymes, chemiluminescent groups and chromogens. The compounds herein are labeled in conventional fashion using functional groups such as hydroxyl or amino. In one embodiment the invention provides a compound of formula (I) that comprises or that is bound or linked to one or more detectable labels. Within the context of the invention samples suspected of containing HCV include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically the sample will be suspected of containing HCV. Samples can be contained in any medium including water and organic solvent/water mixtures. Samples include living organisms such as humans, and man made materials such as cell cultures.

The treating step of the invention comprises adding the compound of the invention to the sample or it comprises adding a precursor of the composition to the sample. The addition step comprises any method of administration as described above.

If desired, the activity of HCV after application of the compound can be observed by any method including direct and indirect methods of detecting HCV activity. Quantitative, qualitative, and semiquantitative methods of determining HCV activity are all contemplated. Typically one of the screening methods described above are applied, however, any other methods such as observation of the physiological properties of a living organism are also applicable.

Many organisms contain HCV. The compounds of this invention are useful in the treatment or prophylaxis of conditions associated with HCV activation in animals or in man.

However, in screening compounds capable of inhibiting HCV it should be kept in mind that the results of enzyme assays may not always correlate with cell culture assays. Thus, a cell based assay should typically be the primary screening tool.

### Screens for HCV Inhibitors

Compounds of the invention are screened for inhibitory activity against HCV by any of the conventional techniques for evaluating enzyme activity. Within the context of the invention, typically compounds are first screened for inhibition of HCV in vitro and compounds showing inhibitory activity are then screened for activity in vivo. Compounds having in vitro Ki (inhibitory constants) of less then about 5×10⁻⁶ M, typically less than about 1×10⁻⁷ M and preferably less than about 5×10⁻⁸ M are preferred for in vivo use. Useful in vitro screens have been described in detail.

### Pharmaceutical Formulations

The compounds of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of conditions associated with HCV activity.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### Routes of Administration

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

### Combination Therapy

Active ingredients of the invention can also be used in combination with other active ingredients. Such combinations are selected based on the condition to be treated, cross-reactivities of ingredients and pharmaco-properties of the combination.

It is also possible to combine any compound of the invention with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together.

Suitable active therapeutic agents or ingredients which can be combined with the compounds of formula I can include interferons, e.g., pegylated rIFN-alpha 2b, pegylated rIFN-alpha 2a, rIFN-alpha 2b, IFN alpha-2b XL, rIFN-alpha 2a, consensus IFN alpha, infergen, rebif, locteron, AVI-005, PEG-infergen, pegylated IFN-beta, oral interferon alpha, feron, reaferon, intermax alpha, rIFN-beta, infergen+actimmune, IFN-omega with DUROS, and albuferon; ribavirin analogs, e.g., rebetol, copegus, levovirin VX-497, and viramidine (taribavirin); NS5a inhibitors, e.g., A-831 and A-689; NS5b polymerase inhibitors, e.g., NM-283, valopicitabine, R1626, PSI-6130 (R1656), HCV-796, BILB 1941, MK-0608, NM-107, R7128, VCH-759, PF-868554, GSK625433, and XTL-2125; NS3 protease inhibitors, e.g., SCH-503034 (SCH-7), VX-950 (Telaprevir), ITMN-191, and BILN-2065; alpha-glucosidase 1 inhibitors, e.g., MX-3253 (celgosivir) and UT-231B; hepatoprotectants, e.g., IDN-6556, ME 3738, MitoQ, and LB-84451; non-nucleoside inhibitors of HCV, e.g., benzimidazole derivatives, benzo-1,2,4-thiadiazine derivatives, and phenylalanine derivatives; and other drugs for treating HCV, e.g., zadaxin, nitazoxanide (alinea), BIVN-401 (virostat), DEBIO-025, VGX-410C, EMZ-702, AVI 4065, bavituximab, oglufanide, PYN-17, KPE02003002, actilon (CPG-10101), KRN-7000, civacir, GI-5005, ANA-975 (isatoribine), XTL-6865, ANA 971, NOV-205, tarvacin, EHC-18, and NIM811.

In yet another embodiment, the present application discloses pharmaceutical compositions comprising a compound of the present invention, or a pharmaceutically acceptable salt, solvate, and/or ester thereof, in combination with at least one additional therapeutic agent, and a pharmaceutically acceptable carrier or excipient.

According to the present invention, the therapeutic agent used in combination with the compound of the present invention can be any agent having a therapeutic effect when used in combination with the compound of the present invention. For example, the therapeutic agent used in combination with the compound of the present invention can be interferons, ribavirin analogs, NS3 protease inhibitors, NS5b polymerase inhibitors, alpha-glucosidase 1 inhibitors, hepatoprotectants, non-nucleoside inhibitors of HCV, and other drugs for treating HCV.

In another embodiment, the present application provides pharmaceutical compositions comprising a compound of the present invention, or a pharmaceutically acceptable salt, solvate, and/or ester thereof, in combination with at least one additional therapeutic agent selected from the group consisting of pegylated rIFN-alpha 2b, pegylated rIFN-alpha 2a, rIFN-alpha 2b, IFN alpha-2b XL, rIFN-alpha 2a, consensus IFN alpha, infergen, rebif, locteron, AVI-005, PEG-infergen, pegylated IFN-beta, oral interferon alpha, feron, reaferon, intermax alpha, r-IFN-beta, infergen+actimmune, IFN-omega with DUROS, albuferon, rebetol, copegus, levovirin, VX-497, viramidine (taribavirin), A-831, A-689, NM-283, valopicitabine, R1626, PSI-6130 (R1656), HCV-796, BILB 1941, MK-0608, NM-107, R7128, VCH-759, PF-868554, GSK625433, XTL-2125, SCH-503034 (SCH-7), VX-950 (Telaprevir), ITMN-191, and BILN-2065, MX-3253 (celgosivir), UT-231B, IDN-6556, ME 3738, MitoQ, and LB-84451, benzimidazole derivatives, benzo-1,2,4-thiadiazine derivatives, and phenylalanine derivatives, zadaxin, nitazoxanide (alinea), BIVN-401 (virostat), DEBIO-025, VGX-410C, EMZ-702, AVI 4065, bavituximab, oglufanide, PYN-17, KPE02003002, actilon (CPG-10101), KRN-7000, civacir, GI-5005, ANA-975 (isatoribine), XTL-6865, ANA 971, NOV-205, tarvacin, EHC-18, and NIM811 and a pharmaceutically acceptable carrier or excipient.

In yet another embodiment, the present application provides a combination pharmaceutical agent comprising:

a) a first pharmaceutical composition comprising a compound of the present invention, or a pharmaceutically acceptable salt, solvate, or ester thereof; and

b) a second pharmaceutical composition comprising at least one additional therapeutic agent selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, interferons, ribavirin analogs, NS3 protease inhibitors, alpha-glucosidase 1 inhibitors, hepatoprotectants, non-nucleoside inhibitors of HCV, and other drugs for treating HCV, and combinations thereof.

Combinations of the compounds of formula I and additional active therapeutic agents may be selected to treat patients infected with HCV and other conditions such as HIV infections. Accordingly, the compounds of formula I may be combined with one or more compounds useful in treating HIV, for example HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, interferons, ribavirin analogs, NS3 protease inhibitors, NS5b polymerase inhibitors, alpha-glucosidase 1 inhibitors, hepatoprotectants, non-nucleoside inhibitors of HCV, and other drugs for treating HCV.

More specifically, one or more compounds of the present invention may be combined with one or more compounds selected from the group consisting of 1) HIV protease inhibitors, e.g., amprenavir, atazanavir, fosamprenavir, indinavir, lopinavir, ritonavir, lopinavir+ritonavir, nelfinavir, saquinavir, tipranavir, brecanavir, darunavir, TMC-126, TMC-114, mozenavir (DMP-450), JE-2147 (AG1776), AG1859, DG35, L-756423, R00334649, KNI-272, DPC-681, DPC-684, and GW640385X, DG17, PPL-100, 2) a HIV non-nucleoside inhibitor of reverse transcriptase, e.g., capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirine), efavirenz, BILR 355 BS, VRX 840773, UK-453,061, RDEA806, 3) a HIV nucleoside inhibitor of reverse transcriptase, e.g., zidovudine, emtricitabine, didanosine, stavudine, zalcitabine, lamivudine, abacavir, amdoxovir, elvucitabine, alovudine, MIV-210, racivir (±-FTC), D-d4FC, emtricitabine, phosphazide, fozivudine tidoxil, fosalvudine tidoxil, apricitibine (AVX754), amdoxovir, KP-1461, abacavir+lamivudine, abacavir+lamivudine+zidovudine, zidovudine+lamivudine, 4) a HIV nucleotide inhibitor of reverse transcriptase, e.g., tenofovir, tenofovir disoproxil fumarate+emtricitabine, tenofovir disoproxil fumarate+emtricitabine+efavirenz, and adefovir, 5) a HIV integrase inhibitor, e.g., curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-707035, MK-2048, BA-011, BMS-538158, GSK364735C, 6) a gp41 inhibitor, e.g., enfuvirtide, sifuvirtide, FB006M, TRI-1144, SPC3, DES6, Locus gp41, CovX, and REP 9, 7) a CXCR4 inhibitor, e.g., AMD-070, 8) an entry inhibitor, e.g., SP01A, TNX-355, 9) a gp120 inhibitor, e.g., BMS-488043 and BlockAide/CR, 10) a G6PD and NADH-oxidase inhibitor, e.g., immunitin, 10) a CCR5 inhibitor, e.g., aplaviroc, vicriviroc, INCB9471, PRO-140, INCB15050, PF-232798, CCR5 mAb004, and maraviroc, 11) an interferon, e.g., pegylated rIFN-alpha 2b, pegylated rIFN-alpha 2a, rIFN-alpha 2b, IFN alpha-2b XL, rIFN-alpha 2a, consensus IFN alpha, infergen, rebif, locteron, AVI-005, PEG-infergen, pegylated IFN-beta, oral interferon alpha, feron, reaferon, intermax alpha, r-IFN-beta, infergen+actimmune, IFN-omega with DUROS, and albuferon, 12) ribavirin analogs, e.g., rebetol, copegus, levovirin, VX-497, and viramidine (taribavirin) 13) NS5a inhibitors, e.g., A-831 and A-689, 14) NS5b polymerase inhibitors, e.g., NM-283, valopicitabine, R1626, PSI-6130 (R1656), HCV-796, BILB 1941, MK-0608, NM-107, R7128, VCH-759, PF-868554, GSK625433, and XTL-2125, 15) NS3 protease inhibitors, e.g., SCH-503034 (SCH-7), VX-950 (Telaprevir), ITMN-191, and BILN-2065, 16) alpha-glucosidase 1 inhibitors, e.g., MX-3253 (celgosivir) and UT-231B, 17) hepatoprotectants, e.g., IDN-6556, ME 3738, MitoQ, and LB-84451, 18) non-nucleoside inhibitors of HCV, e.g., benzimidazole derivatives, benzo-1,2,4-thiadiazine derivatives, and phenylalanine derivatives, 19) other drugs for treating HCV, e.g., zadaxin, nitazoxanide (alinea), BIVN-401 (virostat), DEBIO-025, VGX-410C, EMZ-702, AVI 4065, bavituximab, oglufanide, PYN-17, KPE02003002, actilon (CPG-10101), KRN-7000, civacir, GI-5005, ANA-975 (isatoribine), XTL-6865, ANA 971, NOV-205, tarvacin, EHC-18, and NIM811, 19) pharmacokinetic enhancers, e.g., BAS-100 and SPI452, 20)RNAse H inhibitors, e.g., ODN-93 and ODN-112, 21) other anti-HIV agents, e.g., VGV-1, PA-457 (bevirimat), ampligen, HRG214, cytolin, polymun, VGX-410, KD247, AMZ 0026, CYT 99007, A-221 HIV, BAY 50-4798, MDXO10 (iplimumab), PBS119, ALG889, and PA-1050040.

### Metabolites of the Compounds of the Invention

Also falling within the scope of this invention are the in vivo metabolic products of the compounds described herein. Such products may result for example from the oxidation, reduction, hydrolysis, amidation, esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radiolabelled (e.g., C¹⁴ or H³) compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the compounds of the invention even if they possess no HCV-inhibitory activity of their own.

Methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The phosphonate prodrugs of the invention typically will be stable in the digestive system but are substantially hydrolyzed to the parental drug in the digestive lumen, liver or other metabolic organ, or within cells in general.

### Exemplary Methods of Making the Compounds of the Invention.

The invention also relates to methods of making the compositions of the invention. The compositions are prepared by any of the applicable techniques of organic synthesis. Many such techniques are well known in the art. However, many of the known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, jr., 1980; Vol. 5, Leroy G. Wade, Jr., 1984; and Vol. 6, Michael B. Smith; as well as March, J., Advanced Organic Chemistry, Third Edition, (John Wiley & Sons, New York, 1985), Comprehensive Organic Synthesis. Selectivity Strategy & Efficiency in Modern Organic Chemistry. In 9 Volumes, Barry M. Trost, Editor-in-Chief (Pergamon Press, New York, 1993 printing). Other methods suitable for preparing compounds of the invention are described in International Patent Application Publication Number WO 2006/020276.

A number of exemplary methods for the preparation of the compositions of the invention are provided below. These methods are intended to illustrate the nature of such preparations and are not intended to limit the scope of applicable methods.

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically the temperatures will be −100° C. to 200° C., solvents will be aprotic or protic, and reaction times will be 10 seconds to 10 days. Work-up typically consists of quenching any unreacted reagents followed by partition between a water/organic layer system (extraction) and separating the layer containing the product.

Oxidation and reduction reactions are typically carried out at temperatures near room temperature (about 20° C.), although for metal hydride reductions frequently the temperature is reduced to 0° C. to −100° C., solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions).

Standard synthetic techniques such as azeotropic removal of reaction by-products and use of anhydrous reaction conditions (e.g., inert gas environments) are common in the art and will be applied when applicable.

The terms “treated”, “treating”, “treatment”, and the like, when used in connection with a chemical synthetic operation, mean contacting, mixing, reacting, allowing to react, bringing into contact, and other terms common in the art for indicating that one or more chemical entities is treated in such a manner as to convert it to one or more other chemical entities. This means that “treating compound one with compound two” is synonymous with “allowing compound one to react with compound two”, “contacting compound one with compound two”, “reacting compound one with compound two”, and other expressions common in the art of organic synthesis for reasonably indicating that compound one was “treated”, “reacted”, “allowed to react”, etc., with compound two. For example, treating indicates the reasonable and usual manner in which organic chemicals are allowed to react. Normal concentrations (0.01M to 10M, typically 0.1M to 1M), temperatures (−100° C. to 250° C., typically −78° C. to 150° C., more typically −78° C. to 100° C., still more typically 0° C. to 100° C.), reaction vessels (typically glass, plastic, metal), solvents, pressures, atmospheres (typically air for oxygen and water insensitive reactions or nitrogen or argon for oxygen or water sensitive), etc., are intended unless otherwise indicated. The knowledge of similar reactions known in the art of organic synthesis are used in selecting the conditions and apparatus for “treating” in a given process. In particular, one of ordinary skill in the art of organic synthesis selects conditions and apparatus reasonably expected to successfully carry out the chemical reactions of the described processes based on the knowledge in the art.

Modifications of each of the exemplary schemes and in the examples (hereafter “exemplary schemes”) leads to various analogs of the specific exemplary materials produce. The above-cited citations describing suitable methods of organic synthesis are applicable to such modifications.

In each of the exemplary schemes it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium, and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point, and molecular weight in distillation and sublimation, presence or absence of polar functional groups in chromatography, stability of materials in acidic and basic media in multiphase extraction, and the like. One skilled in the art will apply techniques most likely to achieve the desired separation.

A single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller, C. H., (1975) J. Chromatogr., 113, 3) 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers of atropisomeric compounds can be separated and isolated by normal- and reverse-phase chromatography following methods for separation of atropisomeric naphthyl-isoquinolines (Hoye, T., WO 96/15111). By method (3), a racemic mixture of two enantiomers can be separated by chromatography using a chiral stationary phase (Chiral Liquid Chromatography (1989) W. J. Lough, Ed. Chapman and Hall, New York; Okamoto, (1990) J. of Chromatogr. 513:375-378). Enriched or purified enantiomers can be distinguished by methods used to distinguish other chiral molecules with asymmetric carbon atoms, such as optical rotation and circular dichroism.

### SPECIFIC EMBODIMENTS OF THE INVENTION

In one specific embodiment of the invention R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

In one specific embodiment of the invention R^{f} is cyclopropyl.

In one specific embodiment of the invention R^{f} is 1-methylcyclopropyl.

In one specific embodiment the invention provides a compound of formula (II):

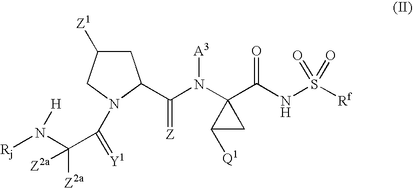

or a pharmaceutically acceptable salt, or prodrug thereof, wherein: Rⱼ is tert-butoxycarbonyl, cyclopentyloxycarbonyl, 2,2,2-trifluoro-1,1-dimethylethyl, 1-methylcyclopropyloxycarbonyl, 2-(N,N-dimethylamino)-1-1-dimethylethoxycarbonyl, 2-morpholino-1-1-dimethylethoxycarbonyl, tetrahydrofur-3-yloxycarbonyl, or

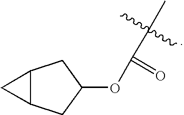

In one specific embodiment of the invention Z is O; Y¹ is O; and one of Z^{2a} and Z^{2b} is hydrogen.

In one specific embodiment of the invention Q¹ is vinyl, ethyl, cyanomethyl, propyl, 2-fluoroethyl, 2,2-difluoroethyl, or 2-cyanoethyl.

In one specific embodiment of the invention Q¹ and Z^{2a} taken together with the atoms to which they are attached form a 12-18 membered heterocycle, which heterocycle may optionally be substituted with one or more oxo (═O) or A³.

In one specific embodiment the invention provides a compound of formula (III):

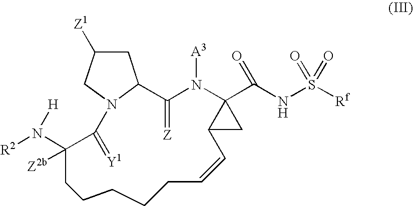

or a pharmaceutically acceptable salt, or prodrug thereof.

In one specific embodiment the invention provides a compound of formula (IV):

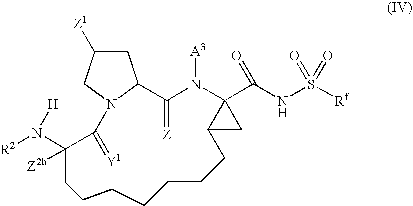

or a pharmaceutically acceptable salt, or prodrug thereof.

In one specific embodiment of the invention Z^{2a} is tert-butyl, 1-methylcyclohexyl, tetrahydropyran-4-yl, 1-methylcyclohexyl, 4,4-difluorocyclohexyl, 2,2,2-trifluoro-1-trifluoromethylethyl, or cyclopropyl.

In one specific embodiment of the invention X is O, S, or NR³.

In one specific embodiment of the invention X is O.

In one specific embodiment of the invention L¹ is O.

In one specific embodiment of the invention L² is O, S, or NR³.

In one specific embodiment of the invention L² is O.

In one specific embodiment of the invention L² is S.

In one specific embodiment of the invention L² is NH.

In one specific embodiment of the invention A⁴ is selected from:

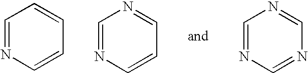

wherein A⁴ is optionally substituted with one or more A³ and each of L¹ and L² is independently connected to a carbon atom of A⁴.

In one specific embodiment of the invention A⁵ is aryl which A⁵ is optionally substituted with one or more A³.

In one specific embodiment of the invention A⁵ is heteroaryl which A⁵ is optionally substituted with one or more A³.

In one specific embodiment of the invention A⁵ is cycloalkyl which A⁵ is optionally substituted with one or more A³.

In one specific embodiment of the invention -L²-A⁵ is selected from:

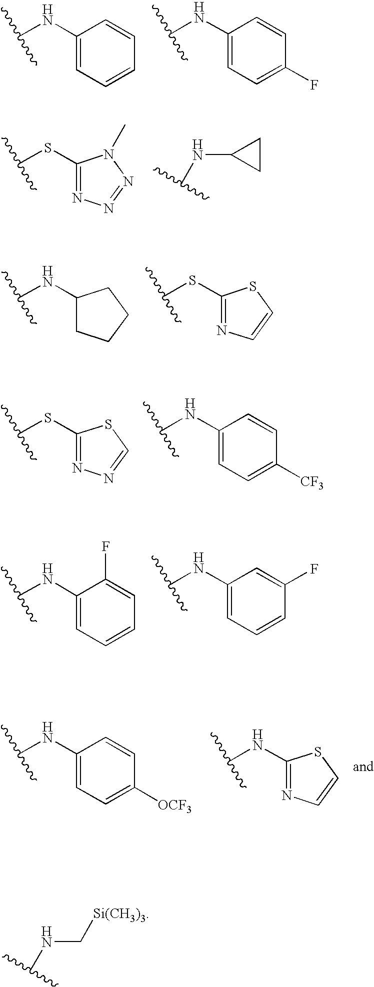

In one specific embodiment of the invention Z¹ is selected from:

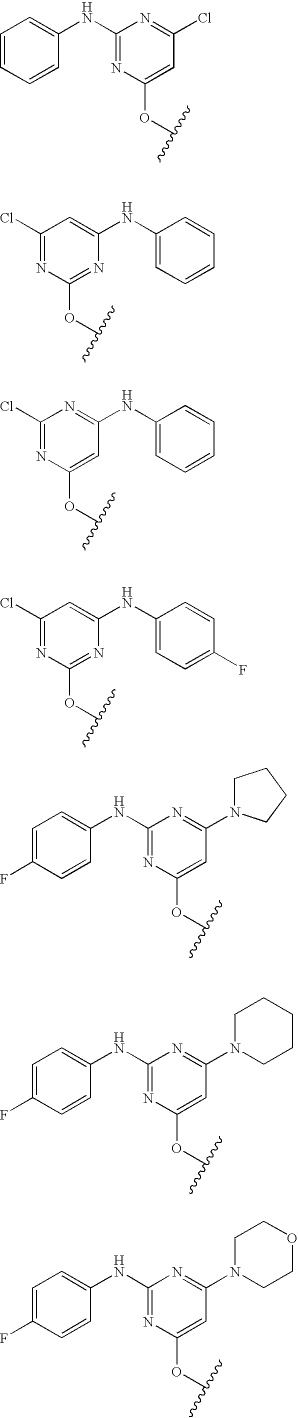

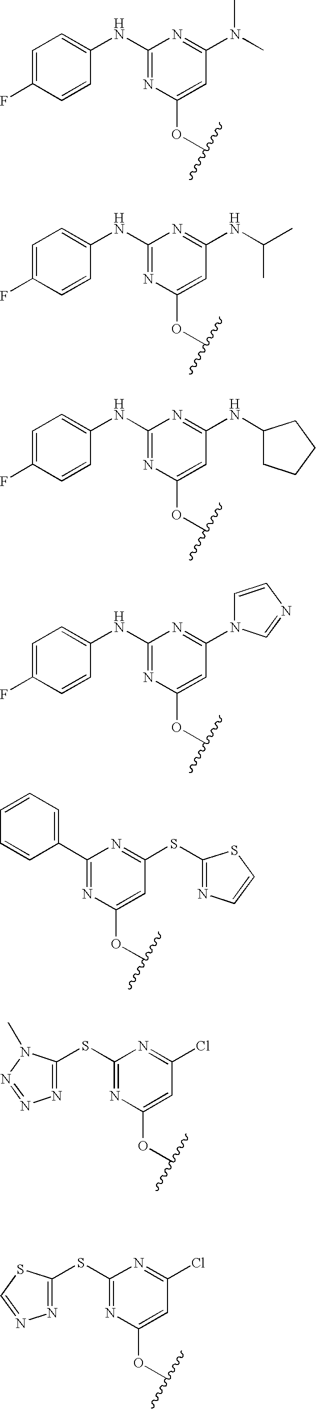

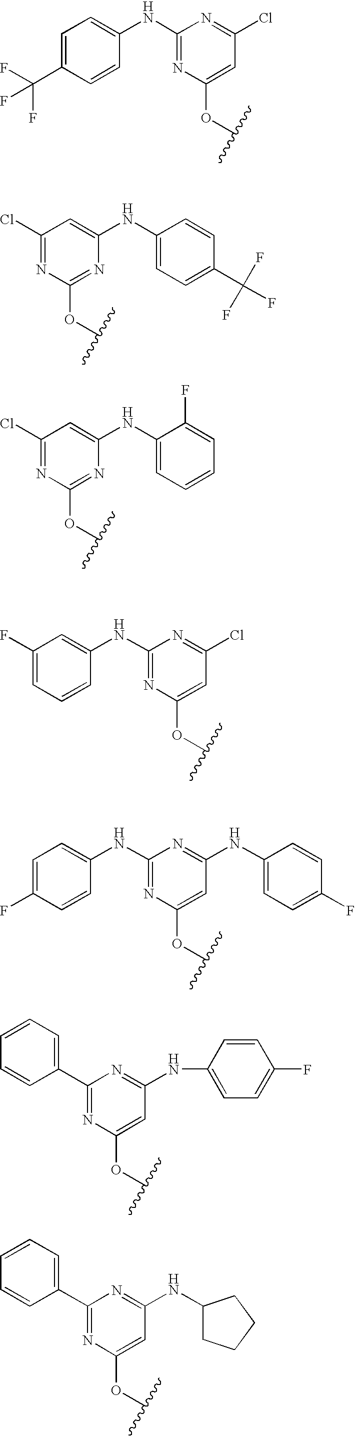

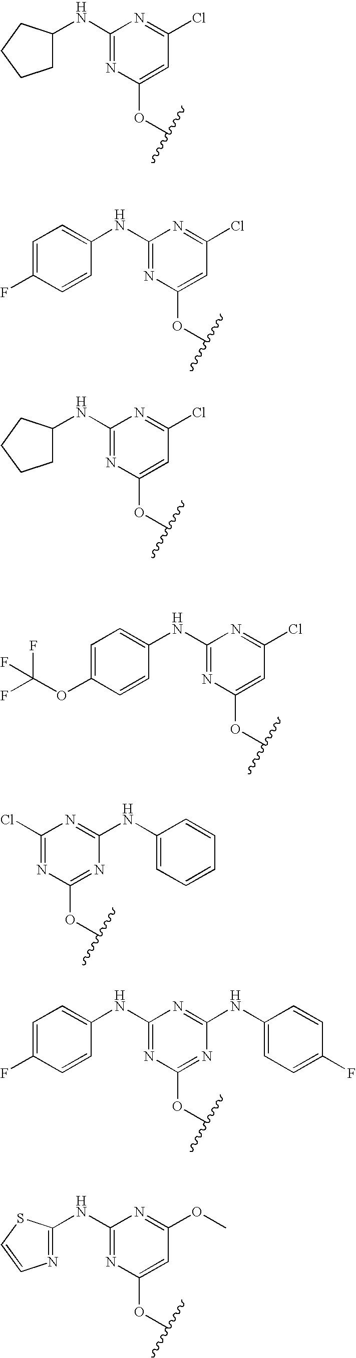

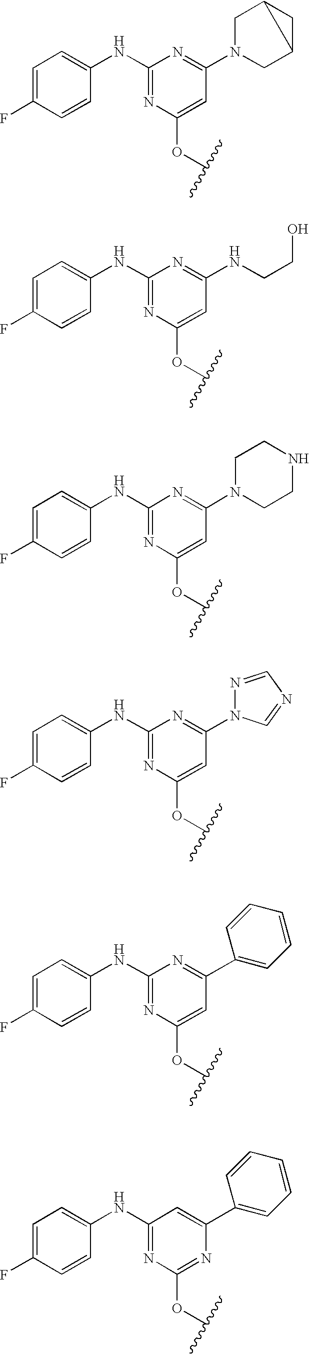

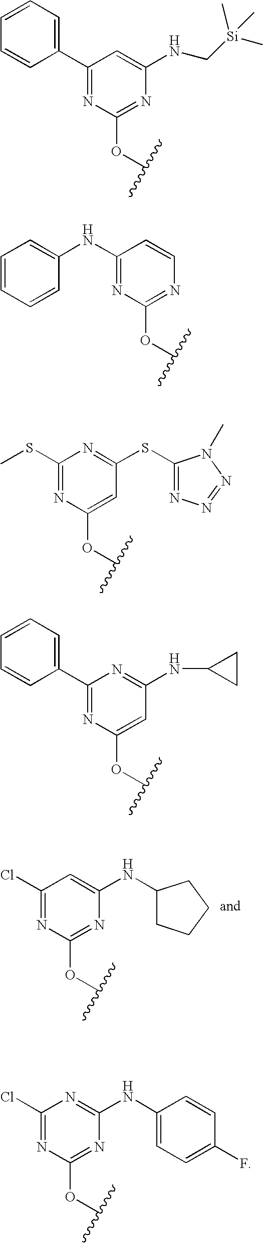

In one specific embodiment of the invention A⁵ is aryl, (C2-C10)alkyl, cycloalkyl, or heteroaryl, which A⁵ is optionally substituted with one or more A³.

In one specific embodiment of the invention A⁵ is aryl, cycloalkyl, or heteroaryl, which A⁵ is optionally substituted with one or more A³.

In one specific embodiment of the invention A⁵ is alkyl or cycloalkyl substituted with one or more A³.

In one specific embodiment of the invention A⁵ is alkyl or cycloalkyl substituted with Si(R³).

In one specific embodiment of the invention Y is a polycarbocycle.

In one specific embodiment of the invention Y is polyheterocycle.

In one specific embodiment of the invention Y is a fused carbocyclic ring system.

In one specific embodiment of the invention Y is a fused heterocyclic ring system.

In one specific embodiment of the invention Y is a fused carbocyclic ring system comprising one or more double bonds.

In one specific embodiment of the invention Y is a fused heterocyclic ring system comprising one or more double bonds.

In one specific embodiment of the invention Y is a bridged carbocyclic ring system.

In one specific embodiment of the invention Y is a bridged heterocyclic ring system.

In one specific embodiment of the invention Y is a bridged carbocyclic ring system comprising one or more double bonds.

In one specific embodiment of the invention Y is a bridged heterocyclic ring system comprising one or more double bonds.

In one specific embodiment of the invention Y comprises a bridged ring system selected from:

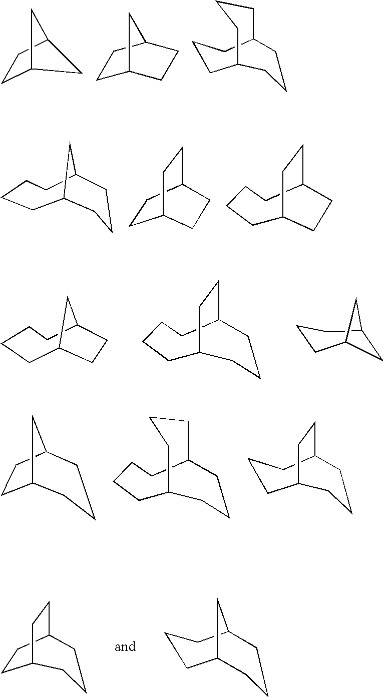

wherein one or more carbon atoms in the bridged ring system is optionally replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo; and wherein the ring system optionally comprises one or more double bonds. In one specific embodiment of the invention the ring system comprises one or more double bonds. In one specific embodiment of the invention one or more carbon atoms in the bridged ring system is replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂R^{x}, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo.

In one specific embodiment of the invention Y comprises a fused ring system selected from:

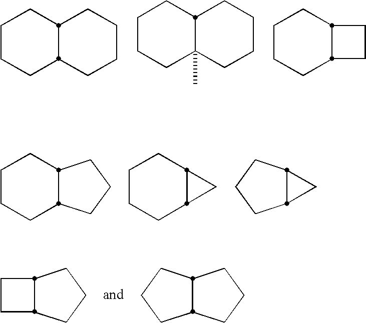

wherein one or more carbon atoms in the fused ring system is optionally replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo; and wherein the ring system optionally comprises one or more double bonds. In one specific embodiment of the invention one or more carbon atoms in the bridged ring system is replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo.

In one specific embodiment of the invention Y is selected from:

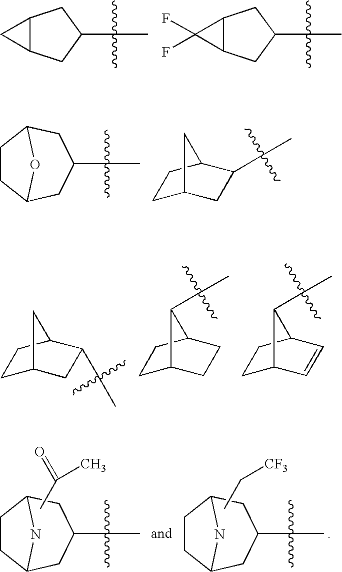

In one embodiment the heteroaryl of R¹ is a 5- or 6-membered ring having from 1 to 3 heteroatoms selected from N, O and S.

In another embodiment the heteroaryl of A⁴ is a monocyclic heteroaryl containing 1, 2, or 3 N.

In a specific embodiment the invention provides a compound which is

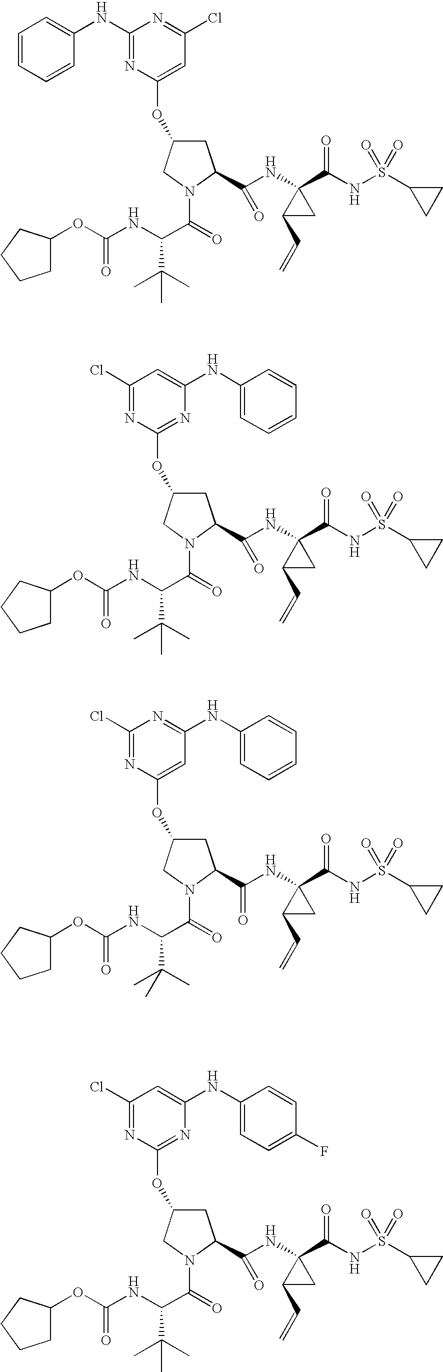

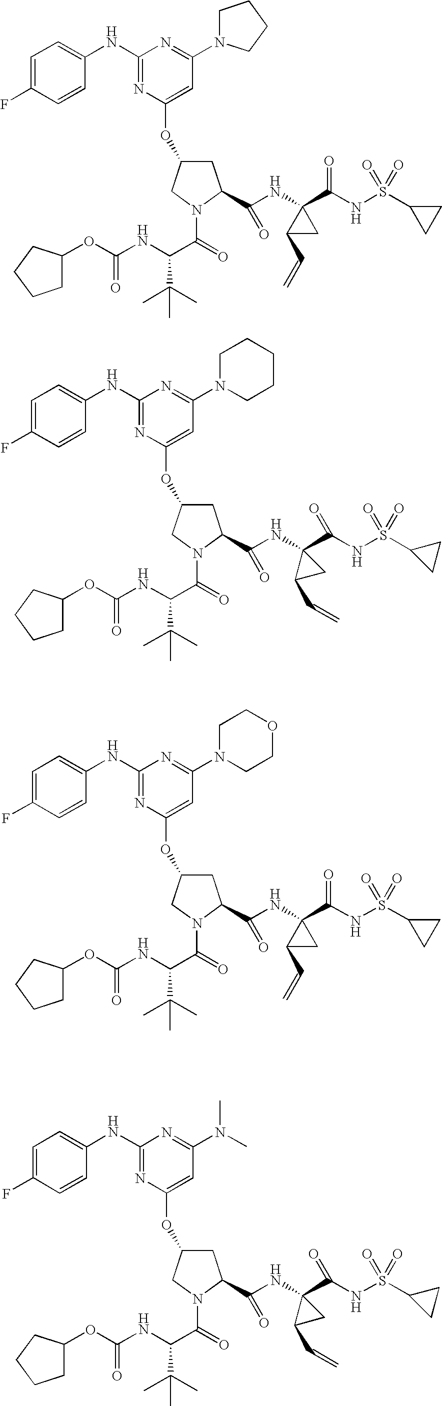

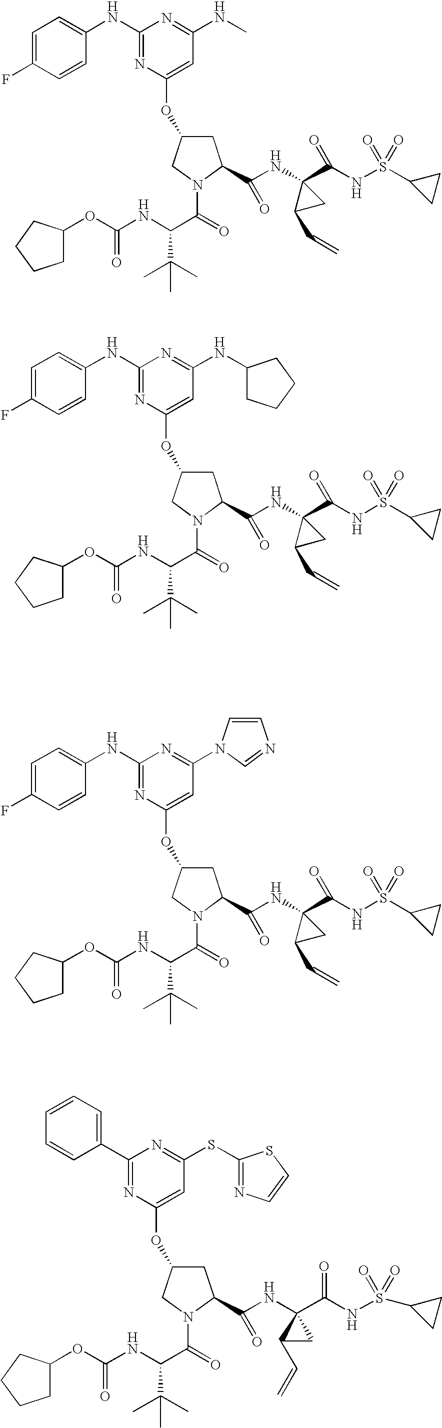

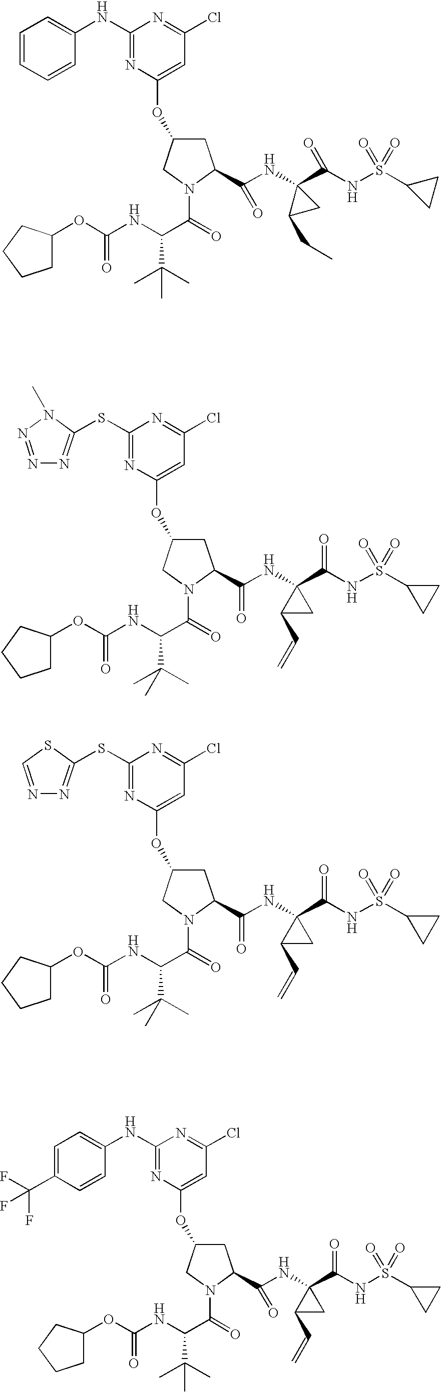

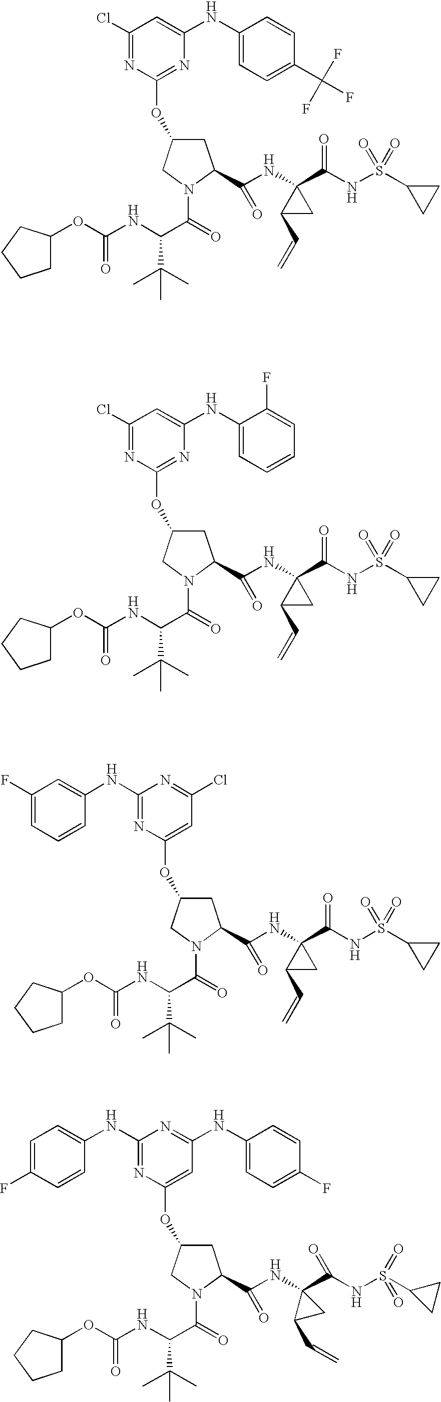

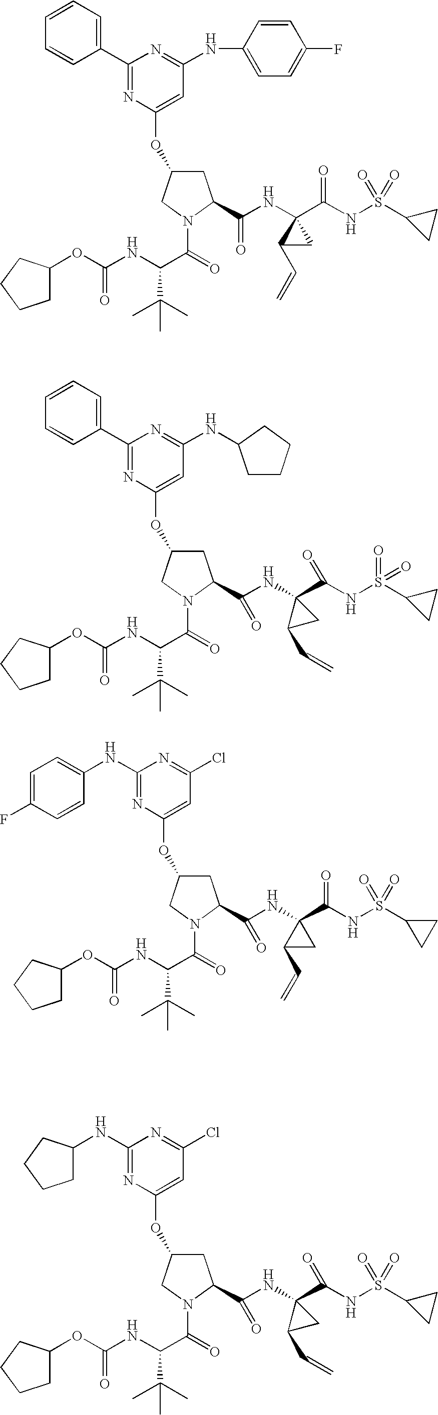

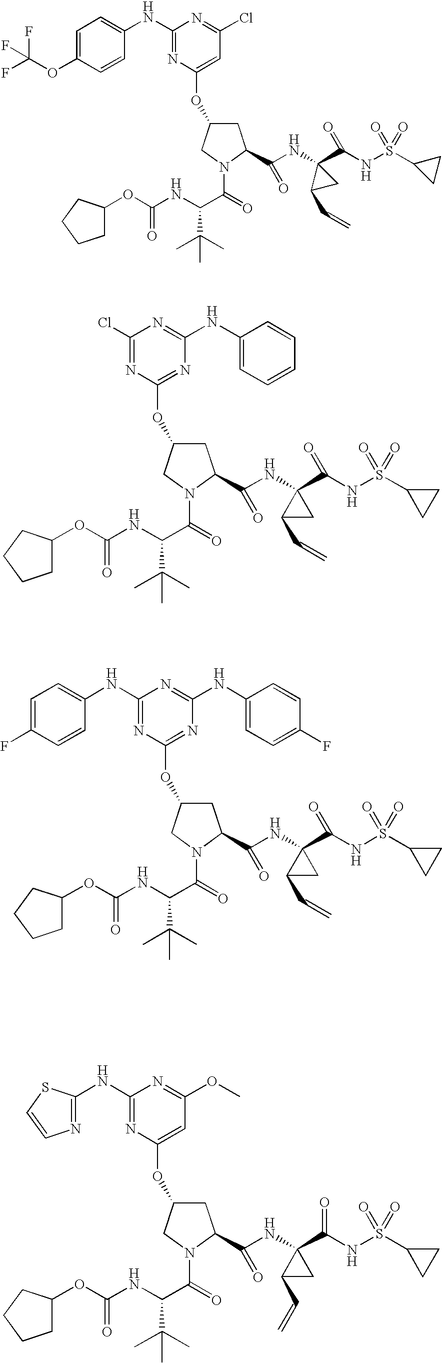

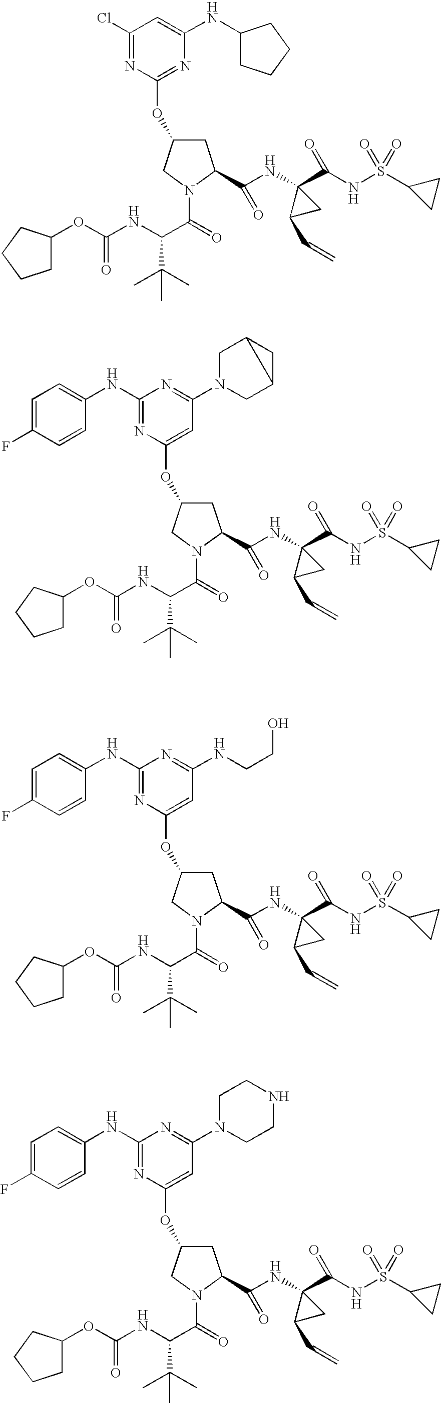

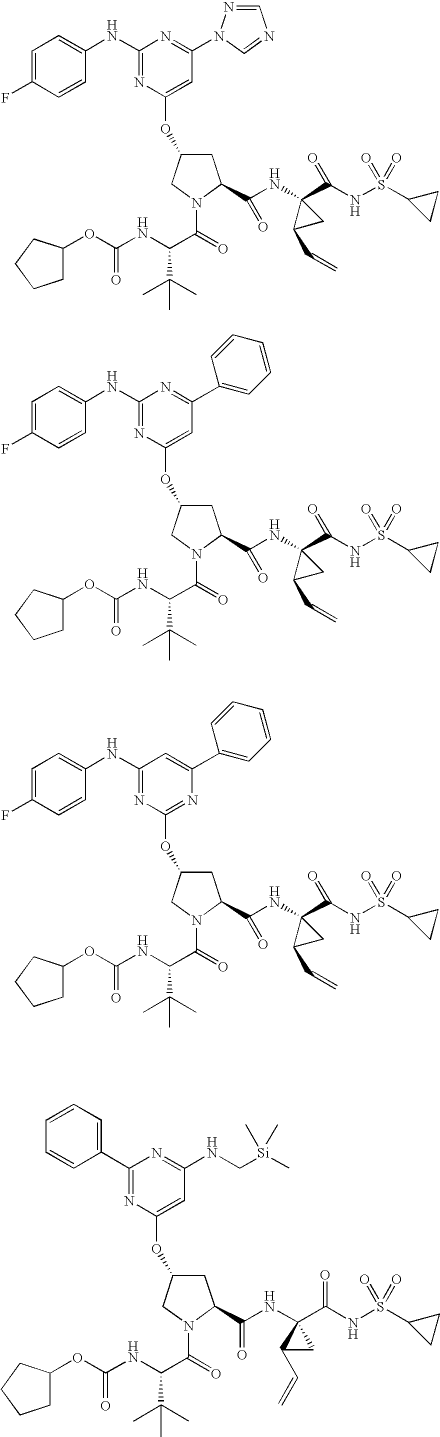

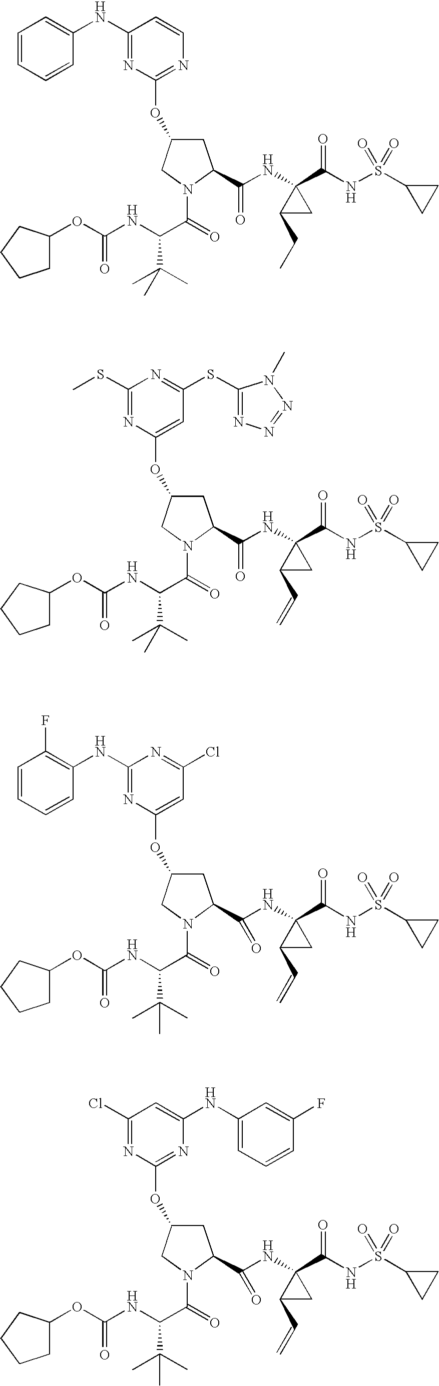

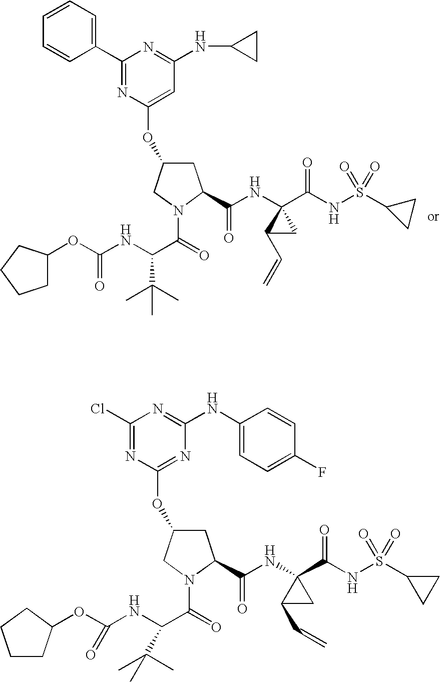

or a pharmaceutically acceptable salt, or prodrug thereof.

In a specific embodiment the invention provides a compound which is

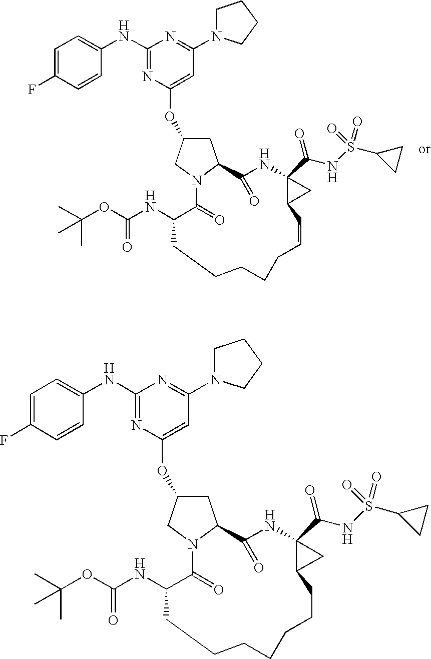

or a pharmaceutically acceptable salt, or prodrug thereof.

### Specific Embodiment 1

In one specific embodiment the invention provides a compound of formula I:

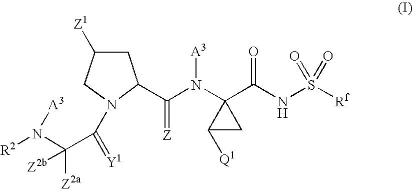

or a pharmaceutically acceptable salt, or prodrug thereof, wherein:

R¹ is independently selected from H, alkyl, alkenyl, alkynyl, aryl, cycloalkyl, heterocycle, halogen, haloalkyl, alkylsulfonamido, arylsulfonamido, —C(O)NHS(O)₂—, or —S(O)₂—, optionally substituted with one or more A³;

R² is selected from,

a) —C(Y¹)(A³),

b) (C2-10)alkyl, (C3-7)cycloalkyl or (C1-4)alkyl-(C3-7)cycloalkyl, where said cycloalkyl and alkyl-cycloalkyl may be optionally mono-, di- or tri-substituted with (C1-3)alkyl, or where said alkyl, cycloalkyl and alkyl-cycloalkyl may optionally be mono- or di-substituted with substituents selected from hydroxy and O—(C1-4)alkyl, or

where each of said alkyl groups may optionally be mono-, di- or tri-substituted with halogen, or where each of said cycloalkyl groups being 5-, 6- or 7-membered, one or two —CH₂— groups not being directly linked to each other may be optionally replaced by —O— such that the O-atom is linked to the N atom to which R² is attached via at least two C-atoms,

c) phenyl, (C1-3)alkyl-phenyl, heteroaryl or (C1-3)alkyl-heteroaryl,

wherein the heteroaryl groups are 5- or 6-membered having from 1 to 3 heteroatoms selected from N, O and S, wherein said phenyl and heteroaryl groups may optionally be mono-, di- or trisubstituted with substituents selected from halogen, —OH, (C1-4)alkyl, O—(C1-4)alkyl, S—(C1-4)alkyl, —NH₂, —CF₃, —NH((C1-4)alkyl) and —N((C1-4)alkyl)₂, —CONH₂ and —CONH—(C1-4)alkyl; and wherein said (C1-3)alkyl may optionally be substituted with one or more halogen;

d) —S(O)₂(A³); or

e) —C(Y¹)—X—Y;

each R³ is independently H or (C1-6)alkyl;

Y¹ is independently O, S, N(A³), N(O)(A³), N(OA³), N(O)(OA³) or N(N(A³)(A³));

Z is O, S, or NR³;

each R_{c} is R⁴, H, cyano, F, Cl, Br, I, —C(═O)NR_{d}Rₑ, C(═O)NRₛRₜ, NRₛRₜ, S(═O)₂NRₛRₜ, (C1-10)alkoxy, cycloalkyl, aryl, or heteroaryl, which aryl or heteroaryl is optionally substituted with one or more groups independently selected from halo, hydroxy, (C1-10)alkyl, (C2-10)alkenyl, (C2-10)alkynyl, (C1-10)alkanoyl, (C1-10)alkoxy, (C1-10)alkanoyloxy, (C1-10)alkoxycarbonyl, NRₙRₚ, SRᵣ, S(O)Rᵣ, or S(O)₂Rᵣ;

R_{d} and Rₑ are each independently H or (C1-10)alkyl;

Z^{2b} is H, (C1-10)alkyl, (C2-10)alkenyl, or (C2-10)alkynyl;

Q¹ is (C1-10)alkyl, (C2-10)alkenyl, or (C2-10)alkynyl which Q¹ is optionally substituted with R⁴ or R_{c}; or Q¹ and Z^{2a} taken together with the atoms to which they are attached form a heterocycle, which heterocycle may optionally be substituted with one or more oxo (═O), R⁴, or A³;

each X is independently a bond, O, S, or NR³;

Y is a polycarbocycle or a polyheterocycle, which polycarbocycle or a polyheterocycle is optionally substituted with one or more R⁴, halo, carboxy, hydroxy, (C1-10)alkyl, (C2-10)alkenyl, (C2-10)alkynyl, (C1-10)alkanoyl, (C1-10)alkoxy, (C1-10)alkanoyloxy, (C1-10)alkoxycarbonyl, NRₙRₚ, SRᵣ, S(O)Rᵣ, or S(O)₂Rᵣ;

each R⁴ is independently —P(Y³)(OA²)(OA²), —P(Y³)(OA²)(N(A²)₂), —P(Y³)(A²)(OA²), —P(Y³)(A²)(N(A²)₂), or P(Y³)(N(A²)₂)(N(A²)₂);

each Y³ is independently O, S, or NR

each Rₙ, and Rₚ is independently H, (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, which (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, is optionally substituted with one or more R¹, halo, hydroxy, carboxy, cyano, or (C1-10)alkoxy; or Rₙ and Rₚ together with the nitrogen to which they are attached form a pyrrolidine, piperidine, piperazine, morpholino, or thiomorpholino ring;

each Rᵣ is independently H, (C1-10)alkyl, (C2-10)alkenyl, (C2-10)alkynyl, (C1-10)alkanoyl, heterocycle, or (C1-10)alkoxycarbonyl, wherein any (C1-10)alkyl, (C2-10)alkenyl, (C2-10)alkynyl, (C1-10)alkanoyl, heterocycle, or (C1-10)alkoxycarbonyl is optionally substituted with one or more A³;

each Rₛ and Rₜ is independently H, (C1-10)alkyl, (C2-10)alkenyl, (C2-10)alkynyl, (C1-10)alkanoyl, S(═O)₂A², (C1-10)alkoxy, (C1-10)alkanoyloxy, or (C1-10)alkoxycarbonyl, which (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, is optionally substituted with one or more R¹, halo, hydroxy, carboxy, cyano, or (C1-10)alkoxy; or Rₛ and Rₜ together with the nitrogen to which they are attached form a pyrrolidine, piperidine, piperazine, morpholino, or thiomorpholino ring wherein one or more carbon atoms of said pyrrolidine, piperidine, piperazine, morpholino or thiomorpholino ring is optionally replaced by S(═O), S(═O)₂, or C(═O);

Z^{2a} is H, (C1-10)alkyl, (C2-10)alkenyl, (C2-10)alkynyl, haloalkyl, (C1-10)alkyl-S(═O)₂—(C1-10)alkyl, or cycloalkyl, wherein any carbon atom of Z^{2a} may optionally be replaced with a heteroatom selected from O, S or N and wherein any cycloalkyl is optionally substituted with one or more (C1-10)alkyl, (C2-10)alkenyl, (C2-10)alkynyl, F, Cl, Br, or I; or Z^{2a} optionally forms a heterocycle with one or more R¹, R², Q¹, or A³;

each A³ is independently selected from PRT, H, —OH, —C(O)OH, cyano, alkyl, alkenyl, alkynyl, amino, amido, imido, imino, halogen, CF₃, —OCF₃, CH₂CF₃, cycloalkyl, nitro, aryl, aralkyl, alkoxy, aryloxy, heterocycle, —C(A²)₃, —C(A²)₂-C(O)A², —C(O)A², —C(O)OA², —O(A²), —N(A²)₂, —S(A²), —CH₂P(Y¹)(A²)(OA²), —CH₂P(Y¹)(A²)(N(A²)₂), —CH₂P(Y¹)(OA²)(OA²), —OCH₂P(Y¹)(OA²)(OA²), —OCH₂P(Y¹)(A²)(OA²), —OCH₂P(Y¹)(A²)(N(A²)₂), —C(O)OCH₂P(Y¹)(OA²)(OA²), —C(O)OCH₂P(Y¹)(A²)(OA²), —C(O)OCH₂P(Y¹)(A²)(N(A²)₂), —CH₂P(Y¹)(OA²)(N(A²)₂), —OCH₂P(Y¹)(OA²)(N(A²)₂), —C(O)OCH₂P(Y¹)(OA²)(N(A²)₂), —CH₂P(Y¹)(N(A²)₂)(N(A²)₂), —C(O)OCH₂P(Y)(N(A²)₂)(N(A²)₂), —OCH₂P(Y¹)(N(A²)₂)(N(A²)₂), —(CH₂)ₘ-heterocycle, —(CH₂)ₘC(O)Oalkyl, —O—(CH₂)ₘ—O—C(O)—Oalkyl, —O—(CH₂)ᵣ—O—C(O)—(CH₂)ₘ-alkyl, —(CH₂)ₘO—C(O)—O-alkyl, —(CH₂)ₘO—C(O)—O-cycloalkyl, —N(H)C(Me)C(O)O-alkyl, SRᵣ, S(O)Rᵣ, S(O)₂Rᵣ, Si(R³)₃, or alkoxy arylsulfonamide,

wherein each A³ may be optionally substituted with

1 to 4 —R¹, —P(Y¹)(OA²)(OA²), —P(Y¹)(OA²)(N(A²)₂), —P(Y¹)(A²)(OA²), —P(Y¹)(A²)(N(A²)₂), or P(Y¹)(N(A²)₂)(N(A²)₂), —C(═O)N(A²)₂), halogen, alkyl, alkenyl, alkynyl, aryl, carbocycle, heterocycle, aralkyl, aryl sulfonamide, aryl alkylsulfonamide, aryloxy sulfonamide, aryloxy alkylsulfonamide, aryloxy arylsulfonamide, alkyl sulfonamide, alkyloxy sulfonamide, alkyloxy alkylsulfonamide, arylthio, —(CH₂)ₘheterocycle, —(CH₂)ₘ—C(O)O-alkyl, —O(CH₂)ₘOC(O)Oalkyl, —O—(CH₂)ₘ—O—C(O)—(CH₂)ₘ-alkyl, —(CH₂)ₘ—O—C(O)—O-alkyl, (CH₂)ₘ—O—C(O)—O-cycloalkyl, —N(H)C(CH₃)C(O)O-alkyl, or alkoxy arylsulfonamide, optionally substituted with R¹;

optionally each independent instance of A³ and Q¹ can be taken together with one or more A³ or Q¹ groups to form a ring;

A² is independently selected from PRT, H, alkyl, alkenyl, alkynyl, amino, amino acid, alkoxy, aryloxy, cyano, haloalkyl, cycloalkyl, aryl, heteroaryl, heterocycle, alkylsulfonamide, or arylsulfonamide, wherein each A² is optionally substituted with A³;

R^{f} is H, alkyl, alkenyl, alkynyl, aryl, heteroaryl, or cycloalkyl, which R^{f} is optionally substituted with one or more R_{g};

each R_{g} is independently alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;

each Rₕ and Rᵢ is independently H, alkyl, or haloalkyl;

m is 0 to 6;

Z¹ is -L¹-A⁴-L²-A⁵;

L¹ is a bond, (C1-10)alkyl, O, S, —C(═O)—, —C(═O)O—, —OC(═O)—, —C(═O)NR³—, —NR³C(═O)—, —S(O)—, —S(O)₂—, —NR S(O)₂—, —S(O)₂NR³—, or NR³;

A⁴ is a monocyclic heteroaryl containing 1, 2, or 3 N, which A⁴ is optionally substituted with one or more A³;

L² is (C1-10)alkyl, O, S, —C(═O)—, —C(═O)O—, —OC(═O)—, —C(═O)NR³—, —NR³C(═O)—, —S(O)—, —S(O)₂—, —NR³S(O)₂—, —S(O)₂NR³—, or NR³; and

A⁵ is aryl, alkyl, cycloalkyl, or heteroaryl, which A⁵ is optionally substituted with one or more A³.

### Specific Embodiment 2

In one specific embodiment the invention provides the compound of specific embodiment 1 wherein R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

### Specific Embodiment 3

In one specific embodiment the invention provides the compound of specific embodiment 1 wherein R^{f} is cyclopropyl.

### Specific Embodiment 4

In one specific embodiment the invention provides the compound of specific embodiment 1 wherein R^{f} is 1-methylcyclopropyl.

### Specific Embodiment 5

In one specific embodiment the invention provides the compound of any one of specific embodiments 1-4 which is a compound of formula (II):

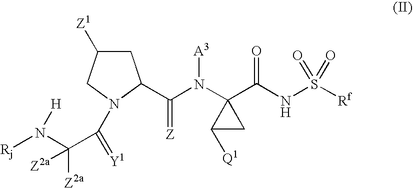

or a pharmaceutically acceptable salt, or prodrug thereof, wherein: Rⱼ is tert-butoxycarbonyl, cyclopentyloxycarbonyl, 2,2,2-trifluoro-1,1-dimethylethyl, 1-methylcyclopropyloxycarbonyl, 2-(N,N-dimethylamino)-1-1-dimethylethoxycarbonyl, 2-morpholino-1-1-dimethylethoxycarbonyl, tetrahydrofur-3-yloxycarbonyl, or

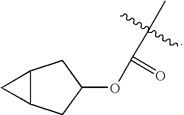

### Specific Embodiment 6

In one specific embodiment the invention provides the compound of specific embodiment 5 wherein Z is O; Y¹ is O; and one of Z^{2a} and Z^{2b} is hydrogen.

### Specific Embodiment 7

In one specific embodiment the invention provides the compound of any one of specific embodiments 1-6 wherein Q¹ is vinyl, ethyl, cyanomethyl, propyl, 2-fluoroethyl, 2,2-difluoroethyl, or 2-cyanoethyl.

### Specific Embodiment 8

In one specific embodiment the invention provides the compound of specific embodiment 1 wherein Q¹ and Z^{2a} taken together with the atoms to which they are attached form a 12-18 membered heterocycle, which heterocycle may optionally be substituted with one or more oxo (═O) or A³.

### Specific Embodiment 9

In one specific embodiment the invention provides the compound of any one of specific embodiments 1-4 which is a compound of formula (III):

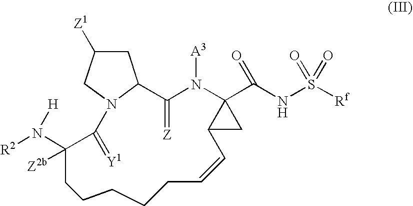

or a pharmaceutically acceptable salt, or prodrug thereof.

### Specific Embodiment 10

In one specific embodiment the invention provides the compound of any one of specific embodiments 1-4 which is a compound of formula (IV):

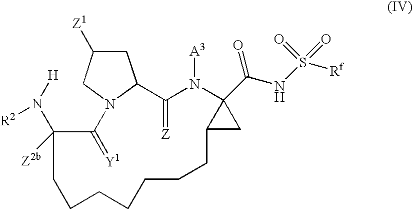

or a pharmaceutically acceptable salt, or prodrug thereof.

### Specific Embodiment 11

In one specific embodiment the invention provides the compound of specific embodiment 1 wherein Z^{2a} is tert-butyl, 1-methylcyclohexyl, tetrahydropyran-4-yl, 1-methylcyclohexyl, 4,4-difluorocyclohexyl, 2,2,2-trifluoro-1-trifluoromethylethyl, or cyclopropyl.

### Specific Embodiment 12

In one specific embodiment the invention provides the compound of specific embodiment 1 wherein X is O, S, or NR³.

### Specific Embodiment 13

In one specific embodiment the invention provides the compound of specific embodiment 1 wherein X is O,

### Specific Embodiment 14

In one specific embodiment the invention provides the compound of specific embodiment 1 wherein Y is a polycarbocycle.

### Specific Embodiment 15

In one specific embodiment the invention provides the compound of specific embodiment 1 wherein Y is polyheterocycle.

### Specific Embodiment 16

In one specific embodiment the invention provides the compound of specific embodiment 1 wherein Y is a fused carbocyclic ring system.

### Specific Embodiment 17

In one specific embodiment the invention provides the compound of specific embodiment 1 wherein Y is a fused heterocyclic ring system.

### Specific Embodiment 18

In one specific embodiment the invention provides the compound of specific embodiment 1 wherein Y is a fused carbocyclic ring system comprising one or more double bonds.

### Specific Embodiment 19

In one specific embodiment the invention provides the compound of specific embodiment 1 wherein Y is a fused heterocyclic ring system comprising one or more double bonds.

### Specific Embodiment 20

In one specific embodiment the invention provides the compound of specific embodiment 1 wherein Y is a bridged carbocyclic ring system.

### Specific Embodiment 21

In one specific embodiment the invention provides the compound of specific embodiment 1 wherein Y is a bridged heterocyclic ring system.

### Specific Embodiment 22

In one specific embodiment the invention provides the compound of specific embodiment 1 wherein Y is a bridged carbocyclic ring system comprising one or more double bonds.

### Specific Embodiment 23

In one specific embodiment the invention provides the compound of specific embodiment 1 wherein Y is a bridged heterocyclic ring system comprising one or more double bonds.

### Specific Embodiment 24

In one specific embodiment the invention provides the compound of specific embodiment 1 wherein Y comprises a bridged ring system selected from:

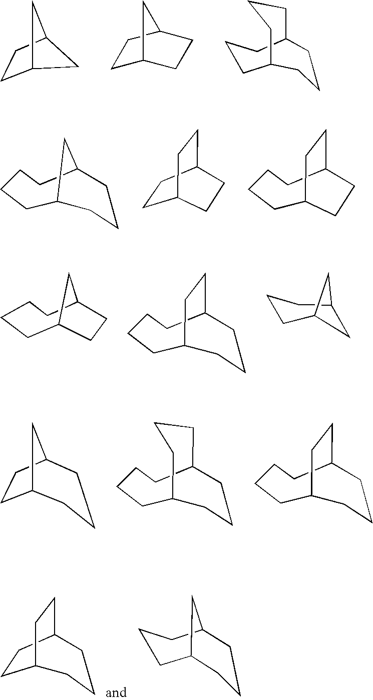

wherein one or more carbon atoms in the bridged ring system is optionally replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo; and wherein the ring system optionally comprises one or more double bonds.

### Specific Embodiment 25

In one specific embodiment the invention provides the compound of specific embodiment 24 wherein the ring system comprises one or more double bonds.

### Specific Embodiment 26

In one specific embodiment the invention provides The compound of specific embodiment 24 wherein one or more carbon atoms in the bridged ring system is replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo.

### Specific Embodiment 27

In one specific embodiment the invention provides the compound of specific embodiment 1 wherein Y comprises a fused ring system selected from:

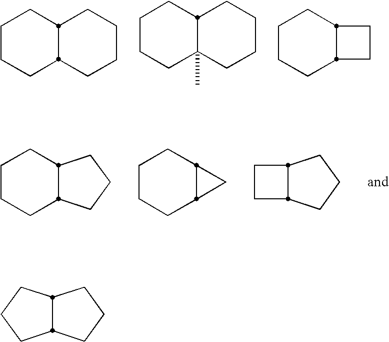

wherein one or more carbon atoms in the fused ring system is optionally replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo; and wherein the ring system optionally comprises one or more double bonds.

### Specific Embodiment 28

In one specific embodiment the invention provides the compound of specific embodiment 27 wherein one or more carbon atoms in the bridged ring system is replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo.

### Specific Embodiment 29

In one specific embodiment the invention provides the compound of specific embodiment 1 wherein Y is selected from:

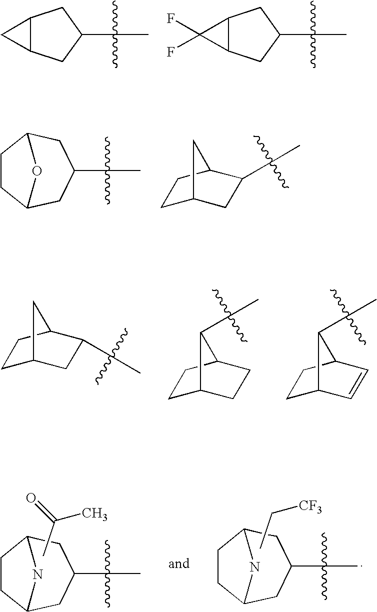

### Specific Embodiment 30

In one specific embodiment the invention provides the compound of any one of specific embodiments 1-29 wherein L¹ is O.

### Specific Embodiment 31

In one specific embodiment the invention provides the compound of any one of specific embodiments 1-30 wherein A⁴ is a pyrimidine or triazine ring that is optionally substituted with one or more A³.

### Specific Embodiment 32

In one specific embodiment the invention provides the compound of specific embodiment 31 wherein each A³ is independently selected from halogen, aryl, heterocycle, —N(A²)₂, or SRᵣ wherein each A³ may be optionally substituted with 1 to 4-R¹.

### Specific Embodiment 33

In one specific embodiment the invention provides the compound of any one of specific embodiments 1-30 wherein A⁴ is a pyrimidinyl or triazinyl ring that is optionally substituted with one or more chloro, pyrrolidinyl, piperidinyl, morpholino, 2-hydroxyethylamino, dimethylamino, isopropylamino, piperazinyl, cyclopentylamino, imidazolyl, 1,2,4-triazolyl, phenyl, N-(trimethylsilylmethyl)amino, 1,3-thiazol-2-yl, methylthio, 4-fluorophenylamino, methoxy, or

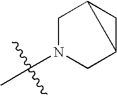

### Specific Embodiment 34

In one specific embodiment the invention provides the compound of any one of specific embodiments 1-33 wherein L² is a O, S, or NR³.

### Specific Embodiment 35

In one specific embodiment the invention provides the compound of any one of specific embodiments 1-34 wherein A⁵ is a phenyl, tetrazolyl, thiadiazolyl, cyclopentyl, or thiazolyl ring that is optionally substituted with one or more A³.

### Specific Embodiment 36

In one specific embodiment the invention provides the compound of specific embodiment 35 wherein each A³ is independently fluoro, methyl, trifluoromethyl, or trifluoromethoxy.

### Specific Embodiment 37

In one specific embodiment the invention provides the compound of any one of specific embodiments 1-33 wherein -L²-A⁵ is selected from:

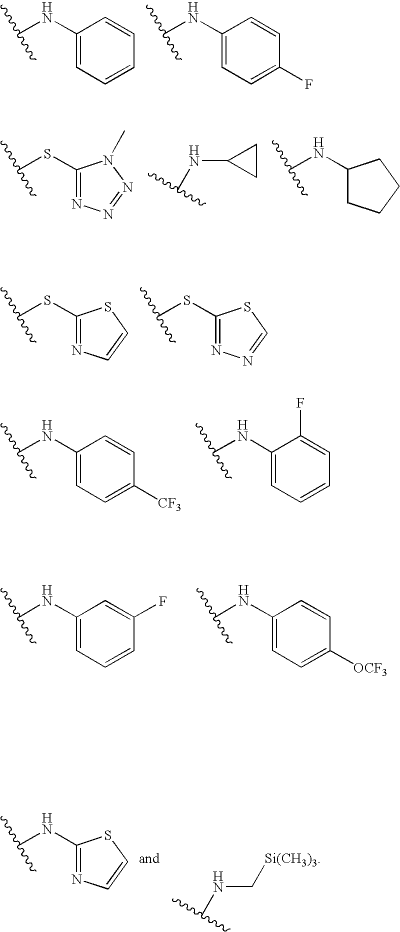

### Specific Embodiment 38

In one specific embodiment the invention provides the compound of any one of specific embodiments 1-29 wherein Z¹ is selected from:

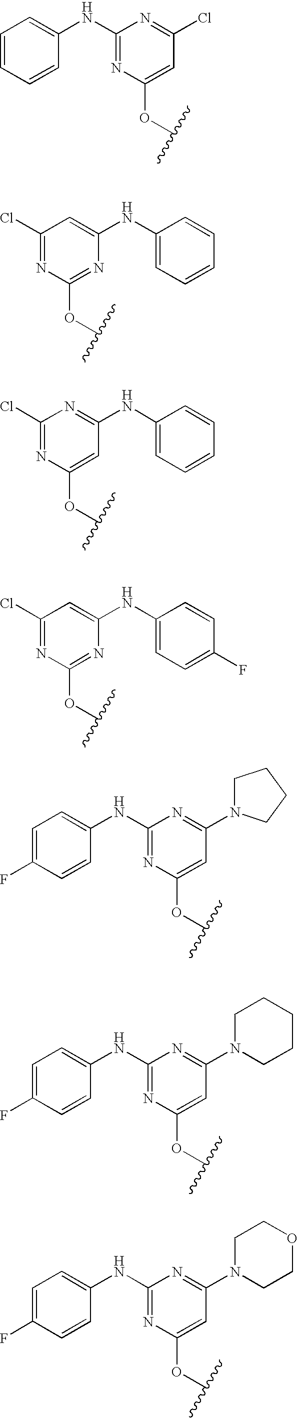

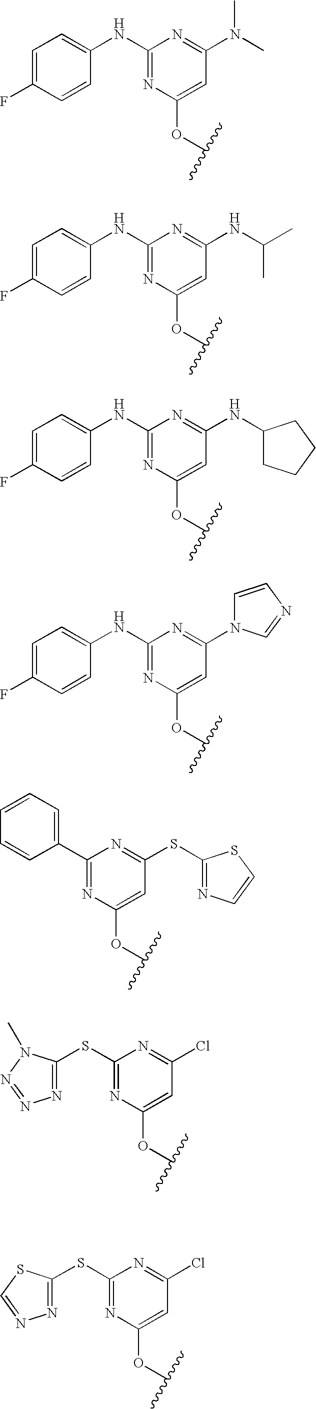

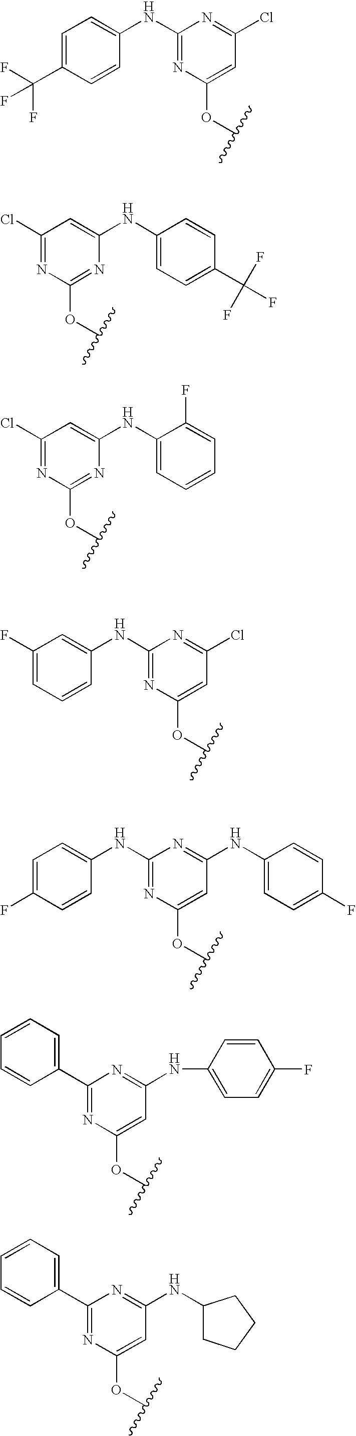

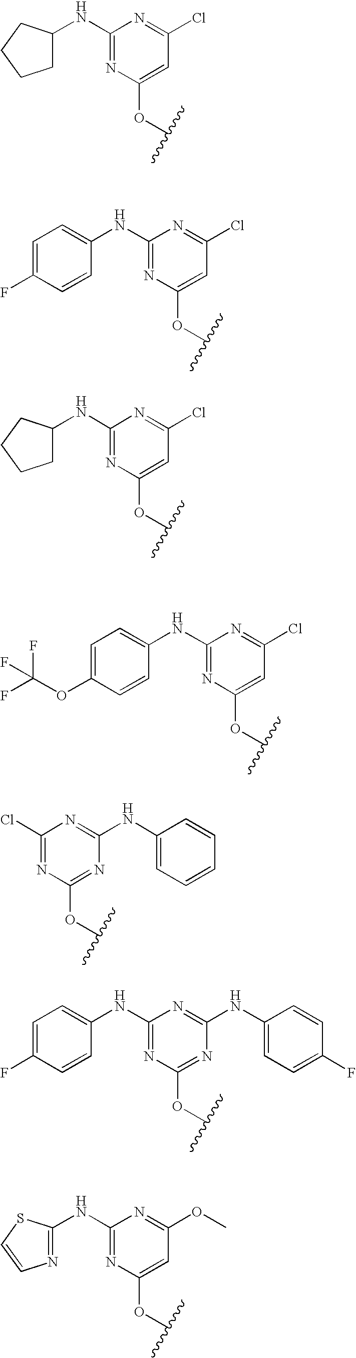

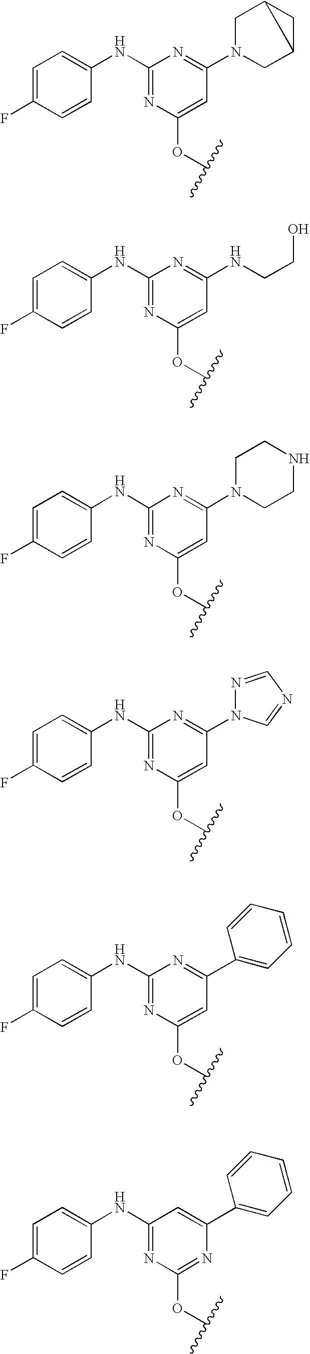

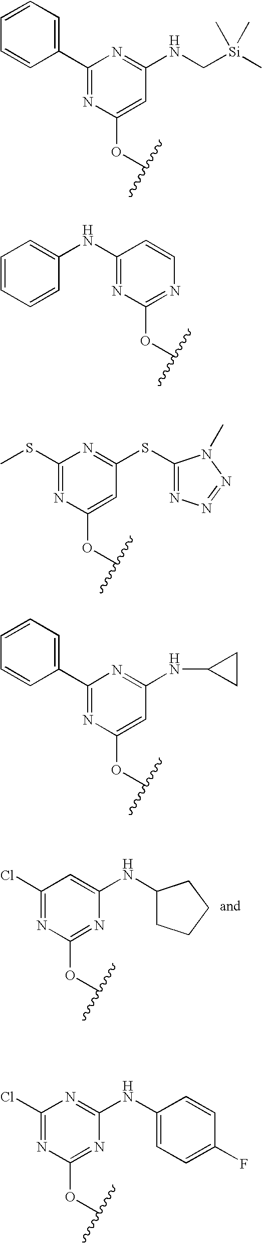

### Specific Embodiment 39

In one specific embodiment the invention provides the compound of any one of specific embodiments 1-35 wherein A⁵ is aryl, (C₂-C₁₀)alkyl, cycloalkyl, or heteroaryl, which A⁵ is optionally substituted with one or more A³.

### Specific Embodiment 40

In one specific embodiment the invention provides the compound of any one of specific embodiments 1-35 wherein A⁵ is aryl, cycloalkyl, or heteroaryl, which A⁵ is optionally substituted with one or more A³.

### Specific Embodiment 41

In one specific embodiment the invention provides the compound of any one of specific embodiments 1-35 wherein A⁵ is alkyl or cycloalkyl substituted with one or more A³.

### Specific Embodiment 42

In one specific embodiment the invention provides the compound of any one of specific embodiments 1-35 wherein A⁵ is alkyl or cycloalkyl substituted with Si(R³)₃.

### SCHEMES AND EXAMPLES

General aspects of these exemplary methods are described below and in the Examples. Each of the products of the following processes is optionally separated, isolated, and/or purified prior to its use in subsequent processes.

A number of exemplary methods for the preparation of compounds of the invention are provided herein, for example, in the Examples hereinbelow. These methods are intended to illustrate the nature of such preparations are not intended to limit the scope of applicable methods. Certain compounds of the invention can be used as intermediates for the preparation of other compounds of the invention.

### Examples

### Example 1

### Preparation of Intermediates

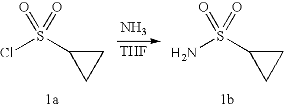

Ammonia gas was bubbled through THF (355 mL) at 0° C. for 20 min. Neat cyclopropylsulfonyl chloride (1a, 15 g, 0.11 mol) was dropwise added to the solution. The resulting solution was allowed to warm to room temperature and stirred for 17 h. The resulting suspension was filtered through a plug of silica gel, eluting with ethyl acetate. The filtrate was concentrated in vacuo to afford 11.5 g (89%) of cyclopropylsulfonamide 1b. ¹H NMR (300 MHz, CD₃OD): δ 2.63-2.53 (m, 1H), 1.09-0.95 (m, 4H).

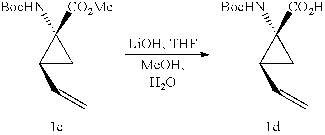

The vinylcyclopropyl ester 1c (9.23 g, 38.3 mmol) was dissolved in THF (127 mL) and MeOH (127 mL). Aqueous lithium hydroxide solution (1.5 N, 127 mL, 202 mmol) was added at a fast dropwise pace. After 2 h at room temperature, more lithium hydroxide (4.6 g, 202 mmol) was added, and the suspension was stirred at room temperature for an additional 17 h. The suspension was cooled to 0° C. and acidified to pH 5 with 1N HCl, whereupon ethyl acetate (300 mL) was added. It was further acidified to pH 1 and extracted with ethyl acetate (2×300 mL). The combined organic layers were washed with brine (200 mL), dried over magnesium sulfate, and concentrated in vacuo to afford 8.91 g of the acid 1d in quantitative yield.

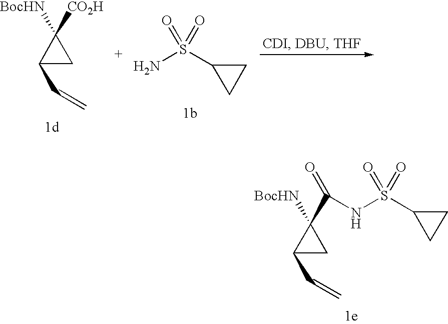

A solution of the acid 1d (8.70 g, 38.3 mmol) and carbonyldiimidazole (8.0 g, 50 mmol) in THF (125 mL) was refluxed for 1 h. To a slurry of cyclopropylsulfonamide 1b (6.03 g, 49.8 mmol) in THF (20 mL) was added at room temperature the above solution via cannular, followed by DBU (8.3 mL, 55.5 mmol). The mixture was stirred for 17 h whereupon it was acidified to pH 7 with 1N HCl. The organic solvent was removed in vacuo and ethyl acetate (200 mL) was added. The aqueous layer was further acidified to pH 1, and the organic layer drawn off. The aqueous layer was extracted with ethyl acetate (2×150 mL) and the combined organic layers were washed with brine (110 mL), dried over sodium sulfate, and concentrated in vacuo. The crude solid was purified by silica gel chromatography (ethyl acetate/hexanes) to afford 11.0 g of the product 1e in 85% yield. ¹H NMR (300 MHz, CD₃OD): δ 5.64-5.52 (m, 1H), 5.29 (d, J=17.1 Hz, 1H), 5.12 (d, J=10.5 Hz, 1H), 2.95 (bs, 1H), 2.29-2.15 (m, 1H), 1.88-1.82 (m, 1H), 1.47 (s, 9H), 1.33-1.00 (m, 5H).

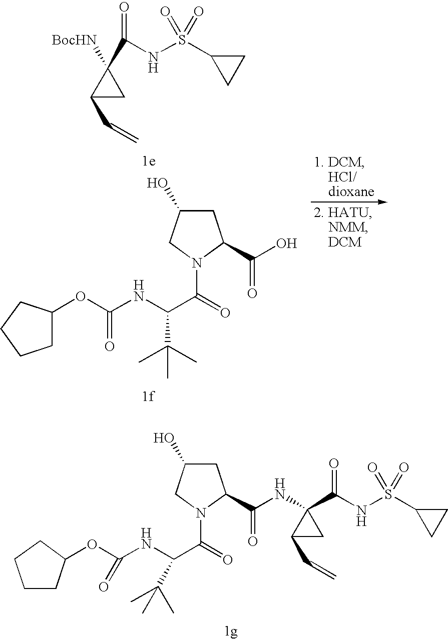

To the vinylcyclopropyl sulfonamide 1e (6.96 g, 21.1 mmol) in dichloromethane (53 mL) was added HCl/dioxane (4N, 53 mL). After 1.8 h at room temperature the volatiles were removed in vacuo and replaced with dichloromethane (80 mL), the 4-hydroxyproline acid 1f (3.43 g, 9.63 mmol), and HATU (6.22 g, 16.4 mmol). The resulting suspension was cooled to 0° C., and N-methylmorpholine (4.5 mL, 40.8 mmol) was added at a fast dropwise pace. The mixture was slowly warmed to room temperature and allowed to stir for an additional 16 h, whereupon dichloromethane (200 mL) was added. The organic layer was washed with HCl (0.5 N, 140 mL), brine (100 mL), dried over magnesium sulfate, and concentrated in vacuo. The crude residue was purified by silica gel chromatography (ethyl acetate/hexanes) to afford 3.79 g of 1g in 82% yield. ¹H NMR (300 MHz, CD₃OD): δ 6.94 (d, J=9.6 Hz, 1H), 5.85-5.70 (m, 1H), 5.32 (d, J=17.1 Hz, 1H), 5.14 (d, J=10.2 Hz, 1H), 5.03 (bs, 1H), 4.50 (bs, 1H), 4.42-4.32 (m, 2H), 3.90-3.79 (m, 2H), 3.00-2.87 (m, 1H), 2.25 (q, J=8.8 Hz, 1H), 2.18-2.08 (m, 1H), 2.06-1.95 (m, 1H), 1.93-1.51 (m, 9H), 1.48-1.35 (m, 1H), 1.34-1.17 (m, 2H), 1.15-1.02 (m, 2H), 1.02 (s, 9H); LCMS (M⁺+1): 568.99.

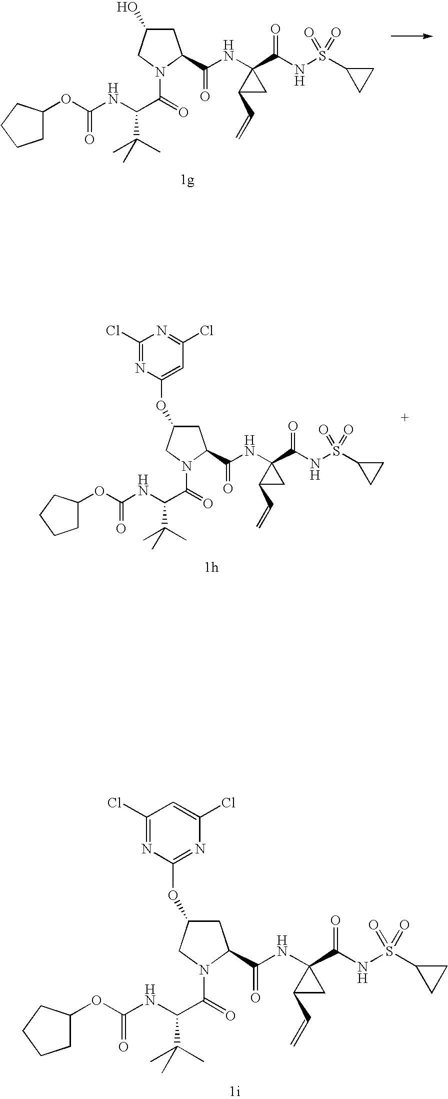

The intermediate 1g (5 g, 8.8 mmol) was dissolved in THF (33 mL) with 4N NaOH (11 mL) at room temperature, and stirred for 10 min. 2,4,6-Trichloroprimidine (4.88 g, 26.4 mmol) was added, and the reaction mixture was stirred at room temperature for 24 hours. The mixture was diluted with 10% MeOH/EtOAc (200 mL), washed with water plus brine (1:1, 100 mL). The organic layer was dried over sodium sulfate and concentrated. The residue was dissolved in dichloromethane, filtered through a pile of celite to remove the insoluble. The filtrate was purified by flash chromatography on a 330 g silica gel column with MeOH/CH₂Cl₂ (0-30%) to afford an isomeric mixture of the products 1h and 1i (6.16 g, 98%). LCMS (M⁺+1): 715.

### Example 2

### Preparation of Compounds 2a, 2b, 2c

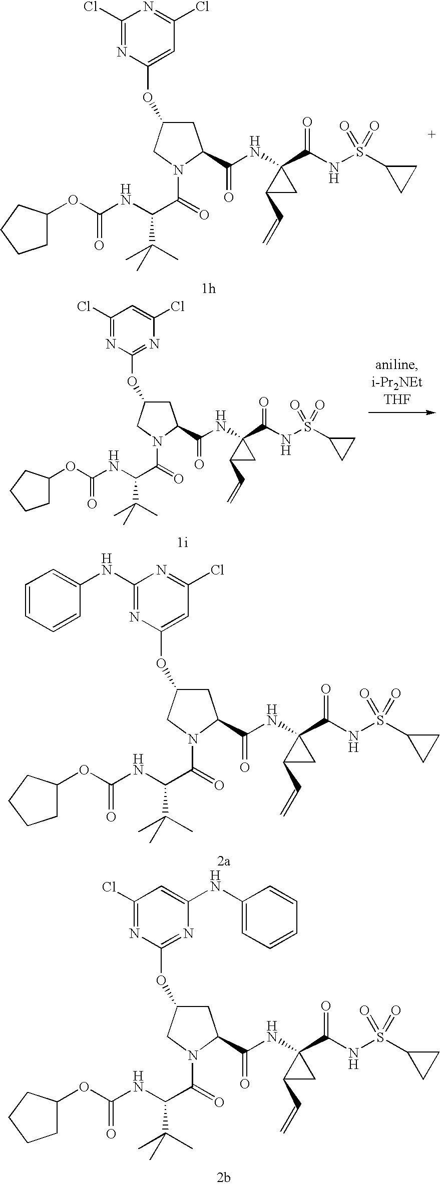

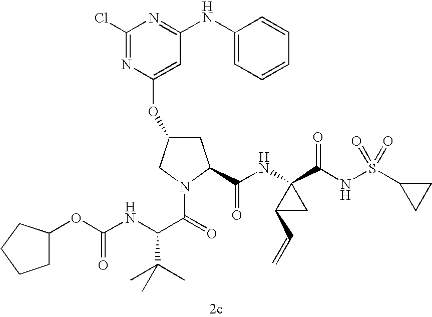

A solution of a mixture of 1h and 1i (999 mg, 1.40 mmol), aniline (0.51 mL, 5.59 mmol) and diisopropylethylamine (0.50 mL, 2.87 mmol) in THF (10 mL) was stirred at 60° C. for 23 h. After additional 0.51 mL (5.59 mmol) of aniline, and 0.50 mL (2.87 mmol) of diisopropylethylamine were added, the solution was stirred at 60° C. for 23 h and then refluxed for 22 h. After the solution was concentrated, the residue was dissolved in DMF and treated with 0.5 mL (6.49 mmol) of trifluoroacetic acid. The resulting crude products were purified by repeated preparative HPLC with water (0.05% TFA)—acetonitrile (0.05% TFA) as eluents to obtain 450 mg (42%) of 2a, 198 mg (18%) of 2b, and 55 mg (5%) of 2c. 2a: ¹H NMR (300 MHz, CD₃OD): δ 9.21 (s, 1H), 7.64 (d, 2H, J=8.4 Hz), 7.32 (t, 2H, J=7.8 Hz), 7.04 (t, 1H, J=7.5 Hz), 6.24 (s, 1H), 5.67-5.84 (m, 1H), 5.71 (br, 1H), 5.32 (d, 1H, J=17.1 Hz), 5.14 (d, 1H, J=10.2 Hz), 4.94 (br m, 1H), 4.43 (m, 1H), 4.24-4.34 (m, 2H), 4.43 (dd, 1H, J=11.1 and ˜3 Hz), 2.95 (m, 1H), 2.49 (dd, 1H, J=14.0 and 6.5 Hz), 2.17-2.30 (m, 2H), 1.89 (dd, 1H, J=8.1 and 5.4 Hz), 1.52-1.9 (m, 8H), 1.43 (dd, 1H, J=9.3 and 5.4 Hz), 1.22-1.32 (m, 2H), 1.07 (m, 2H), 1.02 (s, 9H). LC/MS=772 (M⁺+1). 2b: ¹H NMR (300 MHz, CD₃OD): δ 9.26 (s, 1H), 7.55 (d, 2H, J=8.1 Hz), 7.38 (t, 2H, J=8.0 Hz), 7.14 (t, 1H, J=7.5 Hz), 6.43 (s, 1H), 5.77 (dt, 1H, J=16.8 and ˜9.5 Hz), 5.60 (br, 1H), 5.32 (d, 1H, J=16.8 Hz), 5.14 (d, 1H, J=10.2 Hz), 4.9 (br m, 1H), 4.46 (dd, 1H, J=8.1 and 9.3 Hz), 4.26-4.36 (m, 2H), 4.01 (dd, 1H, J=9.3 and ˜3 Hz), 2.95 (m, 1H), 2.49 (dd, 1H, J=14.0 and 7.5 Hz), 2.15-2.32 (m, 2H), 1.89 (dd, 1H, J=7.2 and 6.3 Hz), 1.49-1.9 (m, 8H), 1.44 (dd, 1H, J=9.5 and 5.3 Hz), 1.21-1.29 (m, 2H), 1.08 (m, 2H), 1.02 (s, 9H). LC/MS=772 (M⁺+1). 2c: ¹H NMR (300 MHz, CD₃OD): δ 9.19 (s, 1H), 7.46 (d, 2H, J=˜8.1 Hz), 7.34 (t, 2H, J=˜7.8 Hz), 7.10 (t, 1H, J=˜7.5 Hz), 5.96 (s, 1H), 5.77 (dt, 1H, J=˜17.1 and ˜9.5 Hz), 5.63 (br, 1H), 5.31 (d, 1H, J=17.1 Hz), 5.13 (d, 1H, J=10.2 Hz), ˜4.9 (br m, 1H), 4.40 (dd, 1H, J=˜10.8 and ˜3 Hz), 4.17-4.3 (m, 2H), 4.43 (dd, 1H, J=˜11 and ˜3 Hz), 2.95 (m, 1H), 2.46 (dd, 1H, J=14.0 and 6.6 Hz), 2.14-2.30 (m, 2H), 1.88 (dd, 1H, J=8.4 and 5.4 Hz), 1.4-1.9 (m, 8H), 1.44 (dd, 1H, J=9.6 and 5.4 Hz), 1.2-1.32 (m, 2H), 1.07 (m, 2H), 1.03 (s, 9H). LC/MS=772 (M⁺+1).

### Example 3

### Preparation of Compounds 3a and 3b

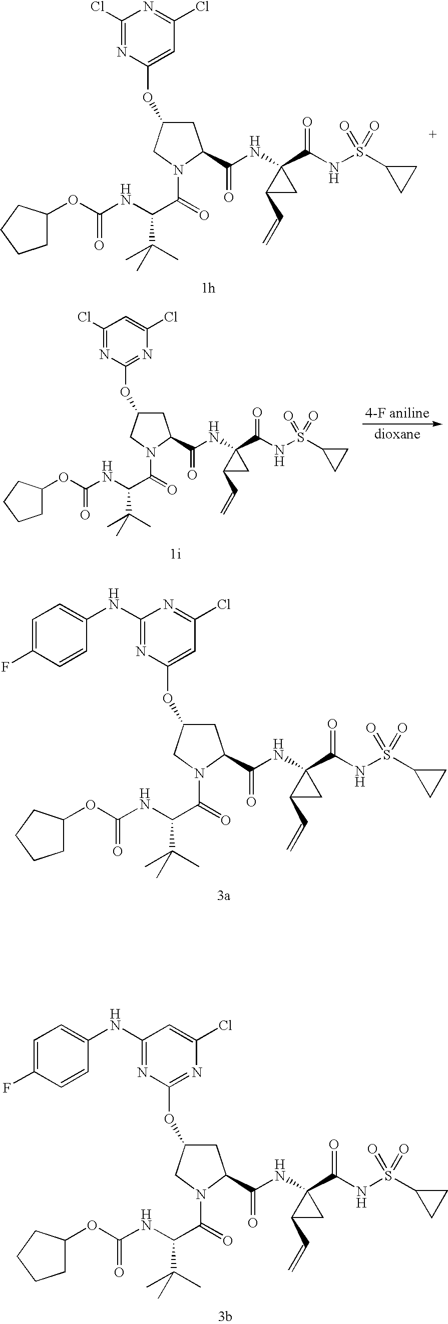

A solution of 1.0 g (1.40 mmol) of a mixture of 1h and 1i, 1.35 mL (14 mmol) of 4-fluoroaniline in dioxane (14 mL) was stirred at 120° C. for 6 h. After the solution was concentrated, the residue was dissolved in DMF and treated with 0.5 mL of trifluoroacetic acid. The resulting crude products were purified by repeated preparative HPLC with water (0.1% TFA)—acetonitrile (0.1% TFA) as eluents to obtain 500 mg (51%) of 3a and 150 mg (15%) of 3b. 3a: ¹H NMR (300 MHz, CD₃OD): δ 9.16 (s, 1H), 7.64 (m, 2H), 7.07 (t, 2H), 6.20 (s, 1H), 5.70-5.82 (m, 1H), 5.67 (br, 1H), 5.34-5.28 (d, 1H), 5.12-5.15 (d, 1H), 4.93 (br m, 1H), 4.43 (m, 1H), 4.23-4.26 (m, 2H), 4.03 (dd, 1H), 2.95 (m, 1H), 2.49 (dd, 1H), 2.20-2.25 (m, 2H), 1.89 (dd, 1H), 1.52-1.9 (m, 8H), 1.43 (dd, 1H), 1.22-1.32 (m, 2H), 1.07 (m, 2H), 1.02 (s, 9H). LC/MS=790.4 (M⁺+1); LC/MS Rₜ=3.03 min. 3b: LC/MS=790.4 (M⁺+1); LC/MS Rₜ=2.93 min.

### Example 4

### Preparation of Compound 4

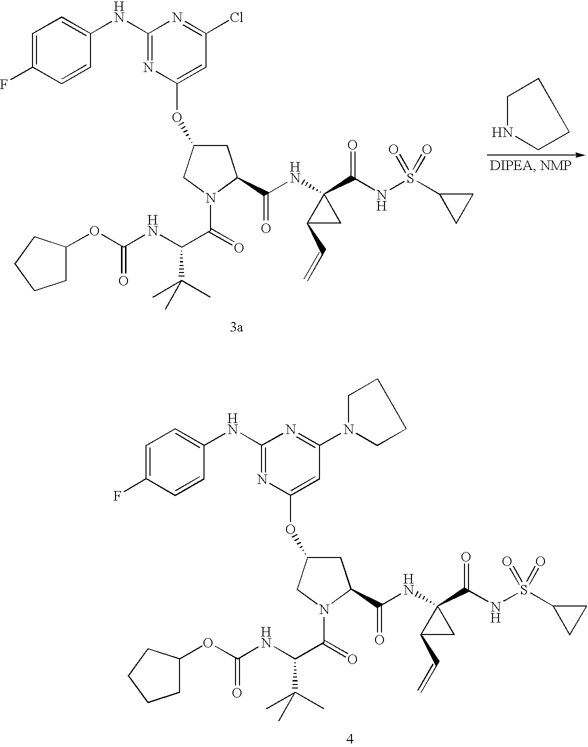

A mixture of the chloropyrimidine 3a (60 mg, 0.076 mmol), pyrrolidine (0.2 mL) and NMP (1 mL) was subjected to a microwave reactor for 10 min at 120° C. After removal of the volatile, the crude was purified by preparative HPLC using water (0.05% TFA) and acetonitrile (0.05% TFA) as eluents to give 46 mg (73%) of 4 as a white solid after lyophilization. ¹H NMR (300 MHz, CD₃OD): δ 7.71 (m, 2H), 6.99 (t, 2H), 5.83 (m, 1H), 5.60 (brl, 1H), 5.21 (s, 1H) overlapped with 5.20 (d, 1H), 5.03 (d, 1H), 4.45 (m, 1H), 3.9-4.3 (m, 3H), 3.44 (br s, 4H), 2.68 (m, 1H), 2.45 (m, 1H), 1.9-2.1 (m, 6H), 1.52-1.9 (m, 8H), 1.4-1.9 (m, 7H), 1.03 (s, 9H). MS (m/z) 825.4 [M+H]⁺, LC/MS Rₜ=3.07 min.

### Example 5

### Preparation of Compound 5

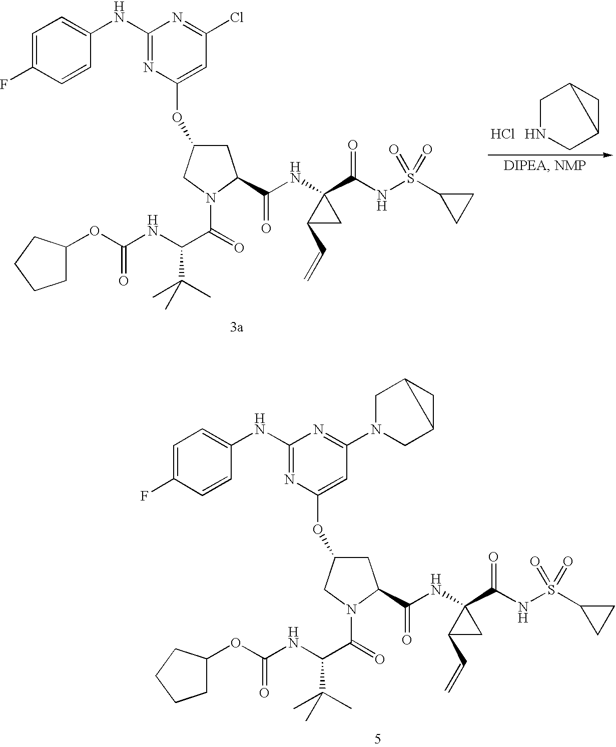

A solution of 70 mg (0.09 mmol) of the chloropyrimidine 3a, 190 mg (1.59 mmol) of 3-aza-bicyclo [3.1.0] hexane hydrochloride, and 0.34 mL (1.95 mmol) of diisopropylethylamine in NMP (1.9 mL) was heated at 120° C. for 1 h using a microwave reactor. The resulting mixture was acidified by adding trifluoroacetic acid to pH 5 and purified by preparative HPLC using water (0.05% TFA) and acetonitrile (0.05% TFA) as eluents to give 66 mg (88%) of 5 as a white solid after lyophilization. MS (m/z) 837.5 [M+H]⁺, LC/MS Rₜ=3.05 min. 3-aza-bicyclo [3.1.0] hexane hydrochloride was obtained according to Bioorg. Med. Chem. Lett. 15 (2005), 2093.

### Example 6

### Preparation of Compound 6

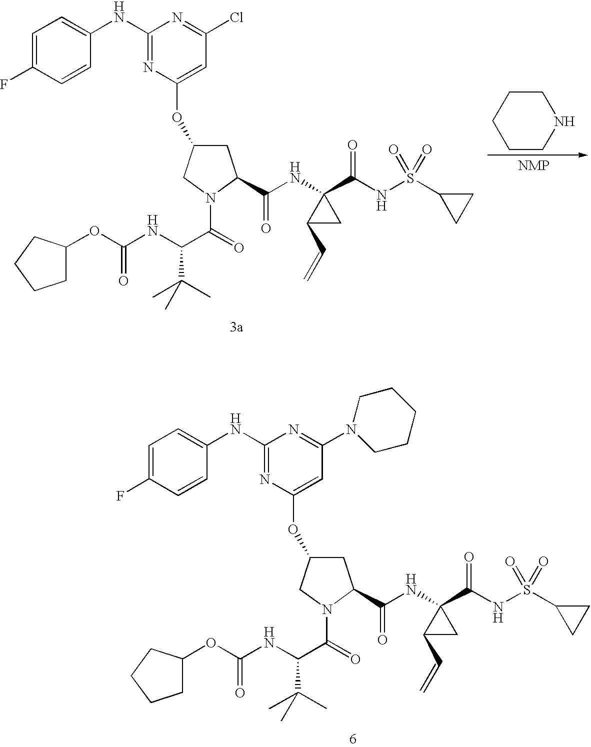

A solution of 18 mg (0.02 mmol) of the chloropyrimidine 3a, 60 μl (0.60 mmol) of piperidine in NMP (1 mL) was heated at 120° C. for 20 min using a microwave reactor. The resulting mixture was acidified by adding trifluoroacetic acid to pH 5 and purified by preparative HPLC using water (0.05% TFA) and acetonitrile (0.05% TFA) as eluents to give 14 mg (83%) of 6 as a white solid after lyophilization. MS (m/z) 839.6 [M+H]⁺, LC/MS Rₜ=3.11 min.

### Example 7

### Preparation of Compound 7

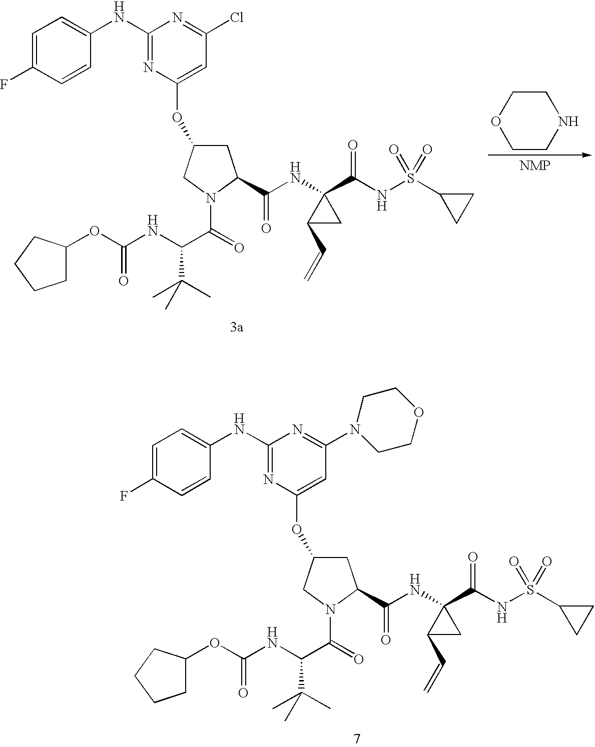

Compound 7 was prepared by following procedures similar to those described for compound 6 except using morpholine. MS (m/z) 841.5 [M+H]⁺, LC/MS Rₜ=2.90 min

### Example 8

### Preparation of Compound 8

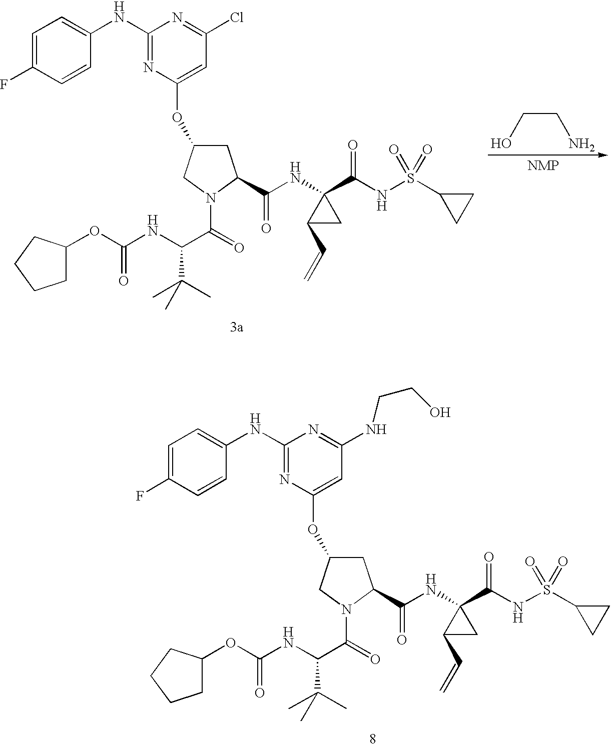

Compound 8 was prepared by following procedures similar to those described for compound 6 except using 2-aminoethanol. MS (m/z) 815.5 [M+H]⁺, LC/MS Rₜ=2.53 min.

### Example 9

### Preparation of Compound 9

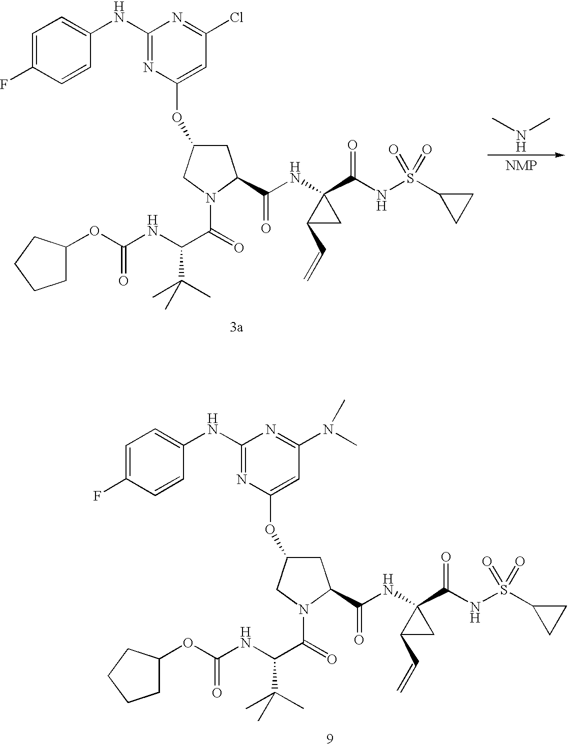

Compound 9 was prepared by following procedures similar to those described for compound 6 except using dimethylamine. MS (m/z) 799.5 [M+H]⁺, LC/MS Rₜ=2.93 min.

### Example 10

### Preparation of Compound 10

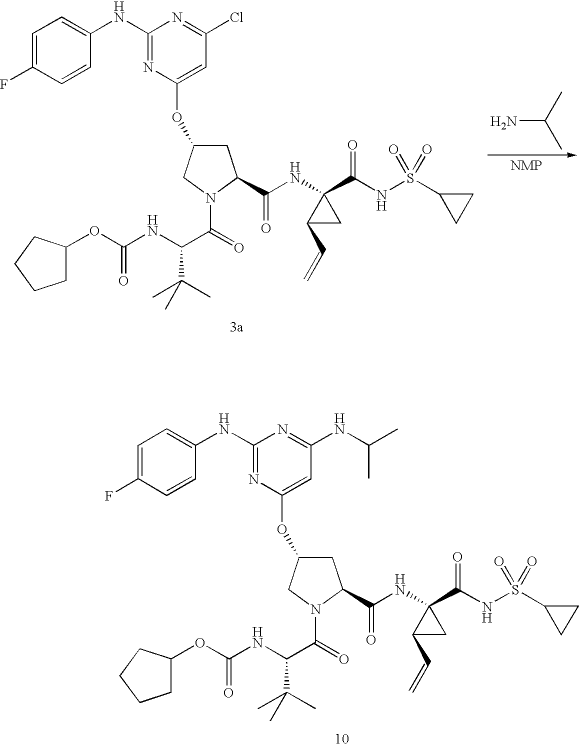

Compound 10 was prepared by following procedures similar to those described for compound 6 except using isopropylamine. MS (m/z) 813.6 [M+H]⁺, LC/MS Rₜ=2.85 min.

### Example 11

### Preparation of Compound 11

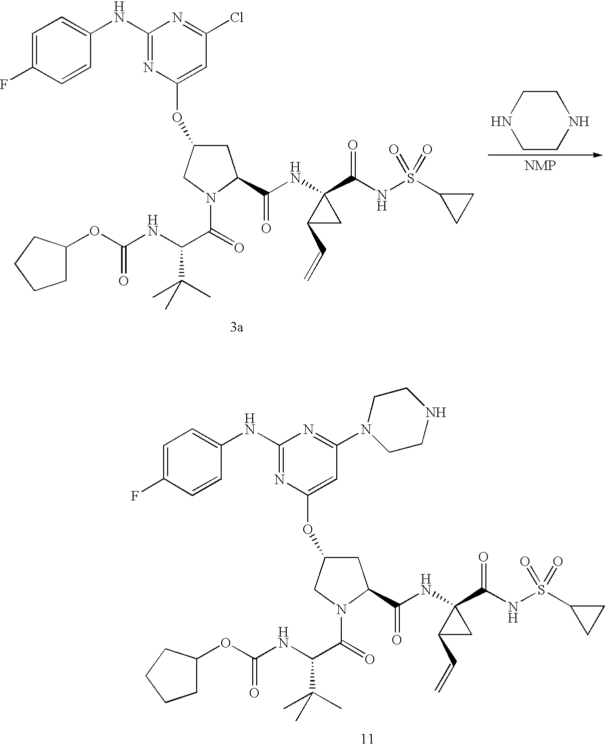

Compound 11 was prepared by following procedures similar to those described for compound 6 except using piperazine. MS (m/z) 840.5 [M+H]⁺, LC/MS Rₜ=2.09 min.

### Example 12

### Preparation of Compound 12

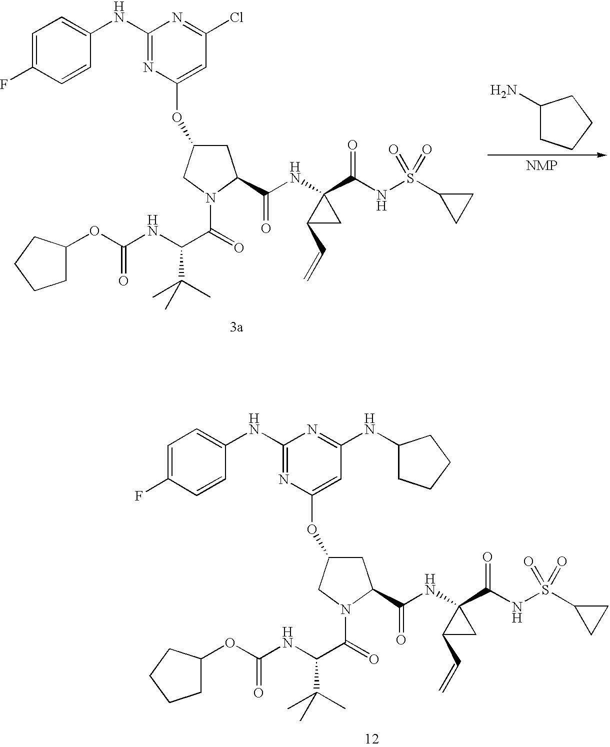

Compound 12 was prepared by following procedures similar to those described for compound 6 except using cyclopentylamine. MS (m/z) 839.5 [M+H]⁺, LC/MS Rₜ=2.96 min.

### Example 13

### Preparation of Compound 13

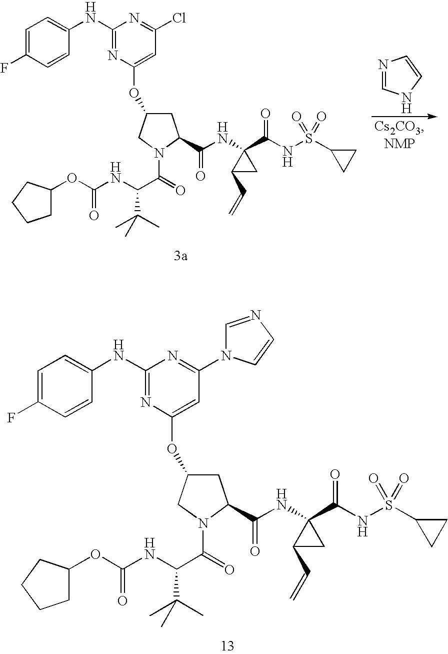

A mixture of 180 mg (0.24 mmol) of the chloropyrimidine 3a, 312 mg (4.56 mmol) of 1H-imidazole, 297 mg (0.91 mmol) of cesium carbonate in NMP (2.4 mL) was heated at 110° C. for 1 h using a microwave reactor. The resulting mixture was diluted with ethyl acetate (20 ml) and washed with 3% LiCl (2×20 ml), brine (20 ml) and concentrated in vacuo. The residue was purified by preparative HPLC using water (0.05% TFA) and acetonitrile (0.05% TFA) as eluents to give 60 mg (30%) of 13 as a white solid after lyophilization. MS (m/z) 822.5 [M+H]⁺, LC/MS Rₜ=2.57 min.

### Example 14

### Preparation of Compound 14

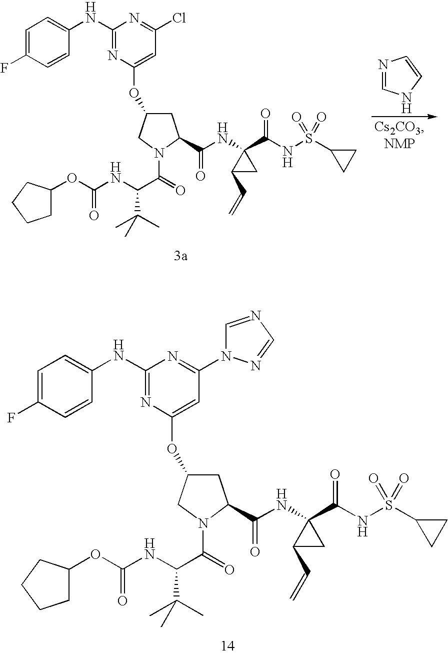

Compound 14 was prepared by following procedures similar to those described for compound 13 except using 1, 2, 4-triazole. MS (m/z) 823.5 [M+H]⁺, LC/MS Rₜ=2.82 min.

### Example 15

### Preparation of Compound 15

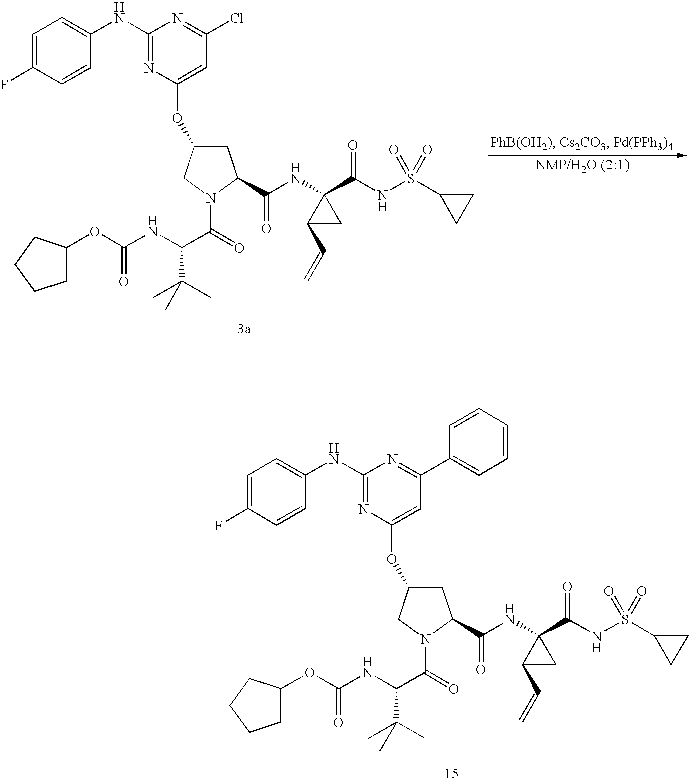

A mixture of 20 mg (0.02 mmol) of the chloropyrimidine 3a, 31 mg (0.25 mmol) of phenylboronic acid, 65 mg (0.2 mmol) of cesium carbonate and trace amount of tetrakis(triphenylphosphine)palladium in NMP (0.63 mL) and water (0.3 ml) was heated at 120° C. for 20 min using a microwave reactor. The resulting mixture was purified by preparative HPLC using water (0.05% TFA) and acetonitrile (0.05% TFA) as eluents to give 17 mg (82%) of 15 as a white solid after lyophilization. MS (m/z) 832.5 [M+H]⁺, LC/MS Rₜ=3.11 min.

### Example 16

### Preparation of Compound 16

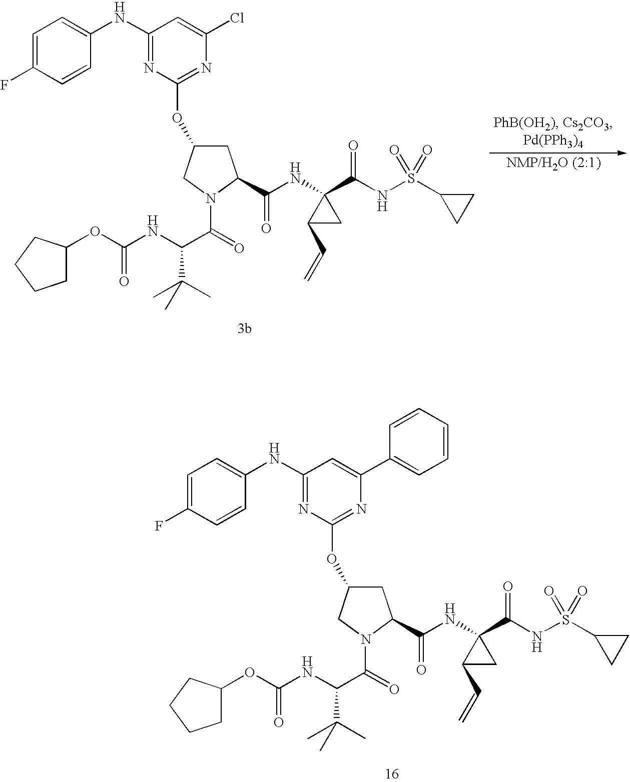

Compound 16 was prepared by following procedures similar to those described for compound 15 except using the chloropyrimidine 3b. MS (m/z) 832.5 [M+H]⁺, LC/MS Rₜ=2.92 min.

### Example 17

### Preparation of Compound 17

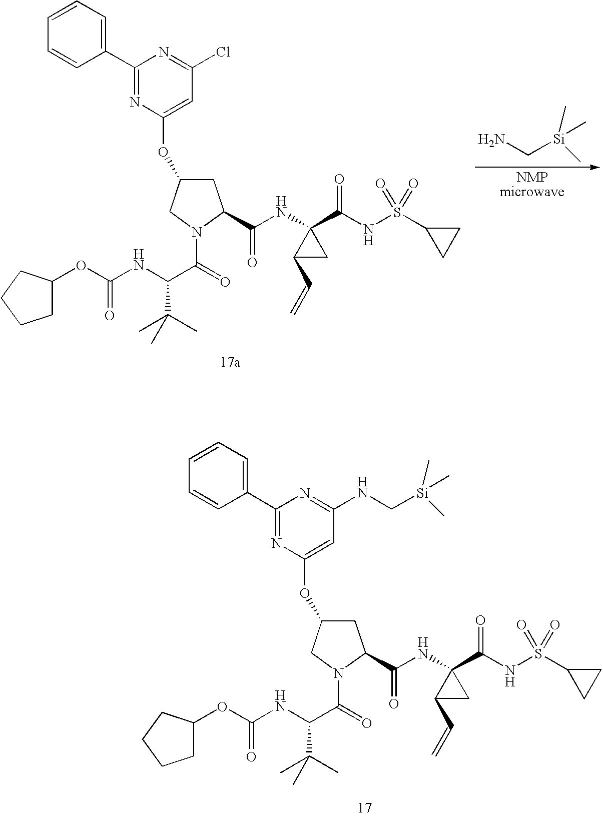

A mixture of the chloropyrimidine 17a (541 mg, 0.714 mmol) and (trimethylsilyl)methyl amine (0.96 ml, 7.14 mmol) in NMP (6 mL) in a sealed reaction vessel was subjected to a microwave reactor at 120° C. for 10 min. The crude product was purified by HPLC on an YMC-ODS preparative column to give the desired product 17 (480 mg, 82% yield). LC/MS Rₜ=2.86 min, m/z (+)=824 (M+1). Compound 17: ¹H NMR (300 MHz, CD₃OD): δ 9.21 (s, 1H), 8.23-8.21 (d, 2H), 7.62-7.53 (m, 3H), 6.02 (s, 1H), 5.82-5.70 (m, 2H), 5.33-5.28 (d, 1H), 5.12-5.15 (d, 1H), 4.80 (m, 1H), 4.50 (t, 1H), 4.39 (d, 1H), 4.25 (s, 1H), 4.07 (d, 1H), 2.94-2.97 (m, 2H), 2.58-2.56 (m, 1H), 2.34-2.20 (m, 2H), 1.90 (dd, 1H), 1.87-1.41 (m, 10H), 1.26-1.24 (m, 2H), 1.03-1.09 (m, 9H). 0.17 (s, 9H).

Compound 17a was obtained by following procedures similar to those described for compound 1h except using 4,6-dichloro-2-phenylpyrimidine.

### Example 18

### Preparation of Compound 18

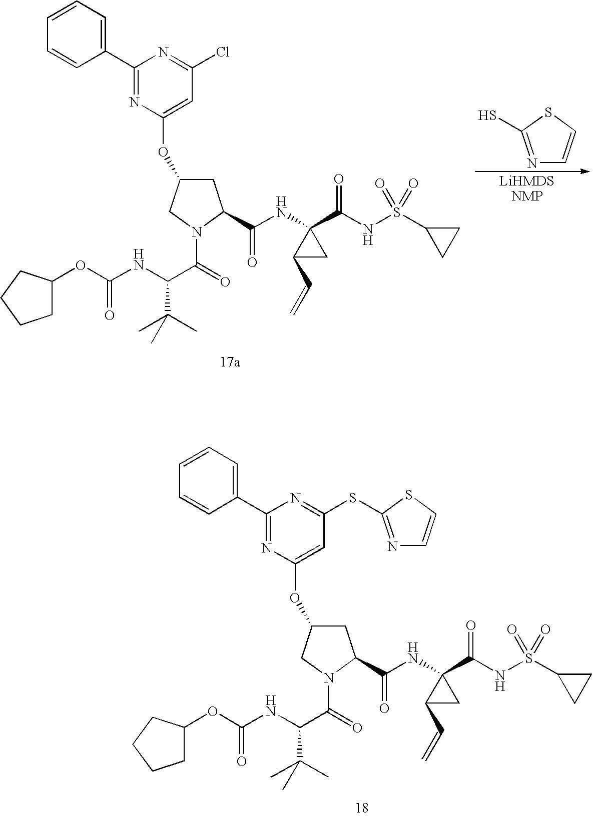

2-Mercaptothiazole (76.4 mg, 0.633 mmol) was dissolved in NMP (1 mL) at room temperature under N₂. LiHMDS (1M in THF, 0.57 ml, 0.57 mmol) was added dropwise into the above mixture. It was stirred at room temperature for 10 min. It was then heated to 60° C. for 30 min and allowed to cool down to room temperature again.

A solution of 17a (100 mg, 0.126 mmol) in 1 mL of NMP was added slowly into the reaction. The resulting mixture was stirred at room temperature for 2 h, at 60° C. for 2 h, then at 100° C. for 6 h. The reaction was complete. After cooling to room temperature, the crude mixture was purified by prep HPLC to give the desired product 18 (73 mg, 78% yield). LC/MS Rₜ=2.83 min, m/z (+)=838. Compound 18: ¹H NMR (300 MHz, CD₃OD): δ 9.19 (s, 1H), 8.34 (d, 2H), 8.04-7.95 (dd, 2H), 7.49-7.46 (m, 3H), 7.07 (t, 2H), 6.54 (s, 1H), 5.92 (s, 1H), 5.82-5.70 (m, 1H), 5.33-5.28 (d, 1H), 5.12-5.15 (d, 1H), 4.44 (m, 1H), 4.11-4.32 (m, 3H), 2.95 (m, 1H), 2.50 (dd, 1H), 2.33-2.20 (m, 2H), 1.90 (dd, 1H), 1.87-1.58 (m, 8H), 1.43 (dd, 1H), 1.26-1.24 (m, 2H), 1.07 (m, 2H), 1.02 (s, 9H).

### Example 19

### Preparation of Compound 19

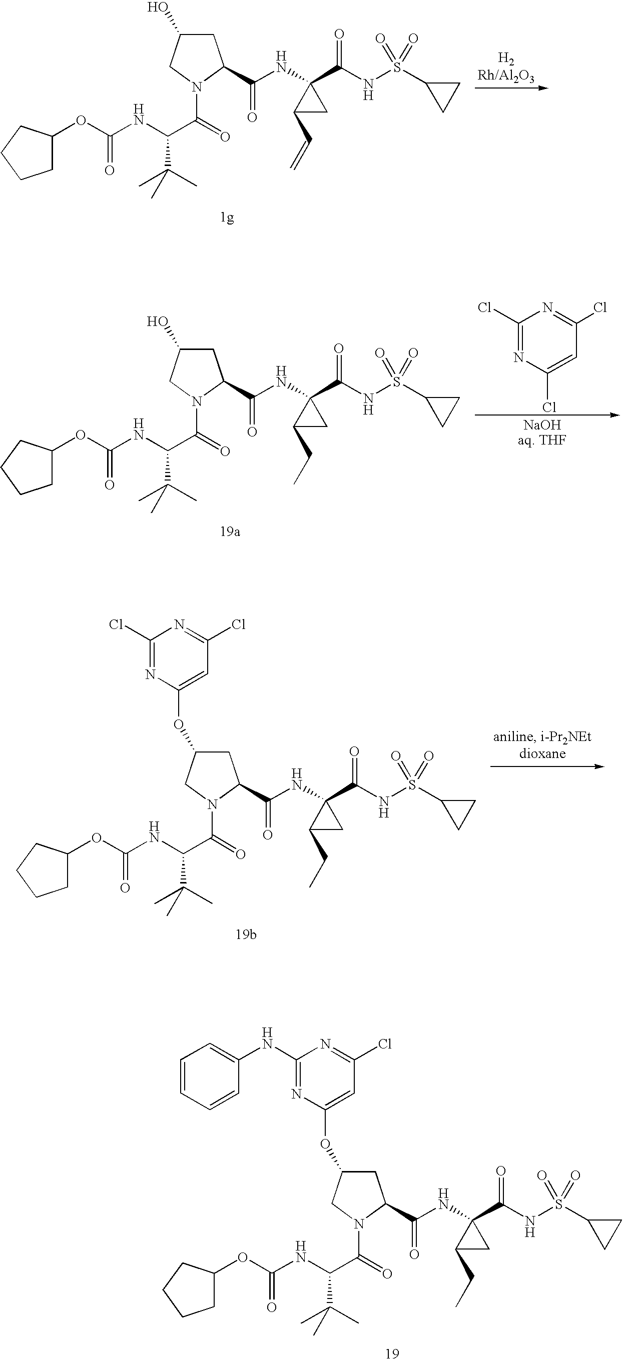

A mixture of the intermediate 1g (1.81 g, 3.17 mmol) and 181 mg of rhodium on alumina in ethyl acetate (24 mL) and methanol (12 mL) was vigorously stirred under H₂ atmosphere for 2 h. After additional 180 mg of rhodium on alumina was added, the mixture was stirred under H₂ atmosphere for 24 h and filtered through celite pad. The filtrate was concentrated and dried in vacuum to obtain 1.69 g of the crude 19a. LC/MS=571 (M⁺+1).

A mixture of 1.69 g (2.96 mmol) of 19a and 1.64 g (8.93 mmol) of 2,4,6-trichloropyrimidine in 4N NaOH (3.7 mL) and THF (11.1 mL) was vigorously stirred at rt for 18 h. After the mixture was diluted with ethyl acetate, it was washed with water (×2) and brine (×1), dried (MgSO₄), and filtered through celite. After the filtrate was concentrated, the residue was dissolved in CH₂Cl₂ and the insoluble material was removed by filtration through celite. After the removal of insoluble material was repeated several times, the residue was dissolved in ethyl acetate, filtered through celite, and the resulting solution was purified by combiflash chromatography to obtain 850 mg of the dichloropyrimidine 19b (which contains ˜35% of the other pyrimidine isomer). LC/MS=717 (M⁺+1).

A solution of 203.4 mg (0.283 mmol) of the dichloropyrimidine (19b), 0.26 mL (2.85 mmol) of aniline, and 0.25 mL (1.44 mmol) of diisopropylethylamine in 3 mL of dioxane was stirred at 120° C. bath for 10 h. The solution was filtered and purified by preparative HPLC to obtain 121 mg (55%) of compound 19 after lyophilization. ¹H NMR (300 MHz, CD₃OD): δ 9.12 (s, 1H), 7.64 (d, 2H, J=7.8 Hz), 7.32 (t, 2H, J=˜7.8 Hz), 7.04 (t, 1H, J=˜7.1 Hz), 6.23 (s, 1H), 5.70 (br, 1H), 4.94 (br, 1H), 4.43 (dd, 1H, J=˜9.6 and ˜7.5 Hz), 4.16-4.34 (m, 2H) 4.02 (dd, 1H, J=˜11.7 and ˜2 Hz), 2.97 (m, 1H), 2.47 (dd, 1H, J=14.1 and 6.9 Hz), 2.21 (m, 1H), 1.4-1.9 (m, 12H), 1.15-1.4 (m, 4H), 0.9-1.15 (m, 4H), 1.02 (s, 9H). LC/MS=774 (M⁺+1).

### Example 20

### Preparation of Compound 20

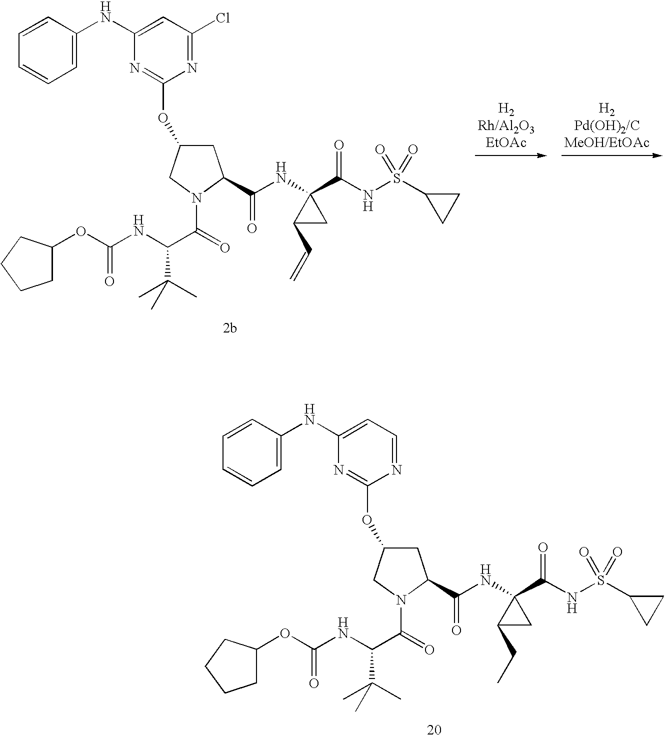

A mixture of 20.9 mg (0.027 mmol) of compound 2b and 10.2 mg of rhodium on alumina in 2 mL of ethyl acetate was stirred at room temperature under H₂ atmosphere for 23 h and the mixture was filtered through celite. After the filtrate was concentrated, the residue was dissolved in ethyl acetate (2 mL) and ˜2 mg of 10% Pd/C was added before the mixture was stirred under H₂ atmosphere for 1 h. To the reaction mixture was added additional Pd(OH)₂/C repeatedly and stirred under H₂ atmosphere until the reaction was complete. The mixture was diluted with methanol (3 mL), stirred under H₂ atmosphere for 40 min, and filtered through celite. The filtrate was concentrated and the residue was purified by preparative HPLC purifications using water (0.05% TFA) and acetonitrile (0.05% TFA) as eluents to afford 6.2 mg of compound 20 after lyophilization. ¹H NMR (300 MHz, CD₃OD): δ 9.12 (s, 1H), 8.03 (d, 1H, J=7.0 Hz), 7.42-7.8 (m, 4H), 7.27-7.42 (m, 1H), 6.63 (d, 1H, J=7.0 Hz), 5.74 (br, 1H), 4.94 (br, 1H), 4.3-4.54 (m, 2H), 4.21 (s, 1H), 4.01 (m, 1H), 2.97 (m, 1H), 2.55 (m, 1H), 2.27 (m, 1H), 1.45-0.9 (m, 20H), 1.03 (s, 9H). LC/MS=740 (M⁺+1).

### Example 21

### Preparation of Compound 21

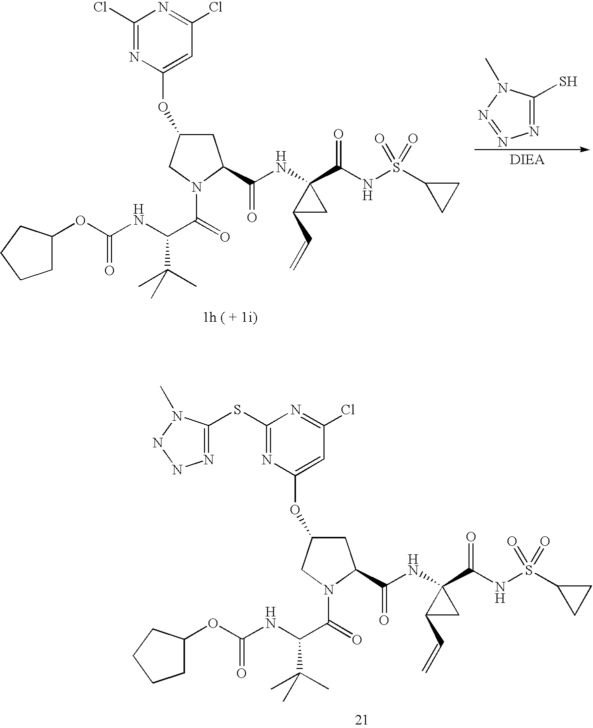

A solution of 50.3 mg (0.070 mmol) of the dichloropyrimidine 1h, 9.1 mg (0.078 mmol) of 5-mercapto-1-methyltetrazole, and 0.027 mL (0.155 mmol) of diisopropylethylamine in DMF (0.7 mL) was stirred at room temperature for 1 h and at 50° C. for 4.5 h. After additional 9.6 mg (0.083 mmol) of the thiol and 0.027 mL (0.155 mmol) of diisopropylethylamine were added, the solution was stirred at 50° C. for 5.5 h and then heated at 100° C. for 20 min using a microwave reactor. The resulting reaction mixture was acidified by adding 0.03 mL (0.389 mmol) of trifluoroacetic acid and purified by preparative HPLC using water (0.05% TFA) and acetonitrile (0.05% TFA) as eluents to give 16.2 mg (29%) of compound 21 after lyophilization. ¹H NMR (300 MHz, CD₃OD): δ 9.17 (s, 1H), 6.86 (s, 1H), 5.68-5.84 (m, 1H), 5.73 (br, 1H), 5.32 (d, 1H, J=17.1 Hz), 5.14 (d, 1H, J=10.2 Hz), 4.94 (br m, 1H), 4.42 (dd, 1H, J=10.2 and 6.9 Hz), 4.31 (d, 1H, J=12.6 Hz), 4.21 (d, 1H, J=4.5 Hz), 4.14 (s, 3H), 4.03 (dd, 1H, J=11.7 and ˜2 Hz), 2.95 (m, 1H), 2.49 (dd, 1H, J=14.0 and 6.8 Hz), 2.18-2.30 (m, 2H), 1.88 (dd, 1H, J=8.1 and 5.7 Hz), 1.54-1.92 (m, 11H), 1.43 (dd, 1H, J=9.8 and 5.3 Hz), 1.21-1.32 (m, 2H), 1.08 (m, 2H), 1.03 (s, 9H). LC/MS=795 (M⁺+1).

### Example 22

### Preparation of Compound 22

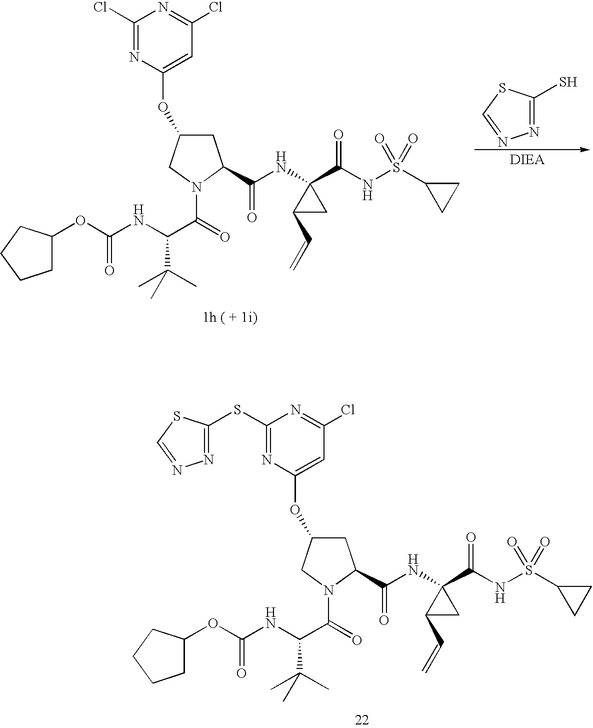

A solution of 50.4 mg (0.070 mmol) of the dichloropyrimidine 1 h, 18.4 mg (0.156 mmol) of 5-mercapto-1-methyltetrazole, and 0.055 mL (0.316 mmol) of diisopropylethylamine in DMF (0.7 mL) was stirred at 100° C. for 20 min using a microwave reactor. The resulting reaction mixture was acidified by adding 0.03 mL (0.389 mmol) of trifluoroacetic acid and purified by preparative HPLC using water (0.05% TFA) and acetonitrile (0.05% TFA) as eluents to give 12.3 mg (29%) of compound 22 after lyophilization. ¹H NMR (300 MHz, CD₃OD): δ 9.66 (s, 1H), 9.18 (s, 1H), 6.96 (s, 1H), 5.68-5.84 (m, 2H), 5.32 (d, 1H, J=17.1 Hz), 5.14 (d, 1H, J=9.9 Hz), 4.33-4.48 (m, 2H), 4.21 (m, 1H), 4.02 (dd, 1H, J=˜12 and ˜2 Hz), 2.95 (m, 1H), 2.51 (dd, 1H, J=13.8 and 6.9 Hz), 2.18-2.32 (m, 2H), 1.89 (dd, 1H, J=7.8 and 5.7 Hz), 1.48-1.86 (m, 9H), 1.43 (dd, 1H, J=9.8 and 5.6 Hz), 1.21-1.32 (m, 2H), 1.07 (m, 2H), 1.03 (s, 9H). LC/MS=797 (M⁺+1) and 819 (M⁺+23).

### Example 23

### Preparation of Compound 23

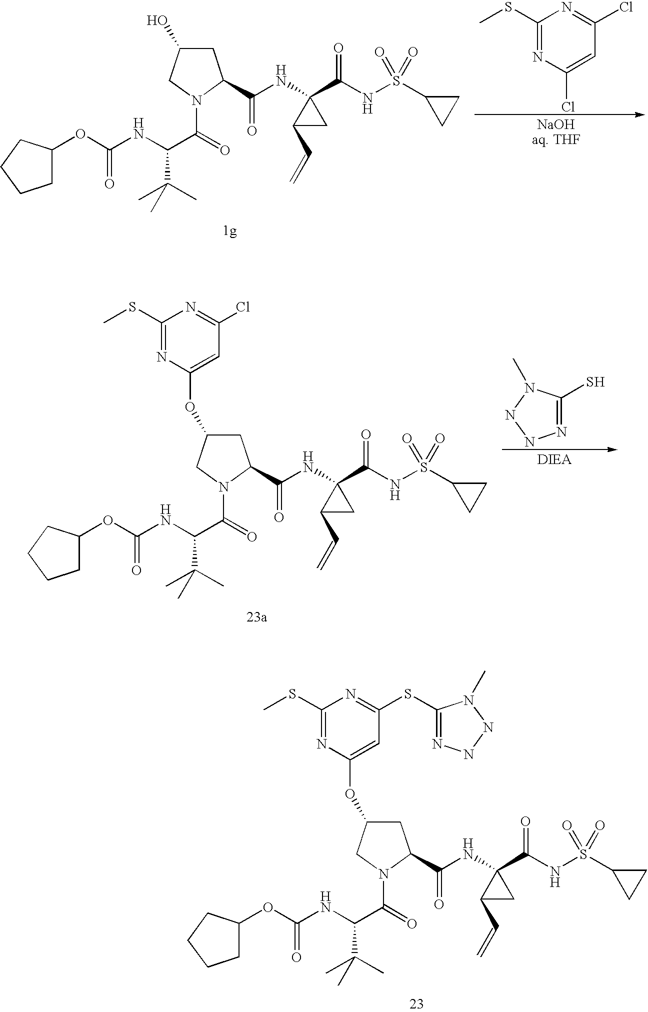

A mixture of 1.50 g (2.64 mmol) of the hydroxyproline 1g and 1.55 g (7.92 mmol) of 4,6-dichloro-2-methylthiopyrimidine in 4 N NaOH (3.3 mL) and THF (9.9 mL) was vigorously stirred at room temperature for 19 h. The mixture was diluted with water (60 mL) and extracted with ethyl acetate (2×50 mL). The extracts were washed with water (×1), combined, dried (MgSO₄), and concentrated. The residue was purified by silica gel column chromatography to obtain 1.32 g (69%) of compound 23a. LC/MS=727 (M⁺+1).

A solution of 50.1 mg (0.069 mmol) of compound 23a, 17.9 mg (0.154 mmol) of 5-mercapto-1-methyltetrazole, and 0.054 mL (0.310 mmol) of diisopropylethylamine in DMF (0.7 mL) was stirred at 100° C. for 10 min and 120° C. for 80 min using a microwave reactor. After the resulting solution was filtered, the filtrate was purified by preparative HPLC using water (0.05% TFA) and acetonitrile (0.05% TFA) as eluents to give 20.4 mg (29%) of compound 23 after lyophilization. ¹H NMR (300 MHz, CD₃OD): δ 6.56 (s, 1H), 5.68-5.88 (m, 2H), 5.32 (d, 1H, J=17.1 Hz), 5.14 (d, 1H, J=9.9 Hz), 4.95 (br, 1H), 4.41 (dd, 1H, J=˜9.3 and ˜7.2 Hz), 4.17-4.28 (m, 2H), 4.11 (s, 3H), 4.03 (dd, 1H, J=˜11 and ˜2 Hz), 2.95 (m, 1H), 2.47 (dd, 1H, J=13.8 and 7.2 Hz), 2.25 (m, 5H), 1.88 (dd, 1H, J=8.3 and 5.6 Hz), 1.54-1.9 (m, 10H), 1.43 (dd, 1H, J=9.5 and 5.3 Hz), 1.21-1.32 (m, 2H), 1.08 (m, 2H), 1.03 (s, 9H). LC/MS=807 (M⁺+1) and 829 (M⁺+23).

### Example 24

### Preparation of Compounds 24a and 24b

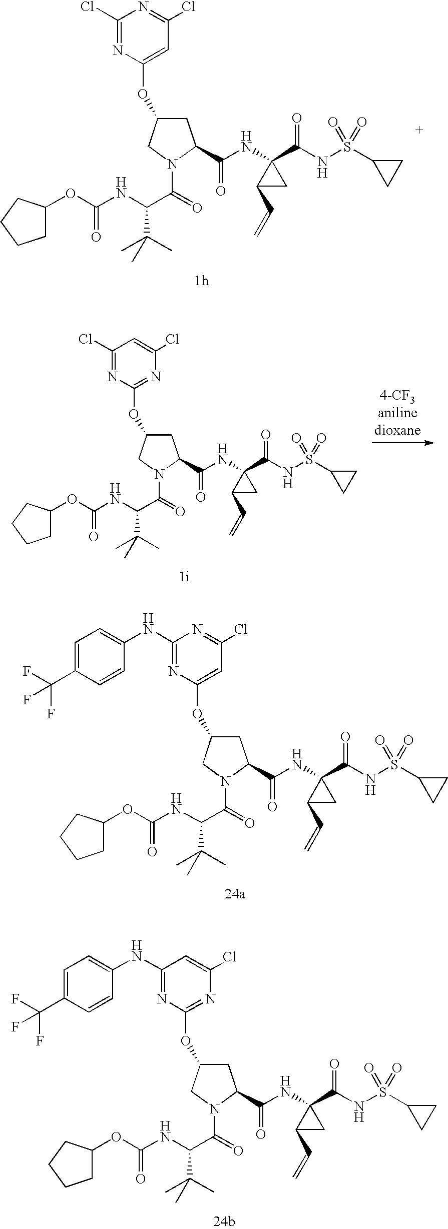

Compounds 24a and 24b were prepared by following procedures similar to those described for compounds 3a and 3b except using 4-trifluoromethylaniline. 24a: MS (m/z) 840 [M+H]⁺, LC/MS Rₜ=2.38 min.

24b: MS (m/z) 840 [M+H]⁺, LC/MS Rₜ=2.31 min.

### Example 25

### Preparation of Compounds 25a and 25b

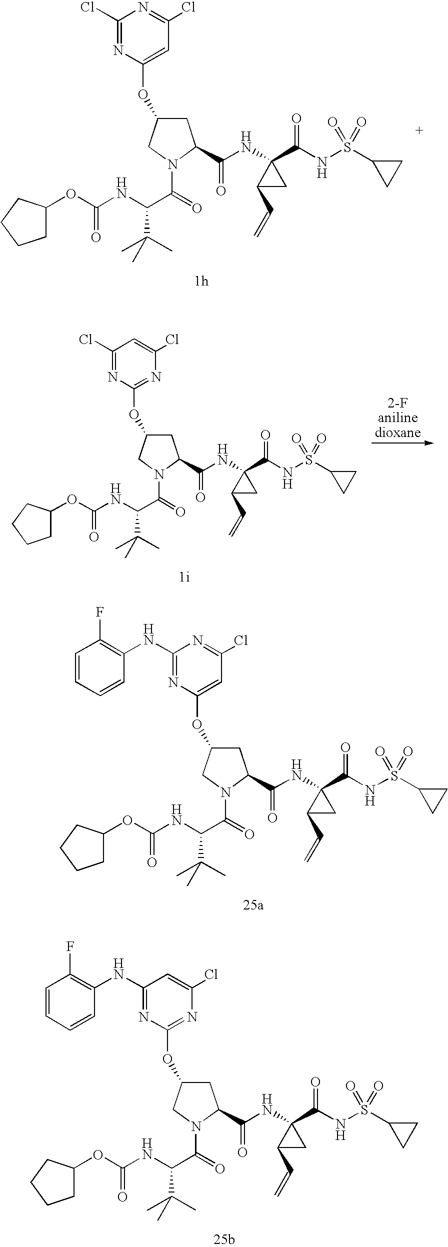

Compounds 25a and 25b were prepared by following procedures similar to those described for compounds 3a and 3b except using 2-fluoroaniline.

25a: MS (m/z) 790 [M+H]⁺, LC/MS Rₜ=2.52 min. 25b: MS (m/z) 790 [M+H]⁺, LC/MS Rₜ=2.44 min.

### Example 26

### Preparation of Compounds 26a and 26b

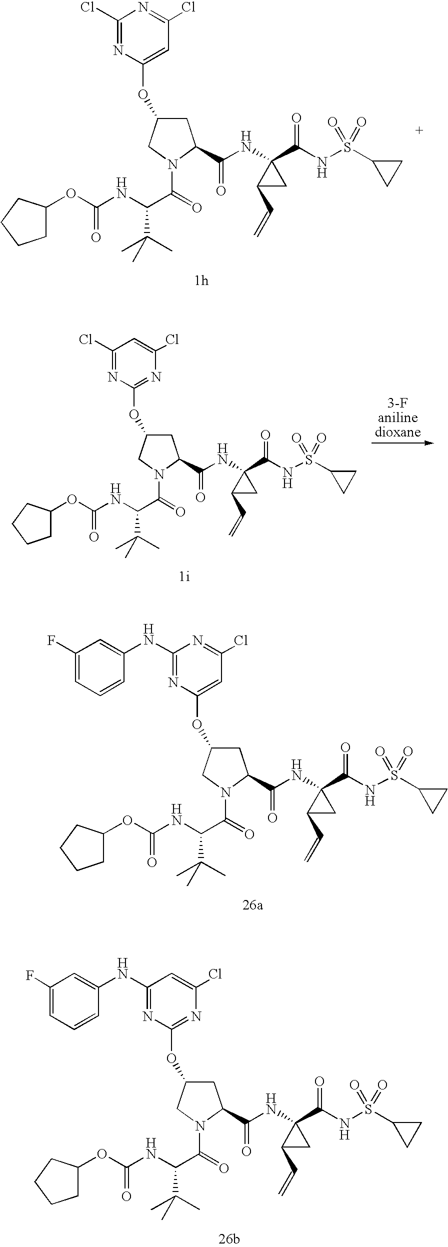

Compounds 26a and 26b were prepared by following procedures similar to those described for compounds 3a and 3b except using 3-fluoroaniline.

26a: MS (m/z) 790 [M+H]⁺, LC/MS Rₜ=2.52 min. 26b: MS (m/z) 790 [M+H]⁺, LC/MS Rₜ=1.94 min.

### Example 27

### Preparation of Compound 27

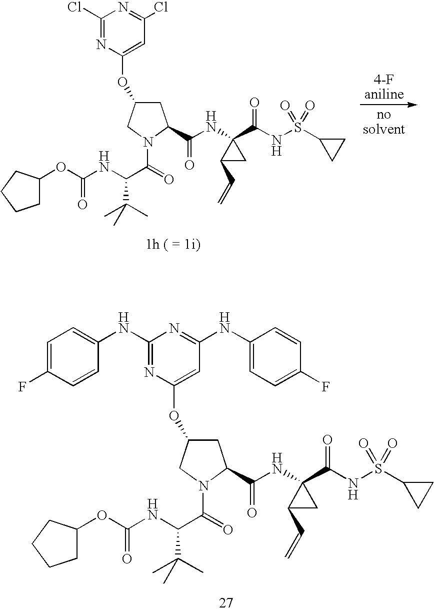

A mixture of 1h (260 mg, 0.36 mmol) and 4-fluoroaniline (0.8 mL) was subjected to a microwave reactor for 20 min at 140° C. The crude was purified by preparative HPLC using water (0.05% TFA) and acetonitrile (0.05% TFA) as eluents to give 51 mg (16%) of 27 as a white solid after lyophilization. MS (m/z) 865.4 [M+H]⁺, LC/MS Rₜ=3.01 min. ¹H NMR (300 MHz, CD₃OD): δ 9.20 (s, 1H), 7.55 (m, 2H), 7.48 (m, 2H), 7.10 (m, 4H), 5.76 (m, 1H), 5.58 (brl, 1H), 5.31 (d, 1H), 5.13 (d, 2H), 4.89 (s, 1H), 4.43 (m, 1H), 4.28 (m, 2H), 4.00 (dd, 1H), 2.95 (m, 1H), 2.47 (m, 1H), 2.24 (m, 2H), 1.4-1.9 (m, 10H), 1.25 (m, 2H), 1.03 (m, 11H).

### Example 28

### Preparation of Compound 28

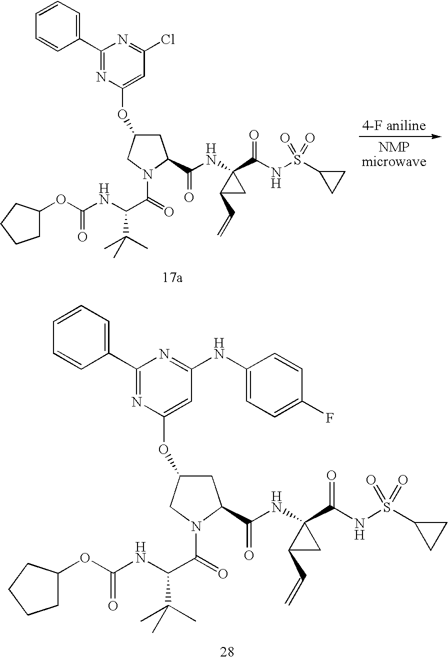

Compound 28 was prepared by following procedures similar to those described for compound 17 except using 4-fluoroaniline.

28: MS (m/z) 832 [M+H]⁺, LC/MS Rₜ=2.93 min.

### Example 29

### Preparation of Compound 29

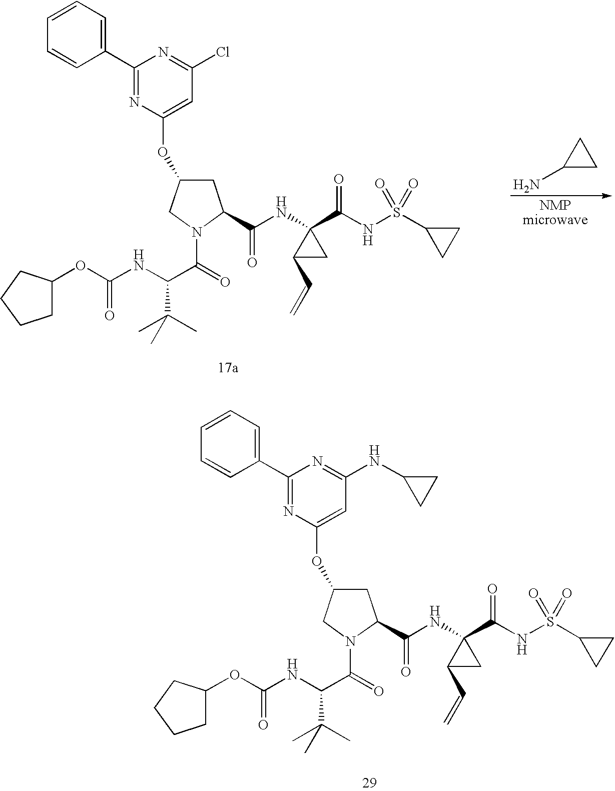

Compound 29 was prepared by following procedures similar to those described for compound 17 except using 4-fluoroaniline. 28: MS (m/z) 778 [M+H]⁺, LC/MS Rₜ=2.29 min.

### Example 30

### Preparation of Compound 30

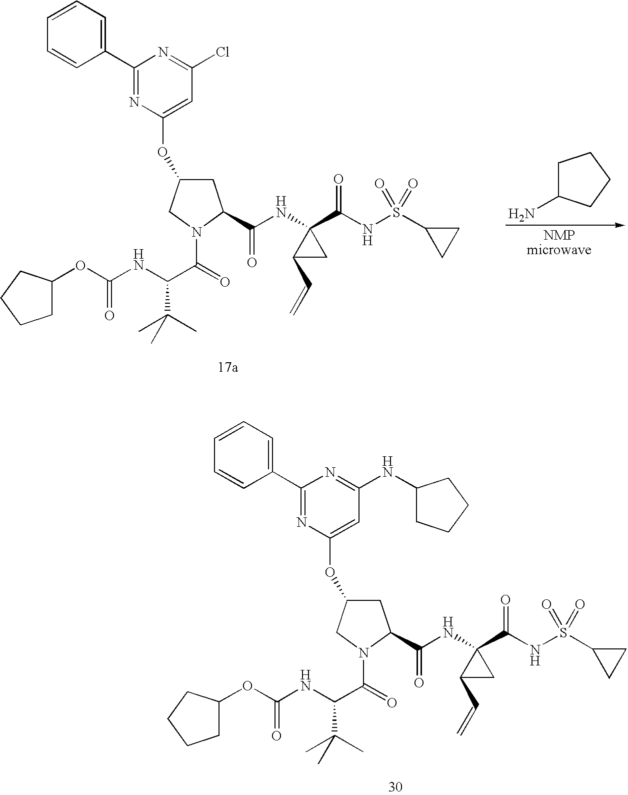

Compound 30 was prepared by following procedures similar to those described for compound 17 except using cyclopentyl amine. 30: MS (m/z) 806.5 [M+H]⁺, LC/MS Rₜ=3.26 min.

### Example 31

### Preparation of Compounds 31a and 31b

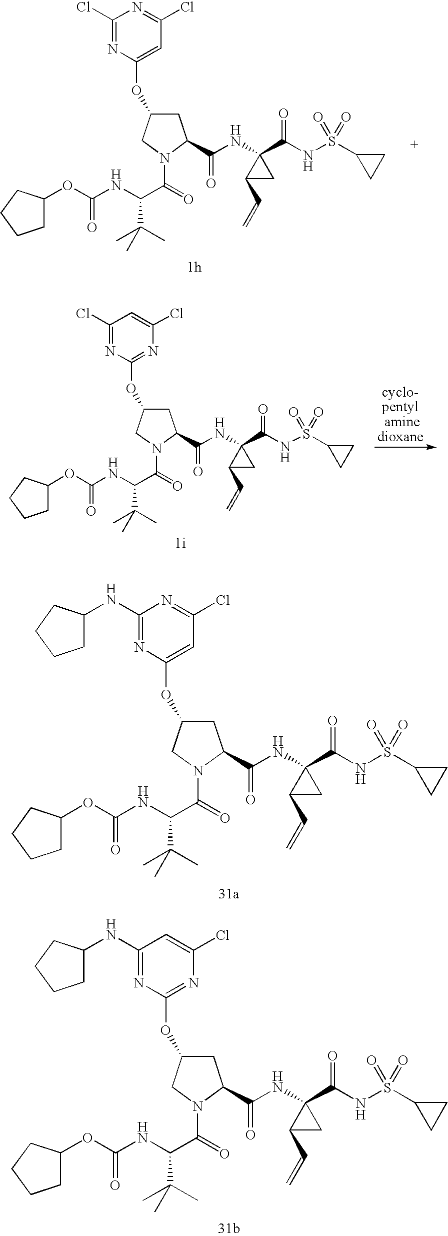

Compounds 31a and 31b were prepared by following procedures similar to those described for compounds 3a and 3b except using cyclopentyl amine. 31a: MS (m/z) 764 [M+H]⁺, LC/MS Rₜ=3.12 min. 31b: MS (m/z) 764 [M+H]⁺, LC/MS Rₜ=3.02 min.

### Example 32

### Preparation of Compound 32

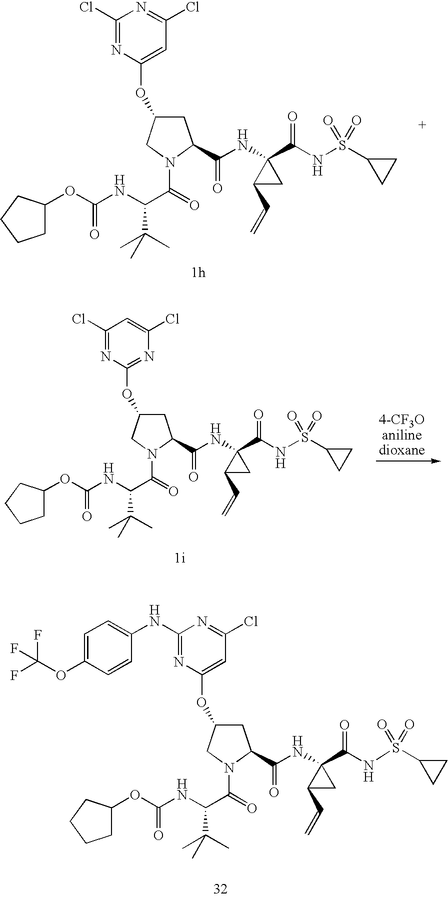

Compound 32 were prepared by following procedures similar to those described for compound 3a except using 4-(trifluoromethoxy)aniline. 32: MS (m/z) 856.4 [M+H]⁺, LC/MS Rₜ=3.21 min.

### Example 33

### Preparation of Compound 33

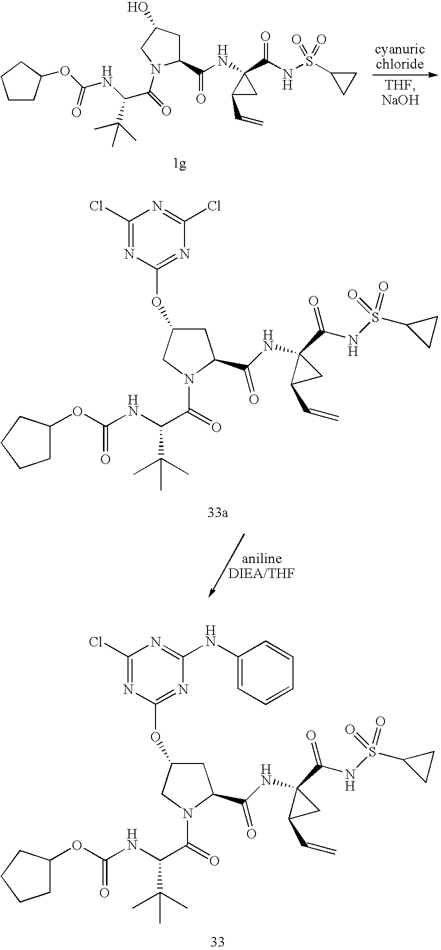

The 4-hydroxyproline 1g (1.0 g, 1.76 mmol) in THF was treated with NaOH (4 N, 1.3 mL, 5.2 mmol) followed by slow addition of cyanuric chloride (1.30 g, 7.1 mmol). After 3 h at room temperature, the mixture was cooled to 0° C. and quenched with HCl (1N, 9 mL). The aqueous layer was extracted with ethylacetate (3×30 mL) and the combined organic layers were washed with brine, dried over magnesium sulfate, and concentrated in vacuo. The crude residue was purified by silica gel chromatography (ethylacetate/hexanes) to afford 608 mg of 33a in 48% yield. ¹H NMR (300 MHz, CD₃OD): δ 6.89 (d, J=9.3 Hz, 1H), 5.82-5.68 (m, 2H), 5.32 (d, J=16.8 Hz, 1H), 5.15 (d, J=11.4 Hz, 1H), 4.55-4.45 (m, 2H), 4.18 (d, J=9.6 Hz, 1H), 4.03 (bd, J=9.3 Hz, 1H), 2.99-2.81 (m, 1H), 2.57 (dd, J=14.1, 7.2 Hz, 1H), 2.34-2.20 (m, 2H), 1.89 (dd, J=8.1, 5.4 Hz, 1H), 1.87-1.48 (m, 8H), 1.44 (dd, J=9.6, 5.4 Hz, 1H), 1.29-1.23 (m, 2H), 1.11-1.02 (m, 2H), 1.02 (s, 9H); LCMS (M+1): 717.86.

A solution of the dichlorotriazine 33a (82 mg, 0.11 mmol), diisopropylethylamine (0.04 mL, 0.23 mmol), and aniline (0.011 mL, 0.12 mmol) in THF (1.1 mL) was heated to 60° C. for 1.25 h. Upon cooling to room temperature, HCl (1N, 230 μL) was added along with ethyl acetate (10 mL). The organic layer was washed with saturated ammonium chloride solution (10 mL), brine (5 mL), dried over magnesium sulfate, and concentrated in vacuo. The crude residue was purified by silica gel chromatography (ethyl acetate/hexanes) to afford 64 mg of 33 in 73% yield. ¹H NMR (300 MHz, CD₃OD): δ 7.63 (d, J=8.1 Hz, 2H), 7.39-7.33 (m, 2H), 7.18-7.12 (m, 1H), 6.93-6.90 (m, 1H), 5.83-5.61 (m, 2H), 5.32 (d, J=17.1 Hz, 1H), 5.14 (d, J=10.2 Hz, 1H), 4.92-4.84 (m, 1H), 4.50-4.39 (m, 2H), 4.25 (bs, 1H), 4.02-3.96 (m, 1H), 2.98-2.91 (m, 1H), 2.56-2.49 (m, 1H), 2.30-2.23 (m, 2H), 1.92-1.85 (m, 1H), 1.85-1.55 (m, 8H), 1.55-1.41 (m, 1H), 1.30-1.20 (m, 2H), 1.10-1.00 (m, 2H), 1.01 (s, 9H); LCMS (M+1): 772.91.

### Example 34

### Preparation of Compound 34

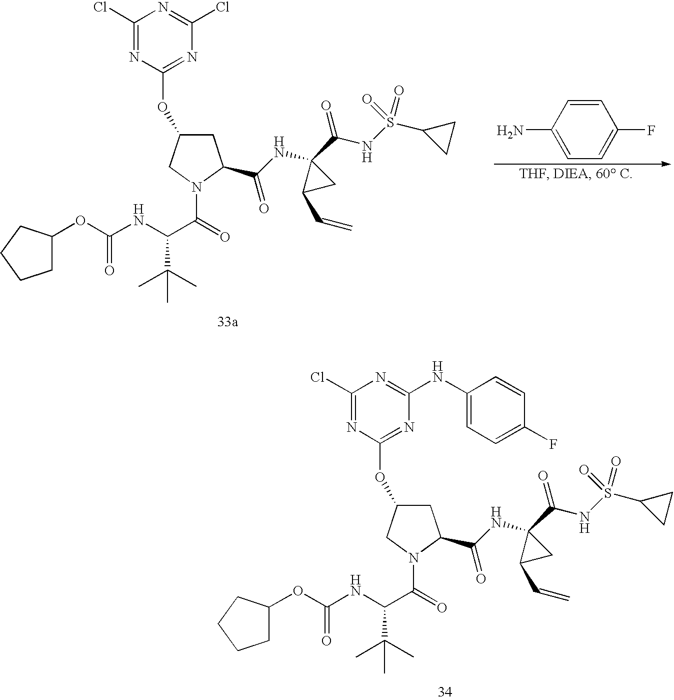

Compound 34 was obtained by following procedures similar to those described for compound 33 except using 4-fluoroaniline. 34: MS (m/z) 790.95 [M+H]⁺.

### Example 35

### Preparation of Compound 35

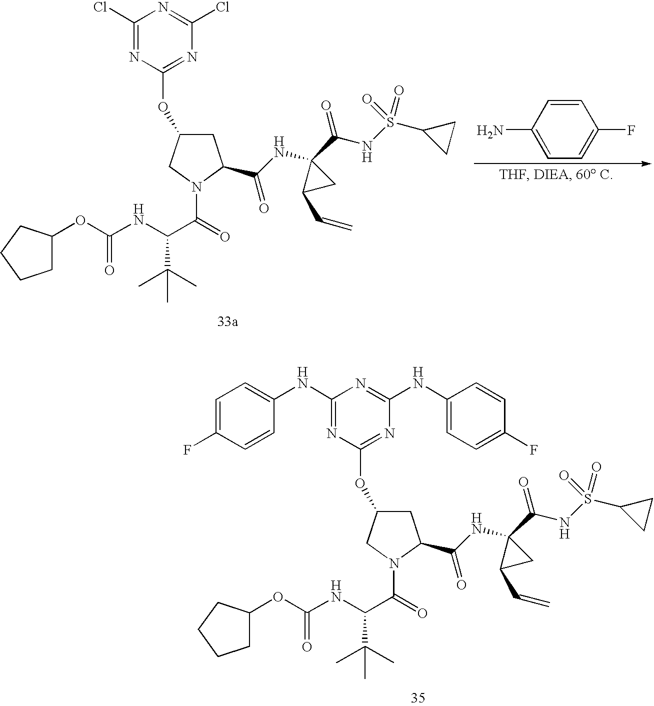

A solution of the dichlorotriazine 33a (64 mg, 0.090 mmol), 4-fluoroaniline (0.051 mL, 0.54 mmol), diisopropylethylamine (0.095 mL, 0.55 mmol) in THF (0.9 mL) was stirred at reflux for 10 h. Upon cooling to room temperature, HCl (1N, 230 μL) was added along with ethyl acetate (10 mL). The organic layer was washed with saturated ammonium chloride solution (10 mL), brine (5 mL), dried over magnesium sulfate, and concentrated in vacuo, affording 78 mg of 35 in quantitative yield. ¹H NMR (300 MHz, CD₃OD): δ 7.72-7.53 (m, 4H), 7.15-7.00 (m, 4H), 7.00-6.91 (m, 1H), 5.83-5.71 (m, 1H), 5.71-5.64 (m, 1H), 5.31 (d, J=12.9 Hz, 1H), 5.13 (d, J=7.8 Hz, 1H), 4.52-4.38 (m, 1H), 4.37-4.20 (m, 2H), 4.05-3.96 (m, 1H), 2.98-2.91 (m, 1H), 2.53-2.46 (m, 1H), 2.30-2.26 (m, 2H), 1.92-1.87 (m, 1H), 1.83-1.41 (m, 8H), 1.40-1.34 (m, 1H), 1.30-1.22 (m, 2H), 1.12-1.05 (m, 2H), 1.01 (s, 9H); LCMS (M+1): 866.09.

### Example 36

### Preparation of Compound 36

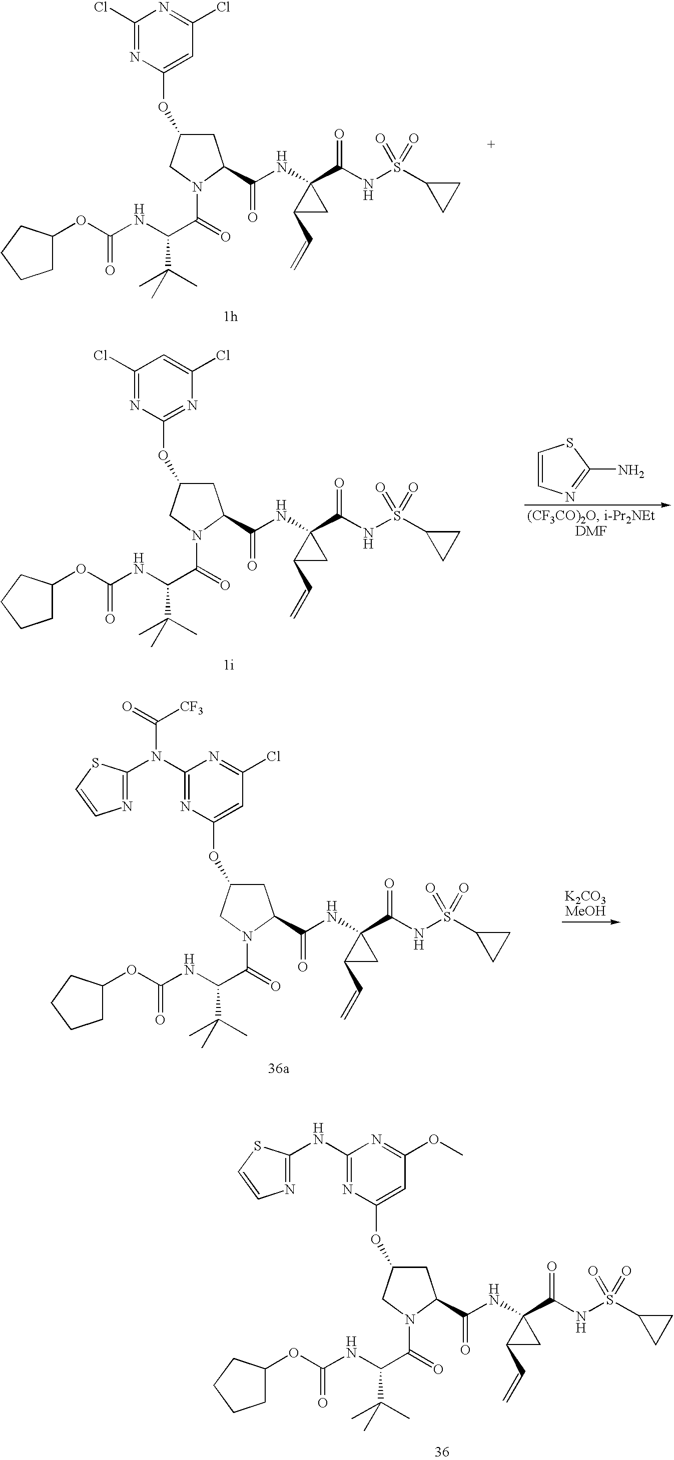

To a solution of 2-aminothiazole (105 mg, 1.05 mmol) in DMF (1 mL) were added trifluoroactic anhydride (0.1 mL, 0.72 mmol) and diisopropylethylamine (0.25 mL, 1.43 mmol) at 0° C. After the solution was stirred at 0° C. for 2 h and at room temperature for 1 h, a solution of a mixture of 1h and 1i (50 mg, 0.07 mmol) in DMF (0.6 mL) was added to the reaction mixture and the resulting solution was heated at 100° C. for 50 min using a microwave reactor. The solution was acidified with trifluoroacetic acid (0.12 mL, 1.56 mmol) and purified by preparative HPLC using water (0.05% TFA) and acetonitrile (0.05% TFA) as eluents to give 10.4 mg (17%) of 36a as a white solid after lyophilization. MS (m/z) 875.0 [M+H]⁺ and 897.0 [M+Na]⁺, LC/MS Rₜ=4.77 min.

A mixture of compound 36a (10.4 mg, 0.012) and potassium carbonate (7.9 mg, 0.057 mmol) in methanol was stirred at room temperature for 18.5 h. After the solution was acidified by adding a drop of trifluoroacetic acid, the solution was concentrated and the residue was dissolved in DMF and purified by preparative HPLC using water (0.05% TFA) and acetonitrile (0.05% TFA) as eluents to give 1.6 mg (17%) of 36 as a white solid after lyophilization. MS (m/z) 875.0 [M+H]⁺ and 897.0 [M+Na]⁺, LC/MS Rₜ=4.77 min. ¹H NMR (300 MHz, CD₃OD): δ 9.23 (s, 1H), 7.84 (d, 1H, J=4.8 Hz), 7.14 (d, 1H, J=4.8 Hz), 6.95 (s, 1H), 5.69-5.84 (m, 2H), 5.32 (d, 1H, J=17.4 Hz), 5.15 (d, 1H, J=9.9 Hz), 4.8˜4.96 (br, 1H), 4.46-4.56 (m, 1H), 4.37-4.46 (m, 1H), 4.25 (m, 1H), 4.11 (s, 3H), 2.96 (m, 1H), 2.57 (dd, 1H, J=˜15 and ˜6 Hz), 2.19-2.38 (m, 2H), 1.90 (dd, 1H, J=7.8 and 5.7 Hz), 1.49-1.85 (m, 9H), 1.44 (dd, 1H, J=9.5 and 5.3 Hz), 1.2-1.35 (m, 2H), 1.09 (m, 2H), 1.04 (s, 9H). LC/MS=775.3 [M+H]⁺, LC/MS Rₜ=4.98 min.

### Example 37

### Preparation of Compound 37

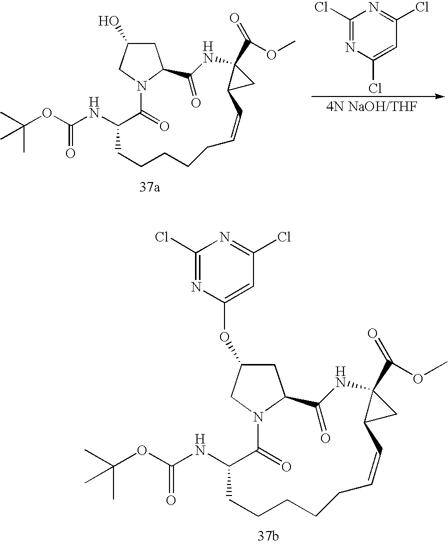

To a solution of compound 37a (960 mg, 2.0 mmol, obtained according to J. Org. Chem., 70, 2005, 10765, starting with trans-N-(tert-butoxycarbonyl)-4-hydroxy-L-proline) and 2,4,6-trichloropyrimidine (734 mg, 4.0 mmol) in THF (9 mL) was added 4N sodium hydroxide (3 mL, 1.2 mmol) and the resulting mixture was stirred at room temperature for 2 h. The mixture was partitioned between ethyl acetate (80 mL) and brine (80 mL). The organic layer was washed with brine, dried over sodium sulfate, and concentrated in vacuo. The residue was purified by silica gel column chromatography (ethyl acetate/hexanes), affording compound 37b (1.2 g, 96%) as a white solid. MS=648.1 (M⁺+Na).

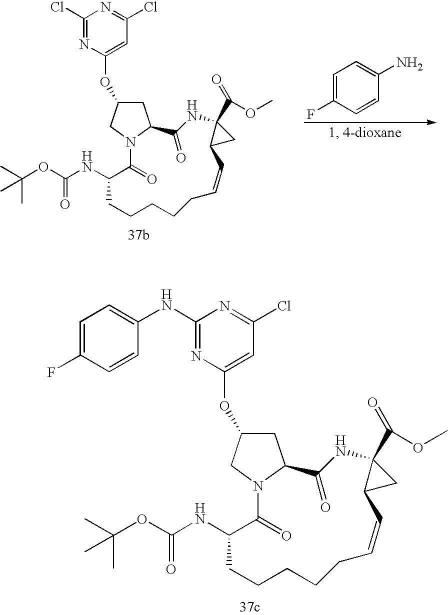

A solution of the ester 37b (1.10 g, 1.76 mmol) and 4-fluoroaniline (1.67 mL, 17.6 mmol) in 1,4-dioxane (10 mL) was stirred at 120° C. for 6 h. The reaction was nearly complete. The mixture was concentrated to remove the organic solvent and re-dissolved in ethyl acetate (100 mL). The solution was washed with a mixed aqueous solution of 1N HCl (50 mL) and brine (50 mL) and then with brine, dried over sodium sulfate, and concentrated in vacuo. The residue was purified by silica gel column chromatography (ethyl acetate/hexanes), affording compound 37c (0.62 g, 50%) as a white solid. MS=701.4 (M⁺+H).

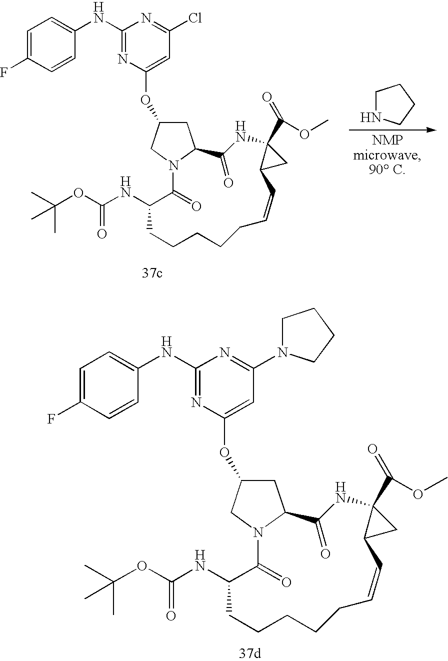

A solution of the ester 37c (620 mg, 0.88 mmol) and pyrrolidine (2 mL) in NMP (5 mL) was subjected to a microwave reactor at 90° C. for 10 min. The mixture was partitioned between ethyl acetate (50 mL) and 3% aqueous LiCl (50 mL). The organic layer was washed sequentially with 3% aqueous LiCl (50 mL), 1N HCl (30 mL) and brine, and then concentrated in vacuo. The crude compound 37d was used for the next reaction. MS=736.5 (M⁺+H).

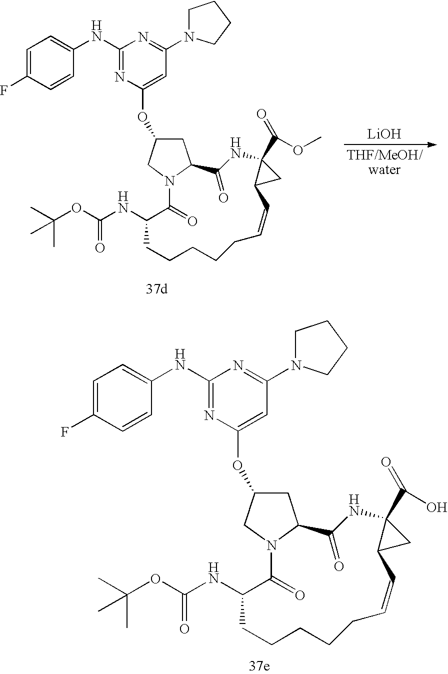

To a solution of compound 37d obtained from the previous step in THF (6 mL) and MeOH (4 mL) was added a solution of lithium hydroxide hydrate (470 mg) in water (2 mL) and stirred at room temperature for 4 h. The reaction was complete. The mixture was partitioned between ethyl acetate (50 mL) and brine (50 mL). The aqueous layer was neutralized to ˜pH 5 with 4 N HCl. The organic layer was taken. The aqueous layer was extracted with ethyl acetate (30 mL). The combined organic layers were washed with brine, dried over sodium sulfate, concentrated in vacuo, affording compound 37e (600 mg, 94% in two steps) as a white solid. MS=722.5 (M⁺+H).

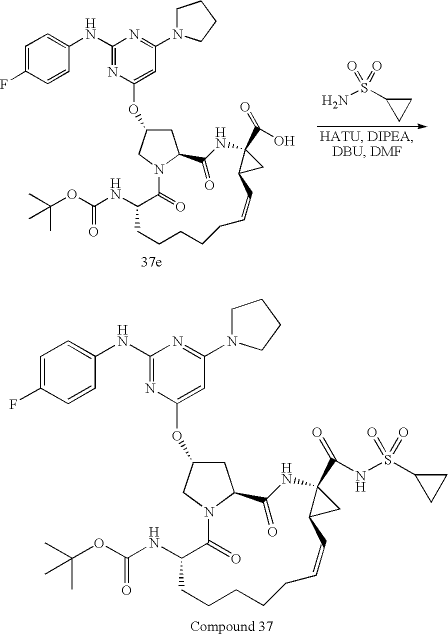

To a solution of compound 37e (400 mg, 0.55 mmol) in DMF (3 mL) were added HATU (320 mg, 0.84 mmol) and DIPEA (0.15 mL, 0.84 mmol) and stirred for 1 h. Cyclopropylsulfonamide (136 mg, 1.12 mmol) and DBU (0.34 mL, 2.24 mmol) were added. The resulting mixture was stirred at room temperature for 16 h. The mixture was partitioned between ethyl acetate (100 mL) and saturated sodium bicarbonate (100 mL). The organic layer was washed with 1N HCl/brine (100 mL, 1/1), dried over sodium sulfate, and concentrated in vacuo. The residue was purified by RP-HPLC (acetonitrile/water, 0.05% TFA), affording compound 37 (325 mg, 72%) as a white solid. ¹H NMR (300 MHz, CD₃OD): δ 9.07 (brs, 1H), 7.60 (m, 2H), 7.14 (t, J=8.1 Hz, 2H), 5.69 (dd, J=17.4, 8.4 Hz, 1H), 5.56 (s, 1H), 5.09 (dd, J=8.7, 9.3 Hz, 1H), 4.62 (m, 2H), 4.12 (d, J=9.9 Hz, 1H), 3.92 (dd, J=9.6, 1.2 Hz, 1H), 3.58 (brs, 4H), 2.92 (m, 1H), 2.62 (m, 2H), 2.49 (m, 1H), 2.34 (dd, J=8.4, 17.1 Hz, 1H), 2.08 (brs, 4H), 1.6-1.9 (m, 4H), 1.3-1.6 (m, 8H), 1.27 (s, 9H), 1.10 (m, 2H), 1.02 (m, 1H). MS=825.4 (M⁺+H).

### Example 38

### Preparation of Compound 38

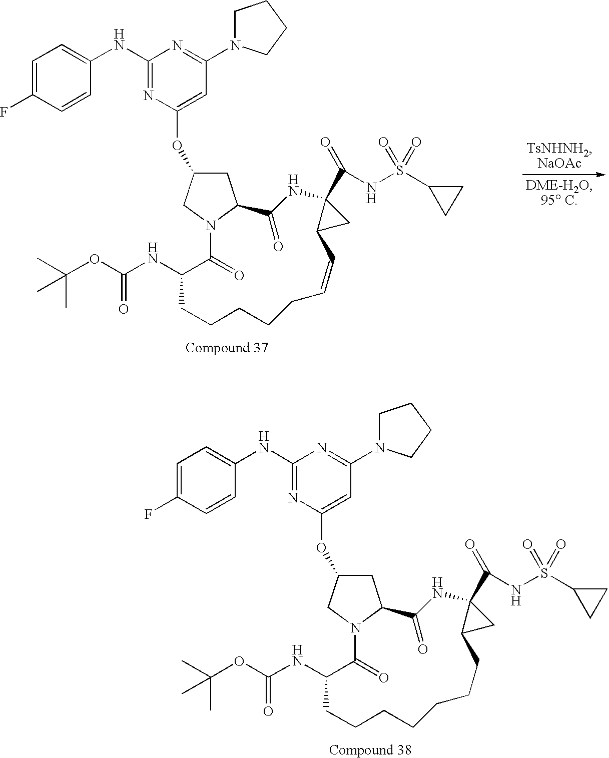

A mixture of compound 37 (120 mg, 0.145 mmol), p-toluenesulfonyl hydrazide (203 mg, 1.1 mmol) and sodium acetate (178 mg, 2.18 mmol) in DME (1.8 mL) and water (0.2 mL) was stirred at 95° C. for 2 h. The mixture was partitioned between ethyl acetate (75 mL) and saturated sodium bicarbonate (75 mL). The organic layer was washed with 0.5N HCl/brine (75 mL, 1/1) and then brine, and concentrated in vacuo. The residue was purified by RP-HPLC (acetonitrile/water, 0.05% TFA), affording compound 38 (89 mg, 74%) as a white solid. ¹H NMR (300 MHz, CD₃OD): δ 9.11 (brs, 1H), 7.59 (m, 2H), 7.15 (t, J=8.1 Hz, 2H), 5.56 (s, 1H), 4.61 (t, J=8.1 Hz, 1H), 4.50 (d, J=12.3 Hz, 1H), 4.22 (d, J=9.9 Hz, 1H), 3.97 (dd, J=9.6, 1.2 Hz, 1H), 3.58 (brs, 4H), 2.98 (m, 1H), 2.61 (m, 1H), 2.37 (m, 1H), 2.06 (brs, 4H), 1.9-1.9 (m, 21H, overlapped with s, 9H at 1.34). MS=827.6 (M⁺+H).

### Biological Assays

NS3 Enzymatic Potency: Purified NS3 protease is complexed with NS4A peptide and then incubated with serial dilutions of compound (DMSO used as solvent). Reactions are started by addition of dual-labeled peptide substrate and the resulting kinetic increase in fluorescence is measured. Non-linear regression of velocity data is performed to calculate IC₅₀s. Activity is initially tested against genotype 1b protease. Depending on the potency obtained against genotype 1b, additional genotypes (1a, 2a, 3) and or protease inhibitor resistant enzymes (D168Y, D168V, or A156T mutants) may be tested. BILN-2061 is used as a control during all assays. Representative compounds of the invention were evaluated in this assay and were typically found to have IC₅₀ values of less than about 1 μm.

Replicon Potency and Cytotoxicity: Huh-luc cells (stably replicating Bartenschlager's 1389luc-ubi-neo/NS3-3′/ET genotype 1b replicon) are treated with serial dilutions of compound (DMSO is used as solvent) for 72 hours. Replicon copy number is measured by bioluminescence and non-linear regression is performed to calculate EC₅₀s. Parallel plates treated with the same drug dilutions are assayed for cytotoxicity using the Promega CellTiter-Glo cell viability assay. Depending on the potency achieved against the 1b replicon, compounds may be tested against a genotype 1a replicon and/or inhibitor resistant replicons encoding D168Y or A156T mutations. BILN-2061 is used as a control during all assays. Representative compounds of the invention were evaluated in this assay and were typically found to have EC₅₀ values of less than about 5 μm.

### Effect of Serum Proteins on Replicon Potency

Replicon assays are conducted in normal cell culture medium (DMEM+110% FBS) supplemented with physiologic concentrations of human serum albumin (40 mg/mL) or a-acid glycoprotein (1 mg/mL). EC₅₀s in the presence of human serum proteins are compared to the EC₅₀ in normal medium to determine the fold shift in potency.

Enzymatic Selectivity The inhibition of mammalian proteases including Porcine Pancreatic Elastase, Human Leukocyte Elastase, Protease 3, and Cathepsin D are measured at Kₘ for the respective substrates for each enzyme. IC₅₀ for each enzyme is compared to the IC₅₀ obtained with NS3 1b protease to calculate selectivity. Representative compounds of the invention have shown activity.

MT-4 Cell Cytotoxicity: MT4 cells are treated with serial dilutions of compounds for a five day period. Cell viability is measured at the end of the treatment period using the Promega CellTiter-Glo assay and non-linear regression is performed to calculate CC₅₀.

Compound Concentration Associated with Cells at EC₅. Huh-luc cultures are incubated with compound at concentrations equal to EC₅₀. At multiple time points (0-72 hours), cells are washed 2× with cold medium and extracted with 85% acetonitrile; a sample of the media at each time-point will also be extracted. Cell and media extracts are analyzed by LC/MS/MS to determine the Molar concentration of compounds in each fraction. Representative compounds of the invention have shown activity.

Solubility and Stability: Solubility is determined by taking an aliquot of 10 mM DMSO stock solution and preparing the compound at a final concentration of 100 μM in the test media solutions (PBS, pH 7.4 and 0.1 N HCl, pH 1.5) with a total DMSO concentration of 1%. The test media solutions are incubated at room temperature with shaking for 1 hr. The solutions will then be centrifuged and the recovered supernatants are assayed on the HPLC/UV. Solubility will be calculated by comparing the amount of compound detected in the defined test solution compared to the amount detected in DMSO at the same concentration. Stability of compounds after an 1 hour incubation with PBS at 37° C. will also be determined.

Stability in Cryopreserved Human Dog, and Rat Hepatocytes: Each compound is incubated for up to 1 hour in hepatocyte suspensions (100 μl, 80,000 cells per well) at 37° C. Cryopreserved hepatocytes are reconstituted in the serum-free incubation medium. The suspension is transferred into 96-well plates (50 μL/well). The compounds are diluted to 2 μM in incubation medium and then are added to hepatocyte suspensions to start the incubation. Samples are taken at 0, 10, 30 and 60 minutes after the start of incubation and reaction will be quenched with a mixture consisting of 0.3% formic acid in 90% acetonitrile/10% water. The concentration of the compound in each sample is analyzed using LC/MS/MS. The disappearance half-life of the compound in hepatocyte suspension is determined by fitting the concentration-time data with a monophasic exponential equation. The data will also be scaled up to represent intrinsic hepatic clearance and/or total hepatic clearance.

Stability in Hepatic S9 Fraction from Human Dog, and Rat: Each compound is incubated for up to 1 hour in S9 suspension (500 μl, 3 mg protein/mL) at 37° C. (n=3). The compounds are added to the S9 suspension to start the incubation. Samples are taken at 0, 10, 30, and 60 minutes after the start of incubation. The concentration of the compound in each sample is analyzed using LC/MS/MS. The disappearance half-life of the compound in S9 suspension is determined by fitting the concentration-time data with a monophasic exponential equation.

Caco-2 Permeability: Compounds are assayed via a contract service (Absorption Systems, Exton, Pa.). Compounds are provided to the contractor in a blinded manner. Both forward (A-to-B) and reverse (B-to-A) permeability will be measured. Caco-2 monolayers are grown to confluence on collagen-coated, microporous, polycarbonate membranes in 12-well Costar Transwell® plates. The compounds are dosed on the apical side for forward permeability (A-to-B), and are dosed on the basolateral side for reverse permeability (B-to-A). The cells are incubated at 37° C. with 5% CO2 in a humidified incubator. At the beginning of incubation and at 1 hr and 2 hr after incubation, a 200-μL aliquot is taken from the receiver chamber and replaced with fresh assay buffer. The concentration of the compound in each sample is determined with LC/MS/MS. The apparent permeability, Papp, is calculated.

### Plasma Protein Binding:

Plasma protein binding is measured by equilibrium dialysis. Each compound is spiked into blank plasma at a final concentration of 2 μM. The spiked plasma and phosphate buffer is placed into opposite sides of the assembled dialysis cells, which will then be rotated slowly in a 37° C. water bath. At the end of the incubation, the concentration of the compound in plasma and phosphate buffer is determined. The percent unbound is calculated using the following equation:

$$\% Unbound = 100 \cdot ( \frac{{C}_{f}}{{C}_{b} + {C}_{f}} )$$

Where C_{f} and C_{b} are free and bound concentrations determined as the post-dialysis buffer and plasma concentrations, respectively.

### CYP450 Profiling:

Each compound is incubated with each of 5 recombinant human CYP450 enzymes, including CYP1A2, CYP2C9, CYP3A4, CYP2D6 and CYP2C19 in the presence and absence of NADPH. Serial samples will be taken from the incubation mixture at the beginning of the incubation and at 5, 15, 30, 45 and 60 min after the start of the incubation. The concentration of the compound in the incubation mixture is determined by LC/MS/MS. The percentage of the compound remaining after incubation at each time point is calculated by comparing with the sampling at the start of incubation.

### Stability in Rat, Dog, Monkey and Human Plasma:

Compounds will be incubated for up to 2 hour in plasma (rat, dog, monkey, or human) at 37° C. Compounds are added to the plasma at final concentrations of 1 and 10 ug/mL. Aliquots are taken at 0, 5, 15, 30, 60, and 120 min after adding the compound. Concentration of compounds and major metabolites at each timepoint are measured by LC/MS/MS.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed:

1. A compound of formula I: 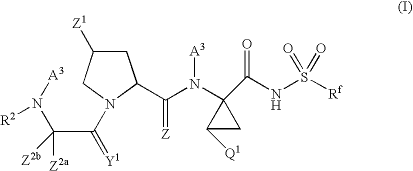
or a pharmaceutically acceptable salt, or prodrug thereof, wherein:
R¹ is independently selected from H, alkyl, alkenyl, alkynyl, aryl, cycloalkyl, heterocycle, halogen, haloalkyl, alkylsulfonamido, arylsulfonamido, —C(O)NHS(O)₂—, or —S(O)₂—, optionally substituted with one or more A³;
R² is selected from,
a) —C(Y¹)(A³),
b) (C2-10)alkyl, (C3-7)cycloalkyl or (C1-4)alkyl-(C3-7)cycloalkyl, where said cycloalkyl and alkyl-cycloalkyl may be optionally mono-, di- or tri-substituted with (C1-3)alkyl, or where said alkyl, cycloalkyl and alkyl-cycloalkyl may optionally be mono- or di-substituted with substituents selected from hydroxy and O—(C1-4)alkyl, or
where each of said alkyl groups may optionally be mono-, di- or tri-substituted with halogen, or
where each of said cycloalkyl groups being 5-, 6- or 7-membered, one or two —CH₂— groups not being directly linked to each other may be optionally replaced by —O— such that the O-atom is linked to the N atom to which R² is attached via at least two C-atoms,
c) phenyl, (C1-3)alkyl-phenyl, heteroaryl or (C1-3)alkyl-heteroaryl,
wherein the heteroaryl groups are 5- or 6-membered having from 1 to 3 heteroatoms selected from N, O and S, wherein said phenyl and heteroaryl groups may optionally be mono-, di- or trisubstituted with substituents selected from halogen, —OH, (C1-4)alkyl, O—(C1-4)alkyl, S—(C1-4)alkyl, —NH₂, —CF₃, —NH((C1-4)alkyl) and —N((C1-4)alkyl)₂, —CONH₂ and —CONH—(C1-4)alkyl; and wherein said (C1-3)alkyl may optionally be substituted with one or more halogen;
d) —S(O)₂(A³); or
e) —C(Y¹)—X—Y;
each R³ is independently H or (C1-6)alkyl;
Y¹ is independently O, S, N(A3), N(O)(A³), N(OA³), N(O)(OA³) or N(N(A³)(A³));
Z is O, S, or NR³;
each R_{c} is R⁴, H, cyano, F, Cl, Br, I, —C(═O)NR_{d}Rₑ, C(═O)NRₛRₜ, NRₛRₜ, S(═O)₂NRₛRₜ, (C1-10)alkoxy, cycloalkyl, aryl, or heteroaryl, which aryl or heteroaryl is optionally substituted with one or more groups independently selected from halo, hydroxy, (C1-10)alkyl, (C2-10)alkenyl, (C2-10)alkynyl, (C1-10)alkanoyl, (C1-10)alkoxy, (C1-10)alkanoyloxy, (C1-10)alkoxycarbonyl, NRₙRₚ, SRᵣ, S(O)Rᵣ, or S(O)₂Rᵣ;
R_{d} and Rₑ are each independently H or (C1-10)alkyl;
Z^{2b} is H, (C1-10)alkyl, (C2-10)alkenyl, or (C2-10)alkynyl;
Q¹ is (C1-10)alkyl, (C2-10)alkenyl, or (C2-10)alkynyl which Q¹ is optionally substituted with R⁴ or R_{c}; or Q¹ and Z^{2a} taken together with the atoms to which they are attached form a heterocycle, which heterocycle may optionally be substituted with one or more oxo (═O), R⁴, or A³;
each X is independently a bond, O, S, or NR³;
Y is a polycarbocycle or a polyheterocycle, which polycarbocycle or a polyheterocycle is optionally substituted with one or more R⁴, halo, carboxy, hydroxy, (C1-10)alkyl, (C2-10)alkenyl, (C2-10)alkynyl, (C1-10)alkanoyl, (C1-10)alkoxy, (C1-10)alkanoyloxy, (C1-10)alkoxycarbonyl, NRₙRₚ, SRᵣ, S(O)Rᵣ, or S(O)₂Rᵣ;
each R⁴ is independently —P(Y³)(OA²)(OA²), —P(Y³)(OA²)(N(A²)₂), —P(Y³)(A²)(OA²), —P(Y³)(A²)(N(A²)₂), or P(Y³)(N(A²)₂)(N(A²)₂);
each Y³ is independently O, S, or NR³;
each Rₙ and Rₚ is independently H, (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, which (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, is optionally substituted with one or more R¹, halo, hydroxy, carboxy, cyano, or (C1-10)alkoxy; or Rₙ and Rₚ together with the nitrogen to which they are attached form a pyrrolidine, piperidine, piperazine, morpholino, or thiomorpholino ring;
each Rᵣ is independently H, (C1-10)alkyl, (C2-10)alkenyl, (C2-10)alkynyl, (C1-10)alkanoyl, heterocycle, or (C1-10)alkoxycarbonyl, wherein any (C1-10)alkyl, (C2-10)alkenyl, (C2-10)alkynyl, (C1-10)alkanoyl, heterocycle, or (C1-10)alkoxycarbonyl is optionally substituted with one or more A³;
each Rₛ and Rₜ is independently H, (C1-10)alkyl, (C2-10)alkenyl, (C2-10)alkynyl, (C1-10)alkanoyl, S(═O)₂A², (C1-10)alkoxy, (C1-10)alkanoyloxy, or (C1-10)alkoxycarbonyl, which (C1-10)alkyl, (C2-10)alkenyl, (C2-10)alkynyl, (C1-10)alkanoyl, (C1-10)alkoxy, (C1-10)alkanoyloxy, or (C1-10)alkoxycarbonyl, is optionally substituted with one or more R¹, halo, hydroxy, carboxy, cyano, or (C1-10)alkoxy; or Rₛ and Rₜ together with the nitrogen to which they are attached form a pyrrolidine, piperidine, piperazine, morpholino, or thiomorpholino ring wherein one or more carbon atoms of said pyrrolidine, piperidine, piperazine, morpholino or thiomorpholino ring is optionally replaced by S(═O), S(═O)₂, or C(═O);
Z^{2a} is H, (C1-10)alkyl, (C2-10)alkenyl, (C2-10)alkynyl, haloalkyl, (C1-10)alkyl-S(═O)₂—(C1-10)alkyl, or cycloalkyl, wherein any carbon atom of Z^{2a} may optionally be replaced with a heteroatom selected from O, S or N and wherein any cycloalkyl is optionally substituted with one or more (C1-10)alkyl, (C2-10)alkenyl, (C2-10)alkynyl, F, Cl, Br, or I; or Z^{2a} optionally forms a heterocycle with one or more R¹, R², Q¹, or A³;
each A³ is independently selected from PRT, H, —OH, —C(O)OH, cyano, alkyl, alkenyl, alkynyl, amino, amido, imido, imino, halogen, CF₃, —OCF₃, CH₂CF₃, cycloalkyl, nitro, aryl, aralkyl, alkoxy, aryloxy, heterocycle, —C(A²)₃, —C(A²)₂-C(O)A², —C(O)A², —C(O)OA², —O(A²), —N(A²)₂, —S(A²), —CH₂P(Y¹)(A²)(OA²), —CH₂P(Y¹)(A²)(N(A²)₂), —CH₂P(Y¹)(OA²)(OA²), —OCH₂P(Y¹)(OA²)(OA²), —OCH₂P(Y¹)(A²)(OA²), —OCH₂P(Y¹)(A²)(N(A²)₂), —C(O)OCH₂P(Y¹)(OA²)(OA²), —C(O)OCH₂P(Y¹)(A²)(OA²), —C(O)OCH₂P(Y¹)(A²)(N(A²)₂), —CH₂P(Y¹)(OA²)(N(A²)₂), —OCH₂P(Y¹)(OA²)(N(A²)₂), —C(O)OCH₂P(Y¹)(OA²)(N(A²)₂), —CH₂P(Y¹)(N(A²)₂)(N(A²)₂), —C(O)OCH₂P(Y¹)(N(A²)₂)(N(A²)₂), —OCH₂P(Y¹)(N(A²)₂)(N(A²)₂), —(CH₂)ₘ-heterocycle, —(CH₂)ₘC(O)Oalkyl, —O—(CH₂)ₘ—O—C(O)—Oalkyl, —O—(CH₂)ᵣ—O—C(O)—(CH₂)ₘ-alkyl, —(CH₂)ₘO—C(O)—O-alkyl, —(CH₂)ₘO—C(O)—O-cycloalkyl, —N(H)C(Me)C(O)O-alkyl, SRᵣ, S(O)Rᵣ, S(O)₂Rᵣ, Si(R³)₃, or alkoxy arylsulfonamide,
wherein each A³ may be optionally substituted with
1 to 4 —R¹, —P(Y¹)(OA²)(OA²), —P(Y¹)(OA²)(N(A²)₂), —P(Y¹)(A²)(OA²), —P(Y¹)(A²)(N(A²)₂), or P(Y¹)(N(A²)₂)(N(A²)₂), —C(═O)N(A2)₂), halogen, alkyl, alkenyl, alkynyl, aryl, carbocycle, heterocycle, aralkyl, aryl sulfonamide, aryl alkylsulfonamide, aryloxy sulfonamide, aryloxy alkylsulfonamide, aryloxy arylsulfonamide, alkyl sulfonamide, alkyloxy sulfonamide, alkyloxy alkylsulfonamide, arylthio, —(CH₂)ₘheterocycle, —(CH₂)ₘ—C(O)O-alkyl, —O(CH₂)ₘOC(O)Oalkyl, —O—(CH₂)ₘ—O—C(O)—(CH₂)ₘ-alkyl, —(CH₂)ₘ—O—C(O)—O-alkyl, —(CH₂)ₘ—O—C(O)—O-cycloalkyl, —N(H)C(CH₃)C(O)O-alkyl, or alkoxy arylsulfonamide, optionally substituted with R¹;
optionally each independent instance of A³ and Q¹ can be taken together with one or more A³ or Q¹ groups to form a ring;
A² is independently selected from PRT, H, alkyl, alkenyl, alkynyl, amino, amino acid, alkoxy, aryloxy, cyano, haloalkyl, cycloalkyl, aryl, heteroaryl, heterocycle, alkylsulfonamide, or arylsulfonamide, wherein each A² is optionally substituted with A³;
R^{f} is H, alkyl, alkenyl, alkynyl, aryl, heteroaryl, or cycloalkyl, which R^{f} is optionally substituted with one or more R_{g};
each R_{g} is independently alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
each Rₕ and Rⱼ is independently H, alkyl, or haloalkyl;
m is 0 to 6;
Z¹ is -L¹-A⁴-L²-A⁵;
L¹ is a bond, (C1-10)alkyl, O, S, —C(═O)—, —C(═O)O—, —OC(═O)—, —C(═O)NR³—, —NR³C(═O)—, —S(O)—, —S(O)₂—, —NR³S(O)₂—, —S(O)₂NR³—, or NR³;
A⁴ is a monocyclic heteroaryl containing 1, 2, or 3 N, which A⁴ is optionally substituted with one or more A³;
L² is (C1-10)alkyl, O, S, —C(═O)—, —C(═O)O—, —OC(═O)—, —C(═O)NR³—, —NR³C(═O)—, —S(O)—, —S(O)₂—, —NR³S(O)₂—, —S(O)₂NR³—, or NR³; and
A⁵ is aryl, alkyl, cycloalkyl, or heteroaryl, which A⁵ is optionally substituted with one or more A³.

2. The compound of claim 1 wherein R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

3. The compound of claim 1 wherein R^{f} is cyclopropyl.

4. The compound of claim 1 wherein R^{f} is 1-methylcyclopropyl.

5. The compound of claim 1 which is a compound of formula (II): 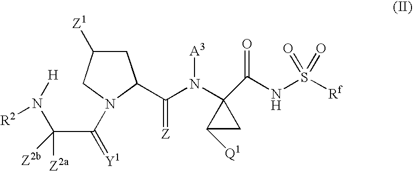
or a pharmaceutically acceptable salt, or prodrug thereof, wherein: Rⱼ is tert-butoxycarbonyl, cyclopentyloxycarbonyl, 2,2,2-trifluoro-1,1-dimethylethyl, 1-methylcyclopropyloxycarbonyl, 2-(N,N-dimethylamino)-1-1-dimethylethoxycarbonyl, 2-morpholino-1-1-dimethylethoxycarbonyl, tetrahydrofur-3-yloxycarbonyl, or

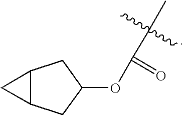

6. The compound of claim 5 wherein Z is O; Y¹ is O; and one of Z^{2a} and Z^{2b} is hydrogen.

7. The compound of claim 1 wherein Q¹ is vinyl, ethyl, cyanomethyl, propyl, 2-fluoroethyl, 2,2-difluoroethyl, or 2-cyanoethyl.

8. The compound of claim 1 wherein Q¹ and Z^{2a} taken together with the atoms to which they are attached form a 12-18 membered heterocycle, which heterocycle may optionally be substituted with one or more oxo (═O) or A³.

9. The compound of claim 1 which is a compound of formula (III): 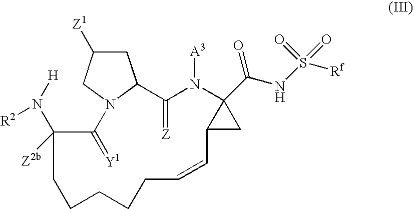
or a pharmaceutically acceptable salt, or prodrug thereof.

10. The compound of claim 1 which is a compound of formula (IV): 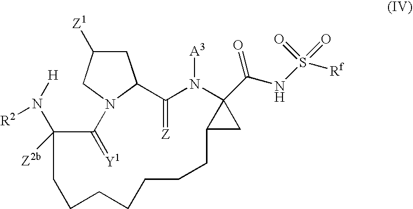
or a pharmaceutically acceptable salt, or prodrug thereof.

11. The compound of claim 1 wherein Z^{2a} is tert-butyl, 1-methylcyclohexyl, tetrahydropyran-4-yl, 1-methylcyclohexyl, 4,4-difluorocyclohexyl, 2,2,2-trifluoro-1-trifluoromethylethyl, or cyclopropyl.

12. The compound of claim 1 wherein X is O, S, or NR³.

13. The compound of claim 1 wherein X is O.

14. The compound of claim 1 wherein Y is a polycarbocycle.

15. The compound of claim 1 wherein Y is polyheterocycle.

16. The compound of claim 1 wherein Y is a fused carbocyclic ring system.

17. The compound of claim 1 wherein Y is a fused heterocyclic ring system.

18. The compound of claim 1 wherein Y is a fused carbocyclic ring system comprising one or more double bonds.

19. The compound of claim 1 wherein Y is a fused heterocyclic ring system comprising one or more double bonds.

20. The compound of claim 1 wherein Y is a bridged carbocyclic ring system.

21. The compound of claim 1 wherein Y is a bridged heterocyclic ring system.

22. The compound of claim 1 wherein Y is a bridged carbocyclic ring system comprising one or more double bonds.

23. The compound of claim 1 wherein Y is a bridged heterocyclic ring system comprising one or more double bonds.

24. The compound of claim 1 wherein Y comprises a bridged ring system selected from: 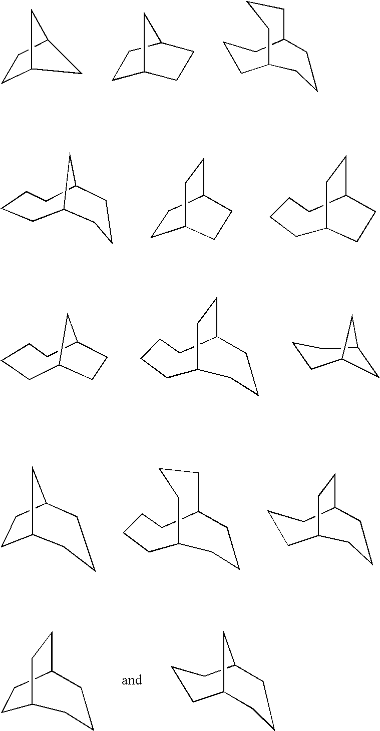
wherein one or more carbon atoms in the bridged ring system is optionally replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo; and wherein the ring system optionally comprises one or more double bonds.

25. The compound of claim 24 wherein the ring system comprises one or more double bonds.

26. The compound of claim 24 wherein one or more carbon atoms in the bridged ring system is replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo.

27. The compound of claim 1 wherein Y comprises a fused ring system selected from: 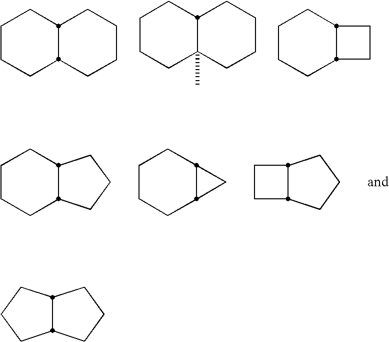
wherein one or more carbon atoms in the fused ring system is optionally replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo; and wherein the ring system optionally comprises one or more double bonds.

28. The compound of claim 27 wherein one or more carbon atoms in the bridged ring system is replaced with O, S, S(O), S(O)₂, N⁺(O⁻)Rₓ, or NRₓ; wherein each Rₓ is independently H, (C1-10)alkyl, (C2-10)alkenyl, (C2-10)alkynyl, (C1-10)alkanoyl, S(O)₂NRₙRₚ, S(O)₂Rₓ, or (C1-10)alkoxy, wherein each (C1-10)alkyl, (C2-10)alkenyl, (C2-10)alkynyl, (C1-10)alkanoyl, and (C1-10)alkoxy is optionally substituted with one or more halo.

29. The compound of claim 1 wherein Y is selected from:

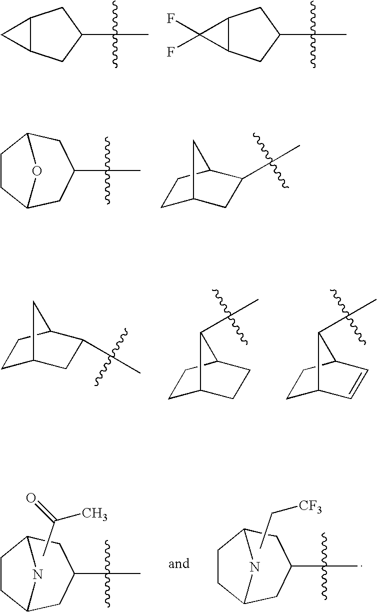

30. The compound of claim 1 wherein L¹ is O.

31. The compound of claim 1 wherein A⁴ is a pyrimidine or triazine ring that is optionally substituted with one or more A³.

32. The compound of claim 31 wherein each A³ is independently selected from halogen, aryl, heterocycle, —N(A²)₂, or SRᵣ wherein each A³ may be optionally substituted with 1 to 4-R¹.

33. The compound of claim 1 wherein A⁴ is a pyrimidinyl or triazinyl ring that is optionally substituted with one or more chloro, pyrrolidinyl, piperidinyl, morpholino, 2-hydroxyethylamino, dimethylamino, isopropylamino, piperazinyl, cyclopentylamino, imidazolyl, 1,2,4-triazolyl, phenyl, N-(trimethylsilylmethyl)amino, 1,3-thiazol-2-yl, methylthio, 4-fluorophenylamino, methoxy, or

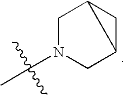

34. The compound of claim 1 wherein L² is a O, S, or NR³.

35. The compound of claim 1 wherein A⁵ is a phenyl, tetrazolyl, thiadiazolyl, cyclopentyl, or thiazolyl ring that is optionally substituted with one or more A³.

36. The compound of claim 35 wherein each A³ is independently fluoro, methyl, trifluoromethyl, or trifluoromethoxy.

37. The compound of claim 1 wherein -L²-A⁵ is selected from:

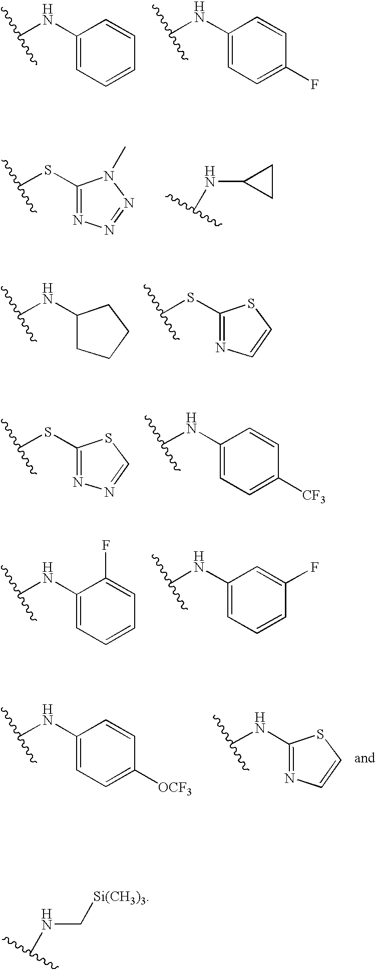

38. The compound of claim 1 wherein Z¹ is selected from:

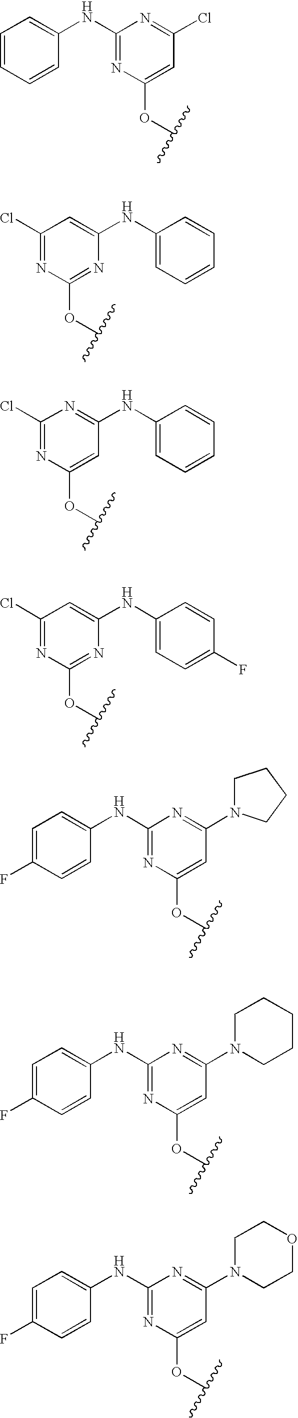

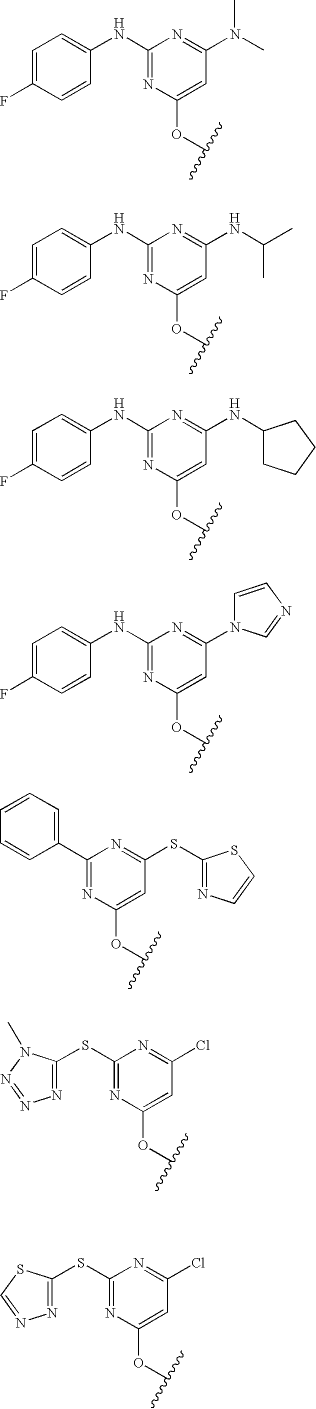

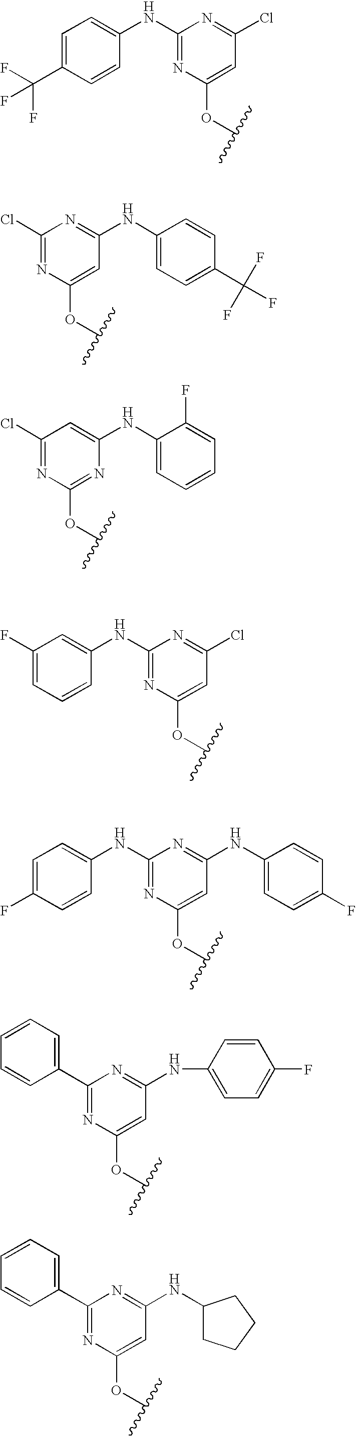

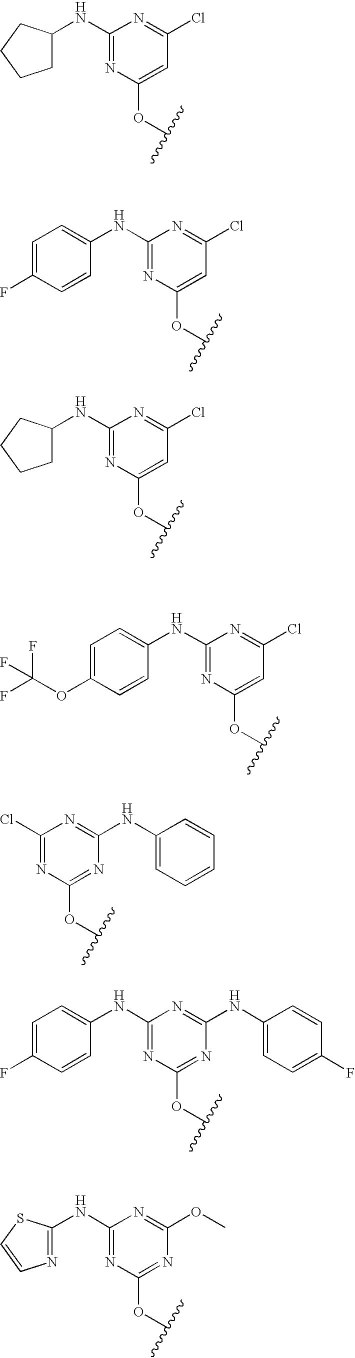

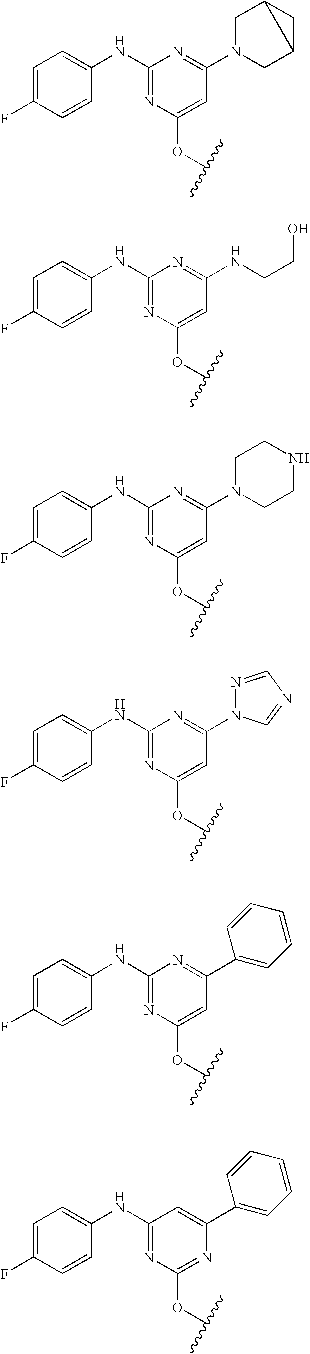

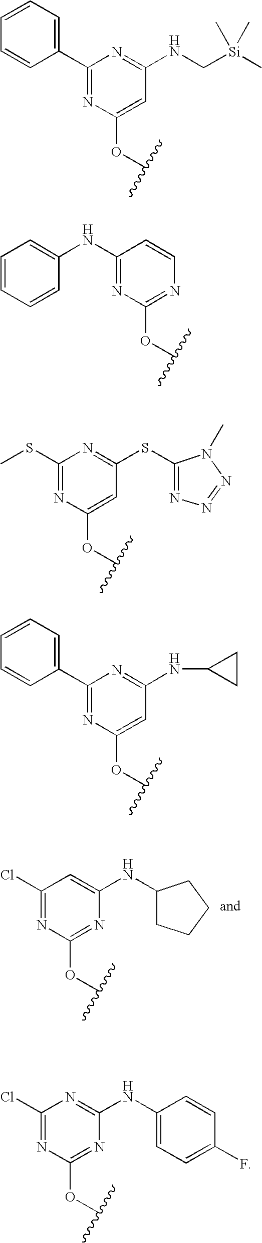

39. The compound of claim 1 wherein A⁵ is aryl, (C₂-C₁₀)alkyl, cycloalkyl, or heteroaryl, which A⁵ is optionally substituted with one or more A³.

40. The compound of claim 1 wherein A⁵ is aryl, cycloalkyl, or heteroaryl, which A⁵ is optionally substituted with one or more A³.

41. The compound of claim 1 wherein A⁵ is alkyl or cycloalkyl substituted with one or more A³.

42. The compound of claim 1 wherein A⁵ is alkyl or cycloalkyl substituted with Si(R³)₃.

43. The compound of claim 1 which is 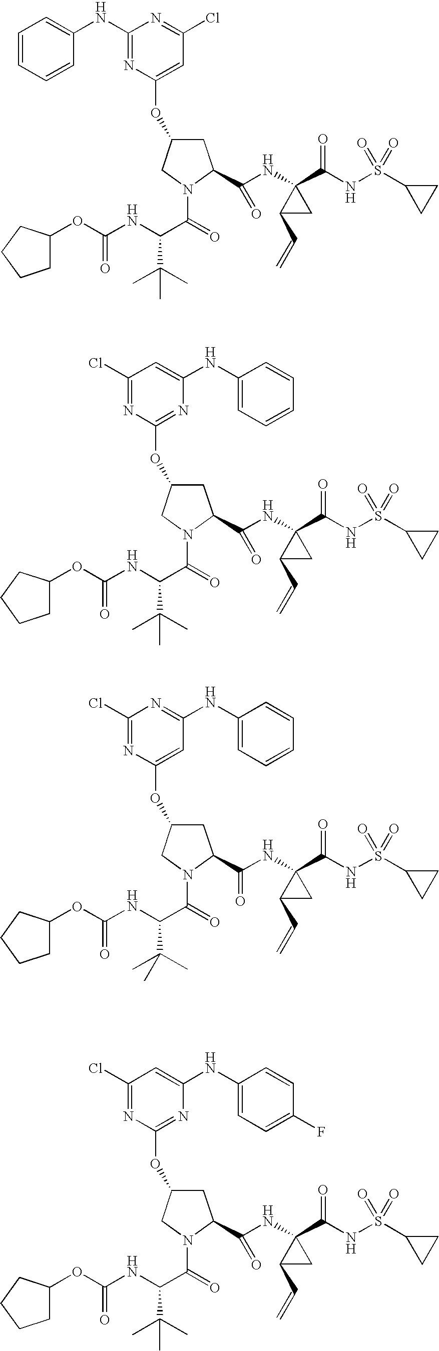 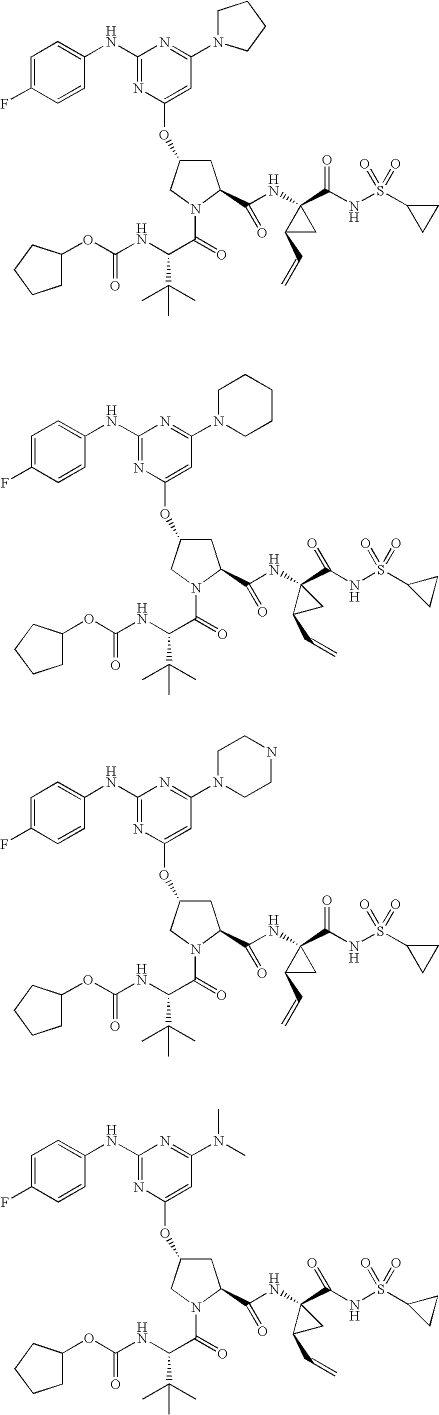 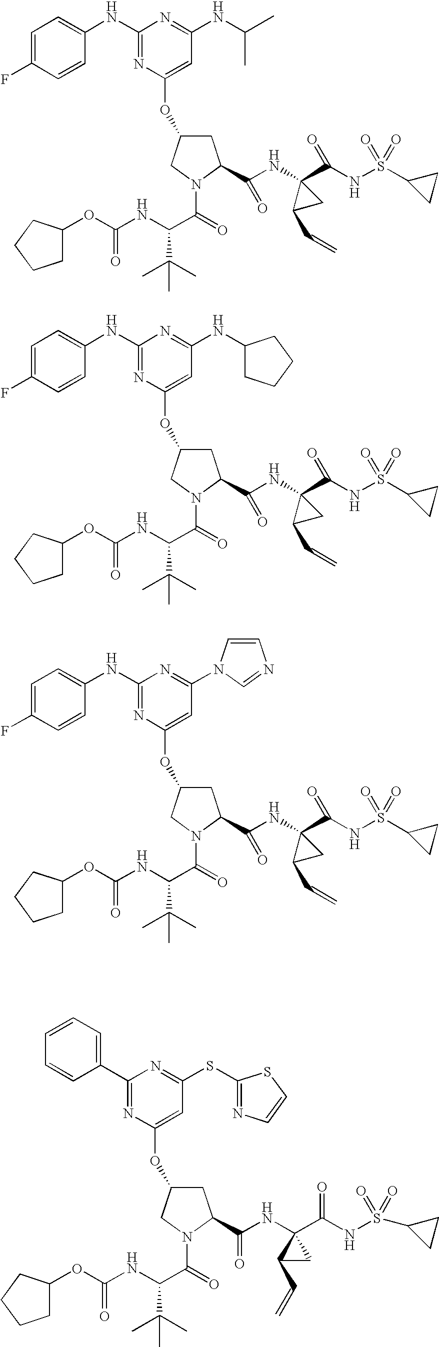 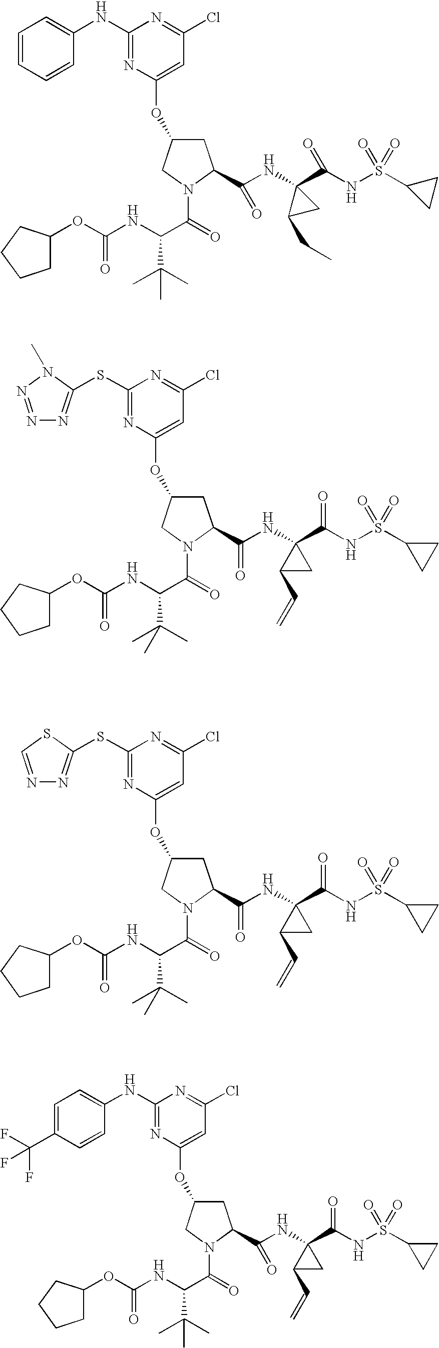 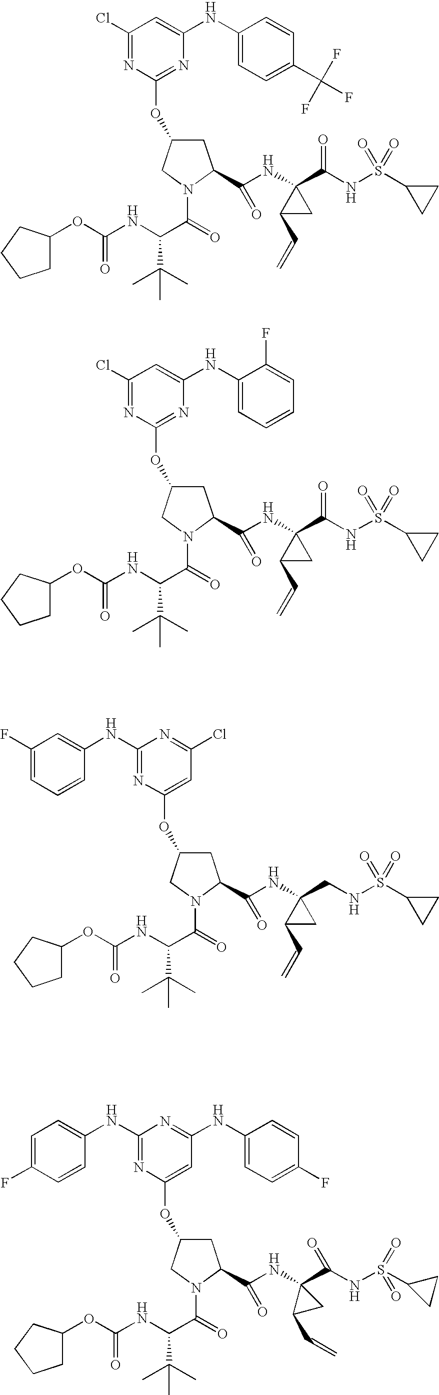 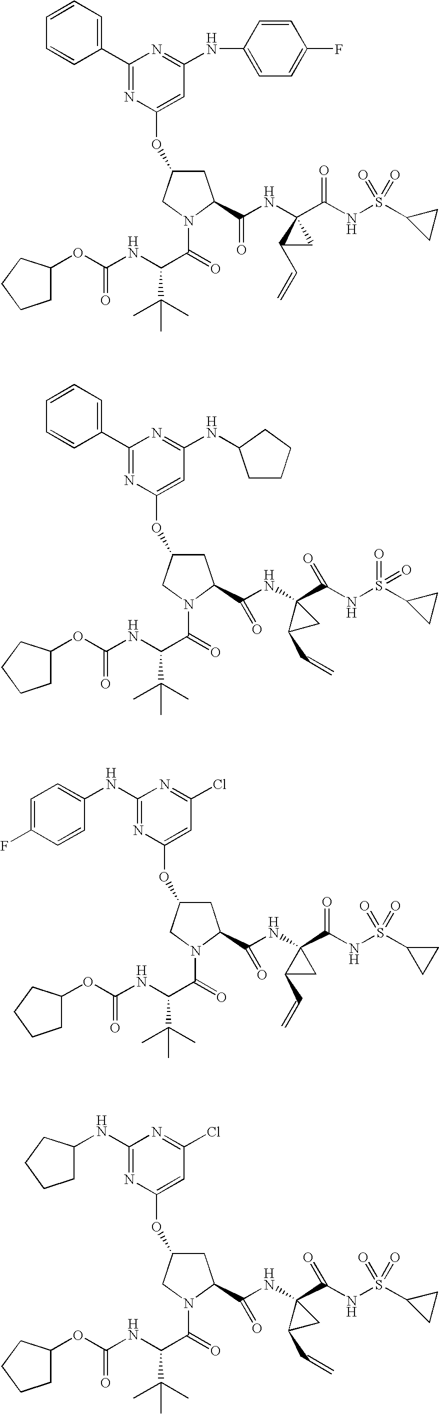 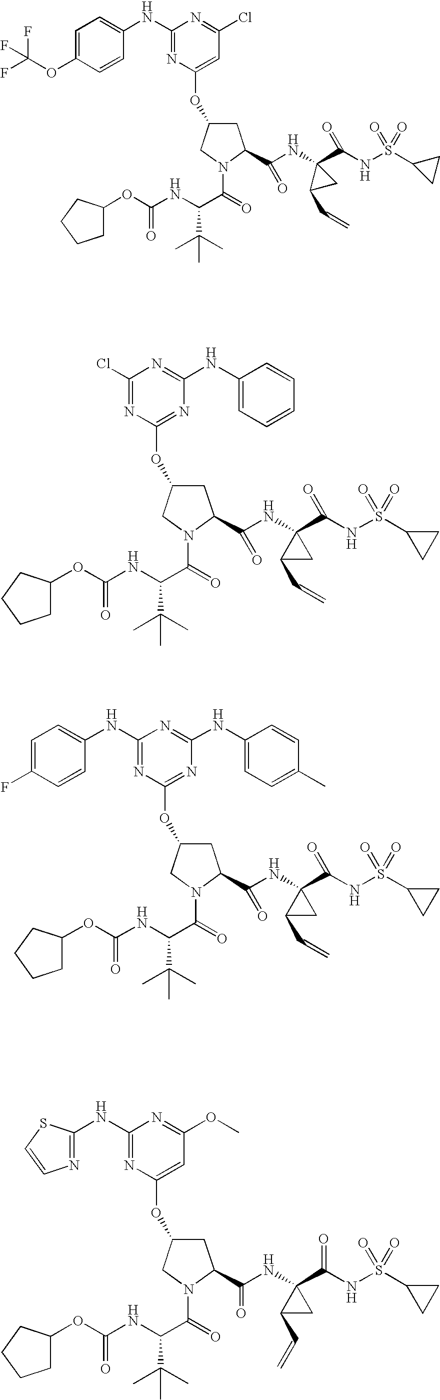 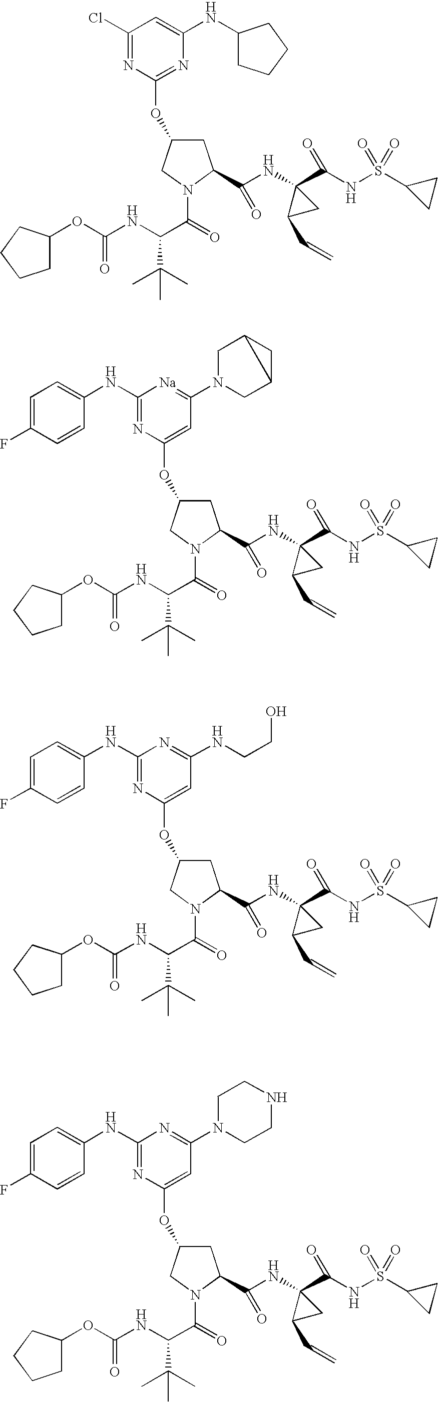 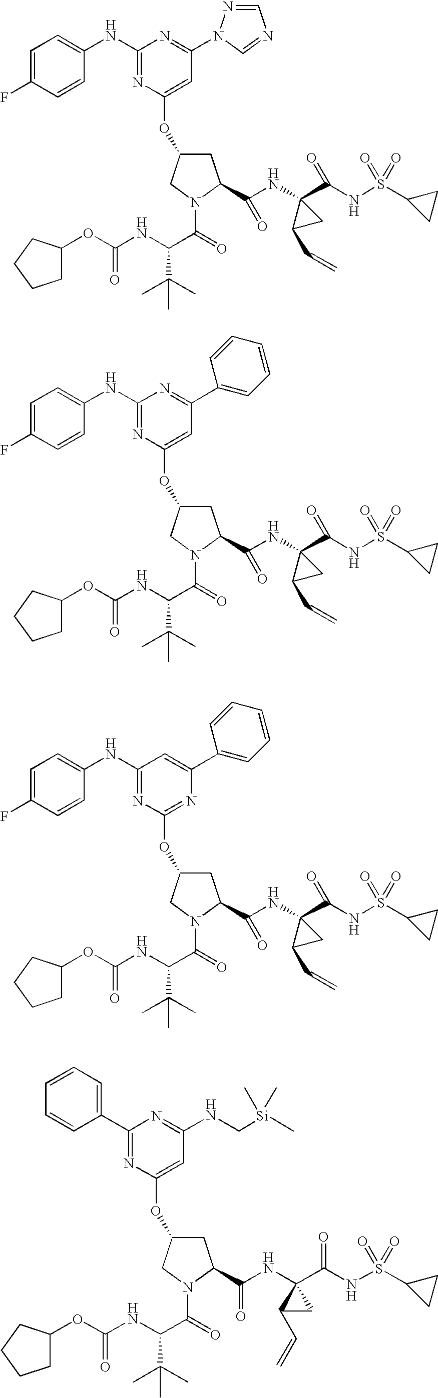 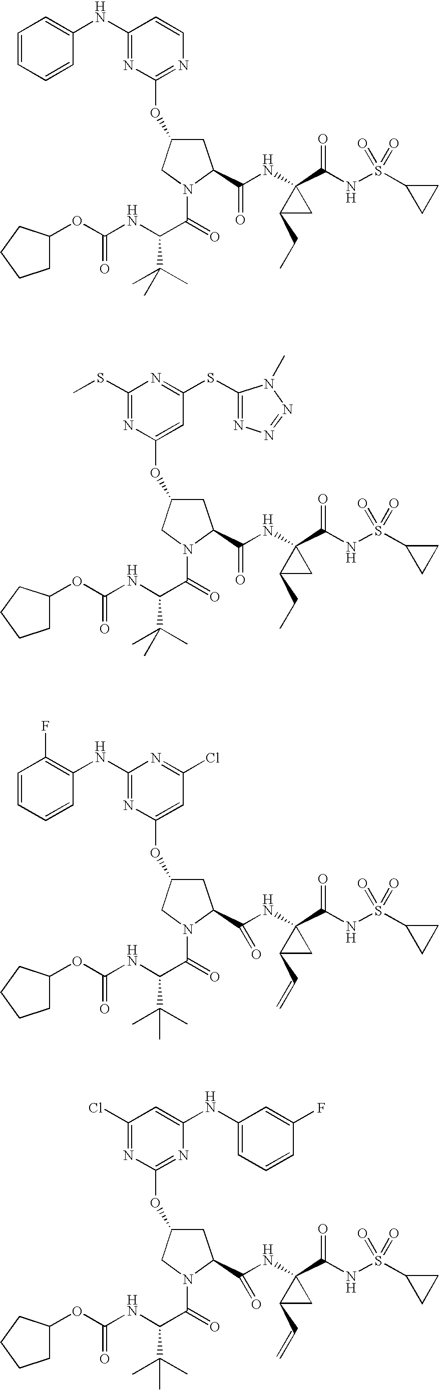 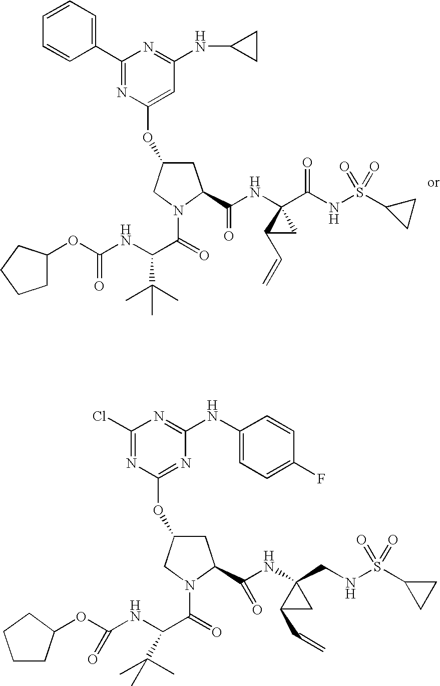
or a pharmaceutically acceptable salt, or prodrug thereof.

44. The compound of claim 1 which is 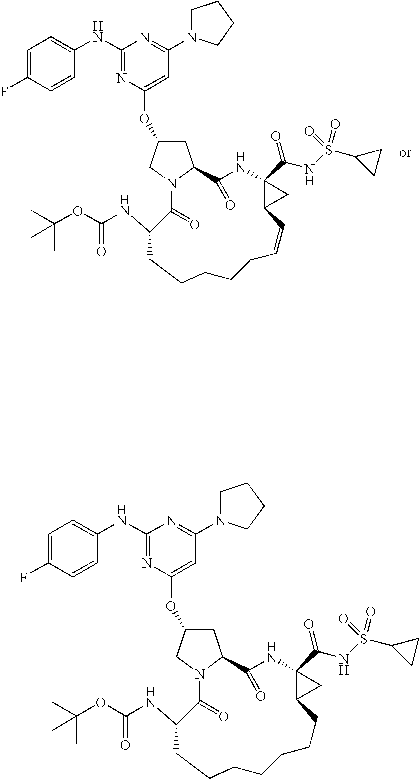
or a pharmaceutically acceptable salt, or prodrug thereof.

45. The compound of claim 1 which is a prodrug or a pharmaceutically acceptable salt thereof.

46. A pharmaceutical composition comprising the compound of formula I as described in claim 1, or a pharmaceutically acceptable salt thereof, and at least one pharmaceutically acceptable carrier.

47. The pharmaceutical composition according to claim 46 for use in treating disorders associated with HCV.

48. The pharmaceutical composition of claim 46, further comprising at least one additional therapeutic agent.

49. The pharmaceutical composition of claim 48, wherein said additional therapeutic agent is selected from the group consisting of interferons, ribavirin analogs, NS3 protease inhibitors, NS5b polymerase inhibitors, alpha-glucosidase 1 inhibitors, hepatoprotectants, non-nucleoside inhibitors of HCV, and other drugs for treating HCV.

50. The pharmaceutical composition according to claim 46, further comprising a nucleoside analogue.

51. The pharmaceutical composition according to claim 50, further comprising an interferon or pegylated interferon.

52. The pharmaceutical composition according to claim 51, wherein said nucleoside analogue is selected from ribavirin, viramidine, levovirin, a L-nucleoside, and isatoribine and said interferon is a-interferon or pegylated interferon.

53. A method of treating disorders associated with hepatitis C, said method comprising administering to an individual a pharmaceutical composition which comprises a therapeutically effective amount of the compound of formula I as described in claim 1, or a pharmaceutically acceptable salt thereof.
